(19) United States
(12) Patent Application Publication
(10) Pub. No.: US 2025/0270280 A1
(43) Pub. Date: Aug. 28, 2025

(54) MODIFIED BINDING PROTEINS AND THERAPEUTIC USES THEREOF

(71) Applicants: Monash University, Clayton, Victoria (AU); Queensland University of Technology, Brisbane, Queensland (AU)

(72) Inventors: Stephen Robert DALEY, Clayton, Victoria (AU); Stephanie GRAS, Clayton, Victoria (AU); Nicole LA GRUTA, Clayton, Victoria (AU); Pirooz ZAREIE, Clayton, Victoria (AU); Christopher SZETO, Clayton, Victoria (AU)

(73) Assignees: Monash University, Clayton, Victoria (AU); Queensland University of Technology, Brisbane, Queensland (AU)

(21) Appl. No.: 18/697,393

(22) PCT Filed: Oct. 12, 2022
(86) PCT No.: PCT/AU2022/051227

§ 371 Date: Mar. 29, 2024

(30) Foreign Application Priority Data
Oct. 12, 2021 (AU) 2021903279

(51) Int. Cl. C07K 14/725; A61K 38/00; C12N 5/0783; C12N 15/86

(52) U.S. Cl. CPC C07K 14/7051; C12N 5/0637; C12N 15/86; A61K 38/00; C07K 2317/565; C12N 2740/10043

(57) ABSTRACT

The present invention relates to modified T cell receptors and their uses in treating various diseases or conditions, particularly cancer and autoimmune diseases. A binding protein comprising a variable domain comprising a complementarity-determining region (CDR) capable of contacting a peptide bound to an HLA molecule, wherein the CDR 5 comprises a cysteine capable of forming a disulphide bond with a cysteine in the peptide bound to the HLA molecule, typically wherein the cysteine is introduced into the CDR by mutation or modification of an existing residue.

### FIELD OF THE INVENTION

The present invention relates to modified binding proteins, for example T cell receptors, and their uses in treating various diseases or conditions, particularly cancer and autoimmune diseases.

### RELATED APPLICATION

The present application is a 371 National Phase Application of PCT/AU2022/051227, filed Oct. 12, 2022, which application claims priority from Australian provisional application AU 2021903279, filed Oct. 12, 2021, the entire contents of which are hereby incorporated by reference.

### BACKGROUND OF THE INVENTION

T cell receptors (TCRs) mediate the recognition of specific antigens by T cells and, as such, are essential to the functioning of the cellular arm of the immune system. The native TCR is a heterodimeric cell surface protein of the immunoglobulin superfamily which is associated with invariant proteins of the CD3 complex involved in mediating signal transduction. TCRs exist in αβ and γδ forms, which are structurally similar but have quite distinct anatomical locations and functions. TCRs recognise antigens in the form of peptides presented by Major Histocompatibility Complex (MHC) proteins. The MHC class I and class II ligands are also immunoglobulin superfamily proteins but are specialised for antigen presentation, with a highly polymorphic peptide binding site which enables them to present a diverse array of peptide fragments at the surface of the antigen-presenting cell (APC).

Two further classes of proteins are known to be capable of functioning as TCR ligands. CD1 antigens are MHC class I-related molecules whose genes are located on a different chromosome from the classical MHC class I and class II. CD1 molecules are capable of presenting peptide and non-peptide (e.g. lipid, glycolipid) moieties to T cells in a manner analogous to conventional class I and class II MHC-peptide complexes. See, for example (Barclay et al, (1997) The Leucocyte Antigen Factsbook 2nd Edition, Academic Press) and (Bauer (1997) Eur J Immunol 27 (6) 1366-1373)). Bacterial superantigens are soluble toxins which are capable of binding both class II MHC molecules and a subset of TCRs (Fraser (1989) Nature 339 221-223). Many superantigens exhibit specificity for one or two Vbeta segments, whereas others exhibit more promiscuous binding. In any event, superantigens are capable of eliciting an enhanced immune response by virtue of their ability to stimulate subsets of T cells in a polyclonal fashion.

The extracellular portion of native heterodimeric αβ and γδ TCRs consist of two polypeptides each of which has a membrane-proximal constant domain, and a membrane-distal variable domain. Each of the constant and variable domains includes an intra-chain disulphide bond and the two chains of each heterodimer are linked by an inter-chain disulphide bond. The variable domains contain the highly polymorphic loops analogous to the complementarity determining regions (CDRs) of antibodies. CDR3 of αβ TCRs predominantly interact with the peptide presented by MHC, and CDRs 1 and 2 of αβ TCRs predominantly interact with the peptide and the MHC. The diversity of TCR variable domain sequences is generated via somatic rearrangement of linked variable (V), diversity (D), and joining (J) genes.

T cell activation depends on αβ T cell antigen receptor (TCR) co-recognition of peptide antigens presented by MHC molecules on the surface of APCs.

Recent advances in cellular immunotherapy have involved the adoptive transfer of T cells that efficiently recognize tumor antigens. These T cells are derived from tumor-infiltrating lymphocytes (TILs), and may also be peripheral blood T cells that can efficiently recognize tumor antigens after being genetically modified with antigen-specific TCRs (TCR-T). Adoptive T cell therapy includes chimeric antigen receptor (CAR) based CAR-T therapy as well as T cell receptor based TCR-T therapy. Unlike CAR T cells that recognize proteins expressed on the surface, TCRs can recognize tumor-specific proteins on the inside of cells.

A major challenge for TCR gene therapy is to achieve binding events between TCR and peptide-MHC (pMHC) that last long enough to elicit T cell stimulation. Further, there are limitations to the current TCR therapy as low-abundance peptides may fail to stimulate T cells.

There is a need for new and/or improved cell-based therapies, particularly T cell-based therapies, or TCR-based therapies, for the treatment of various conditions, including cancer and autoimmune diseases.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

### SUMMARY OF THE INVENTION

The present inventors have identified that it is possible to form a covalent linkage between a TCR complementarity-determining region (CDR) and a peptide bound to an MHC protein, also called a human leukocyte antigen (HLA) molecule, typically a disulphide bond formed between a cysteine in the TCR CDR and a cysteine in the peptide bound to the MHC molecule.

In one aspect, the present invention provides a binding protein comprising a variable domain comprising a complementarity-determining region (CDR) capable of contacting a peptide bound to an HLA molecule, wherein the CDR comprises a cysteine capable of forming a disulphide bond with a cysteine in the peptide bound to the HLA molecule.

In any aspect of the invention, the binding protein may be an antigen binding protein, for example an antibody or antigen-binding fragment thereof, or a T cell receptor or fragment thereof. In one embodiment, the T cell receptor is a soluble T cell receptor. In one embodiment the antigen binding protein or T cell receptor may be part of a chimeric or fusion protein, for example, the antigen binding protein may be part of a chimeric antigen receptor (CAR). In one embodiment, the chimeric or fusion protein comprises a soluble T cell receptor of the invention as described herein.

In any aspect of the invention, where the binding protein is an antibody, the variable domain may be a heavy chain variable domain or a light chain variable domain. Preferably the CDR is a CDR3. Therefore, the CDR3 containing the cysteine may be in the heavy chain variable domain or a light chain variable domain.

In any aspect of the invention, where the binding protein is a T cell receptor, the variable domain may be an α chain variable domain (Vα) or a β chain variable domain (Vβ). Therefore, the CDR3 containing the cysteine may be in the α chain or β chain. Preferably, the binding protein comprises both an α chain variable domain and a β chain variable domain, wherein the CDR that comprises a cysteine capable of forming a disulphide bond with a cysteine in the peptide bound to the HLA molecule is present in the α chain variable domain or a β chain variable domain, but not both. For example, the CDR3 may comprise, consist essentially of or consist of an amino acid sequence as shown in Table 3 or 4 with a cysteine residue present in the indicated position.

In one embodiment, the TCR has an α chain variable domain comprising a CDR3 shown in Table 3 or 4 and a β chain variable domain comprising a CDR3 shown in Table 3 or 4, wherein the residue indicated in the Vα CDR3 or Vβ CDR3 is replaced with a cysteine.

In any aspect of the invention, the HLA may be any HLA molecule or any HLA-like molecule that can present a peptide antigen on the cell surface. In any embodiment, the HLA may be a HLA class I or a HLA class II. Further, the HLA may be an HLA-like molecule such as HLA-E. HLAs corresponding to MHC class I include HLA-A, HLA-B, and HLA-C. HLAs corresponding to MHC class II include HLA-DP, HLA-DQ, and HLA-DR.

In any aspect of the invention, the binding protein may be recombinant, synthetic, purified or substantially purified.

Typically, the cysteine is introduced into the CDR by mutation or modification of an existing residue.

In any aspect of the invention, the CDR is preferably a CDR3.

In any aspect of the invention, the cysteine is present in the CDR at a position that allows formation of a disulphide bond with a cysteine present in a peptide bound to a HLA molecule, wherein the cysteine in the peptide is at one of positions P1, P2, P3, P4, P5, P6, P7, P8 or P9, wherein for MHC class I, the peptide position (P2) is the amino acid that occupies, or is closest to, the B pocket of the MHC class I molecule, and for MHC class II, the peptide position 1 is the amino acid that occupies, or is closest to, the P1 pocket of MHC class II molecule.

In any aspect of the invention, the cysteine is present in the CDR at a position that allows formation of a disulphide bond with a cysteine present in a peptide bound to a HLA class I molecule, wherein the cysteine in the peptide is at one of positions P4, P5, P6, P7, P8 or P9, wherein for MHC class I, the peptide position (P2) is the amino acid that occupies, or is closest to, the B pocket of the MHC class I molecule, and for MHC class II, the peptide position 1 is the amino acid that occupies, or is closest to, the P1 pocket of MHC class II molecule.

In any aspect of the invention, the cysteine is present in the CDR at a position that allows formation of a disulphide bond with a cysteine present in a peptide bound to a HLA class II molecule, wherein the cysteine in the peptide is at one of positions P1, P2, P4, P5, P6, P7 or P8, wherein for MHC class I, the peptide position (P2) is the amino acid that occupies, or is closest to, the B pocket of the MHC class I molecule, and for MHC class II, the peptide position 1 is the amino acid that occupies, or is closest to, the P1 pocket of MHC class II molecule.

In one embodiment, where the CDR is present in a TCR α-chain variable domain, the cysteine is present in the CDR at a position that allows formation of a disulphide bond with a cysteine present in a peptide bound to a HLA molecule, preferably the cysteine in the peptide is at one of positions P4, P5 or P6. Preferably, the HLA is a HLA class I. In another embodiment, where the CDR is present in a TCR β-chain variable domain, the cysteine is present in the CDR at a position that allows formation of a disulphide bond with a cysteine present in a peptide bound to an HLA molecule, preferably the cysteine in the peptide is at one of positions P4, P5, P6, P7, P8 or P9. Preferably, the HLA is a HLA class I.

In one embodiment, where the CDR is present in a TCR α-chain variable domain, the cysteine is present in the CDR at a position that allows formation of a disulphide bond with a cysteine present in a peptide bound to a HLA molecule, wherein the cysteine in the peptide is at one of positions P1, P2 or P5. Preferably, the HLA is a HLA class II. In another embodiment, where the CDR is present in a TCR β-chain variable domain, the cysteine is present in the CDR at a position that allows formation of a disulphide bond with a cysteine present in a peptide bound to a HLA molecule, wherein the cysteine in the peptide is at one of positions P4, P5, P6, P7 or P8. Preferably, the HLA is a HLA class II.

In any aspect of the present invention, where the CDR is present in a TCR α-chain variable domain, the cysteine is present in the CDR at a position that allows formation of a disulphide bond with the cysteine present in a peptide as shown in Table 2 or 3 bound to an HLA class I molecule.

In any aspect of the present invention, where the CDR is present in a TCR β-chain variable domain, the cysteine is present in the CDR at a position that allows formation of a disulphide bond with the cysteine present in a peptide as shown in Table 2 or 3 bound to an HLA class I molecule.

In any aspect of the present invention, where the CDR is present in a TCR α-chain variable domain, the cysteine is present in the CDR at a position that allows formation of a disulphide bond with the cysteine present in a peptide as shown in Table 1 or 4 bound to an HLA class II molecule.

In any aspect of the present invention, where the CDR is present in a TCR β-chain variable domain, the cysteine is present in the CDR at a position that allows formation of a disulphide bond with the cysteine present in a peptide as shown in Table 1 or 4 bound to an HLA class II molecule.

In any aspect of the present invention, the peptide may be bound to an HLA class I molecule selected from HLA-A, HLA-B, HLA-C or HLA-E.

In any aspect of the present invention, the peptide may be bound to an HLA class I molecule select from HLA-A*02:01, HLA-B*07:02, HLA-B*44:05, HLA-A*02:01, HLA-B*35:01, HLA-A*01:01, HLA-B*35:08, HLA-B*37:01, HLA-B*08:01, HLA-A*11:01, HLA-B*27:05, HLA-A*24:02, HLA-B*51:01, and HLA-E*01:03.

In any aspect of the present invention, the peptide may be bound to an HLA class II molecule selected from HLA-DR, HLA-DP, or HLA-DQ.

In any aspect of the present invention, the peptide may be bound to an HLA class II molecule select from HLA-DQA1*0508_HLA-DQB1*0201, HLA-DQA1*0501_HLA-DQB1*0201, HLA-DQA1*0301_HLA-DQB1*0302, HLA-DRA*0101_HLA-DBR1*0101, HLA-DRA*0101_HLA-DRB3*0301, HLA-DRA*0101_HLA-DBR5*0101, HLA-DRA*0101_HLA-DBR1*0401, HLA-DPA1*0103_HLA-DPB1*2602, HLA-DQA1*0501_HLA-DQB1*0302, HLA-DRA*0101_HLA-DRB1*1101, HLA-DRA*0101_HLA-DRB1*1502, HLA-DRA*0101_HLA-DRB1*0101, HLA-DQA1*0301_HLA-DQB1*0305, HLA-DQA1*0201_HLA-DQB1*0201.

In any aspect of the present invention, the cysteine capable of forming a disulphide bond with a cysteine in a peptide bound to a HLA molecule is present in the CDR at a position 3, 4, 5, 6, 7, 8, 9 or 10, wherein the numbering is relative to the amino acid at the N-terminus of the CDR (i.e. the amino acid at the N-terminus of the CDR is position 1).

In any aspect of the present invention, where the CDR is present in a TCR α-chain variable domain, the cysteine is present in the CDR at a position 3, 4, 5, 6, 7, 8, 9 or 10, wherein the numbering is relative to the amino acid at the N-terminus of the CDR (i.e. the amino acid at the N-terminus of the CDR is position 1). Preferably, the cysteine allows formation of a disulphide bond with a cysteine present in a peptide bound to a HLA class I molecule, more preferably with a cysteine present in position P4, P5 or P6.

In any aspect of the present invention, where the CDR is present in a TCR β-chain variable domain, the cysteine is present in the CDR at a position 3, 4, 5, 6, 7, 8, 9 or 10 wherein the numbering is relative to the amino acid at the N-terminus of the CDR (i.e. the amino acid at the N-terminus of the CDR is position 1). Preferably, the cysteine allows formation of a disulphide bond with a cysteine present in a peptide bound to a HLA class II molecule, more preferably with a cysteine present in position P4, P5, P6, P7, P8 or P9.

In any aspect of the present invention, where the CDR is present in a TCR α-chain variable domain, the cysteine is present in the CDR at a position 5, 6, 7, 8 or 11 wherein the numbering is relative to the amino acid at the N-terminus of the CDR (i.e. the amino acid at the N-terminus of the CDR is position 1). Preferably, the cysteine allows formation of a disulphide bond with a cysteine present in a peptide bound to a HLA class II molecule, more preferably with a cysteine present in position P1, P2 or P5.

In any aspect of the present invention, where the CDR is present in a TCR β-chain variable domain, the cysteine is present in the CDR at a position 5, 6, 7, 8 or 9 wherein the numbering is relative to the amino acid at the N-terminus of the CDR (i.e. the amino acid at the N-terminus of the CDR is position 1). Preferably, the cysteine allows formation of a disulphide bond with a cysteine present in a peptide bound to a HLA class II molecule, more preferably with a cysteine present in position P4, P5, P6, P7 or P8. In any aspect of the present invention, the peptide bound to a HLA molecule may contain a cysteine that naturally occurs in, or has been introduced in, any one of the positions referred to herein.

In another aspect, the present invention provides a chimeric or fusion protein comprising a binding protein of any aspect of the invention.

In another aspect, the present invention provides a nucleic acid comprising, consisting essentially of or consisting of a nucleotide sequence encoding a binding protein, or chimeric or fusion protein of any aspect of the invention.

In another aspect, the present invention provides a vector comprising a nucleic acid of any aspect of the invention, or a nucleotide sequence encoding a binding protein, or chimeric or fusion protein of any aspect of the invention. Typically, the vector allows expression of the nucleotide sequence in a cell resulting in the presentation of the binding protein on the surface of the cell. The vector may be a retroviral vector, preferably a lentiviral vector. Typically, the vector allows expression of the nucleotide sequence in a T cell, preferably a T helper cell, for example a CD4+ T cell. A CD4+ T cell may be a T regulatory cell (Treg), preferably a CD4+CD25high T cell. Alternatively, the T cell may be a CD8+ T cell.

In one embodiment, the vector comprises a nucleic acid of the invention operably linked to a promoter.

In embodiments of the invention directed to single polypeptide chain binding protein, the expression construct may comprise a promoter linked to a nucleic acid encoding that polypeptide chain.

In embodiments of the invention directed to multiple polypeptide chains that form a binding protein, a vector comprises a nucleic acid encoding a polypeptide comprising, e.g., a Vα operably linked to a promoter and a nucleic acid encoding a polypeptide comprising, e.g., a Vβ operably linked to a promoter.

In another example, the expression construct is a bicistronic expression construct, e.g., comprising the following operably linked components in 5′ to 3′ order:
(i) a promoter;
(ii) a nucleic acid encoding a first polypeptide;
(iii) an internal ribosome entry site, preferably, 2A peptide cleavage motif derived from a Picornavirus; and
(iv) a nucleic acid encoding a second polypeptide;
wherein the first polypeptide comprises a Vα and the second polypeptide comprises a Vβ, or vice versa. Preferably, the vector allows translation of the nucleotide sequence encoding Vβ before translation of the nucleotide sequence encoding Vα.

In another embodiment, the present invention also contemplates separate vectors one of which encodes a first polypeptide comprising a Vα and another of which encodes a second polypeptide comprising a Vβ. For example, the present invention also provides a composition comprising:
(i) a first expression construct comprising a nucleic acid encoding a polypeptide comprising a Vα operably linked to a promoter; and
(ii) a second expression construct comprising a nucleic acid encoding a polypeptide comprising a Vβ operably linked to a promoter.

In another aspect, the invention provides a cell comprising a vector or nucleic acid described herein. Preferably, the cell is isolated, substantially purified or recombinant. In one example, the cell comprises the vector of the invention or:
(i) a first expression construct comprising a nucleic acid encoding a polypeptide comprising a Vα operably linked to a promoter; and
(ii) a second expression construct comprising a nucleic acid encoding a polypeptide comprising a Vβ operably linked to a promoter;
wherein the first and second polypeptides associate to form a binding protein of the present invention. Preferably, the cell is a T cell, more preferably a T helper cell, for example a CD4+ T cell. A CD4+ T cell may be a T regulatory cell (Treg), preferably a CD4+CD25high T cell.

In another aspect, the present invention provides a cell expressing on its surface a binding protein of the invention. Preferably, the cell is a T cell, more preferably a CD4+ T cell. A CD4+ T cell may be a T regulatory cell (Treg), preferably a CD4+CD25high T cell.

In another aspect, the present invention provides a method of preparing a population of T regulatory cells for use in the treatment of an autoimmune disease, the method comprising:
providing a population of T regulatory cells,
introducing a nucleic acid or vector of the invention into the population of T regulatory cells,
providing conditions to allow the expression of the binding protein, chimeric or fusion protein on the surface of the T regulatory cells,
thereby preparing a population of T regulatory cells for use in the treatment of an autoimmune disease. The autoimmune disease may be any one described herein.

In another aspect, the present invention provides a method of preparing a population of T regulatory cells for use in the treatment or prevention of transplant rejection, the method comprising:
providing a population of T regulatory cells,
introducing a nucleic acid or vector of the invention into the population of T regulatory cells,
providing conditions to allow the expression of the binding protein, chimeric or fusion protein on the surface of the T regulatory cells,
thereby preparing a population of T regulatory cells for use in the treatment or prevention of transplant rejection.

In another aspect, the present invention provides a method of preparing a population of cytotoxic T cells for use in the treatment of cancer or an infectious disease, the method comprising:
providing a population of cytotoxic T cells,
introducing a nucleic acid or vector of the invention into the population of cytotoxic T cells,
providing conditions to allow the expression of the binding protein, chimeric or fusion protein on the surface of the cytotoxic T cells,
thereby preparing a population of cytotoxic T cells for use in the treatment of cancer or an infectious disease. The cancer or infectious disease may be any described herein.

In another aspect, the present invention relates to a method for preparing an ex vivo population of T cells exhibiting at least one property of a regulatory T cell, the method comprising:
providing a population of T cells exhibiting at least one property of a regulatory T cell;
introducing a nucleic acid or vector of the invention into the population of T cells, wherein the nucleic acid or vector encodes a binding protein, chimeric or fusion protein of the invention; and
providing conditions to allow the expression of the binding protein, chimeric or fusion protein on the surface of the T cells;
thereby preparing an ex vivo population of T cells exhibiting at least one property of a regulatory T cell. Preferably, T cells exhibiting at least one property of a regulatory T cell are derived from a biological sample from a subject having an autoimmune disease.

The T cells exhibiting at least one property of a regulatory T cell used in a method or use of the invention may be selected from a subject diagnosed with an autoimmune disease or from a healthy subject(s). The T cells may be isolated from a histocompatible donor.

In alternative embodiments, the present invention provides a method of preparing an ex vivo population of T cells exhibiting at least one property of a regulatory T cell, the method comprising:
providing a population of T cells exhibiting at least one property of a conventional T cell, optionally wherein the population of T cells is a mixed population of T cells;
introducing a nucleic acid or vector of the invention into the population of T cells, wherein the nucleic acid or vector encodes a binding protein, chimeric or fusion protein of the invention;
providing conditions to allow the expression of the binding protein, chimeric or fusion protein on the surface of the T cells; and
providing conditions to allow conversion of the population of T cells into T regulatory cells;
thereby preparing an ex vivo population of T cells exhibiting at least one property of a regulatory T cell. Preferably, the T cells exhibiting at least one property of a conventional T cell or mixed population of T cells are derived from a biological sample from a subject having an autoimmune disease. Alternatively, the T cell may be derived from a histocompatible donor.

In another aspect, the present invention relates to a method for preparing an ex vivo population of T cells exhibiting at least one property of a cytotoxic T cell, the method comprising:
providing a population of T cells exhibiting at least one property of a cytotoxic T cell;
introducing a nucleic acid or vector of the invention into the population of T cells, wherein the nucleic acid or vector encodes a binding protein, chimeric or fusion protein of the invention; and
providing conditions to allow the expression of the binding protein, chimeric or fusion protein on the surface of the T cells;
thereby preparing an ex vivo population of T cells exhibiting at least one property of a cytotoxic T cell. Preferably, T cells exhibiting at least one property of a cytotoxic T cell are derived from a biological sample from a subject having cancer or an infectious disease.

The T cells exhibiting at least one property of a cytotoxic T cell used in a method or use of the invention may be selected from a subject diagnosed with a cancer or an infectious disease or from a healthy subject(s). The T cells may be isolated from a histocompatible donor.

In alternative embodiments, the present invention provides a method of preparing an ex vivo population of T cells exhibiting at least one property of a cytotoxic T cell, the method comprising:
providing a population of T cells exhibiting at least one property of a conventional T cell, optionally wherein the population of T cells is a mixed population of T cells;
introducing a nucleic acid or vector of the invention into the population of T cells, wherein the nucleic acid or vector encodes a binding protein, chimeric or fusion protein of the invention;
providing conditions to allow the expression of the binding protein, chimeric or fusion protein on the surface of the T cells; and
providing conditions to allow conversion of the population of T cells into cytotoxic T cells;
thereby preparing an ex vivo population of T cells exhibiting at least one property of a cytotoxic T cell. Preferably, the T cells exhibiting at least one property of a conventional T cell or mixed population of T cells are derived from a biological sample from a subject having cancer or an infectious disease. Alternatively, the T cell may be derived from a histocompatible donor.

In another aspect, the present invention also relates to a composition of T regulatory cells wherein greater than 20% of the cells express a binding protein, chimeric or fusion protein of the invention. Preferably, the composition includes greater than 30%, 40%, 50%, 60%, 70%, 80%, 85%, 90%, 91%, 92%, 93%, 94%, 95%, 96%, 97%, 98 or 99% of cells that express a binding protein, chimeric or fusion protein of the invention.

In another aspect, the present invention also relates to a composition of cytotoxic T cells wherein greater than 20% of the cells express a binding protein, chimeric or fusion protein of the invention. Preferably, the composition includes greater than 30%, 40%, 50%, 60%, 70%, 80%, 85%, 90%, 91%, 92%, 93%, 94%, 95%, 96%, 97%, 98 or 99% of cells that express a binding protein, chimeric or fusion protein of the invention.

In any aspect or embodiment, the conditions for allowing conversion of a conventional T cell or mixed population of T cells, into a T regulatory cell may comprise contacting the conventional T cells or mixed population of T cells with one or more agents, or increasing the expression of one or more factors suitable for conversion of conventional T cells into regulatory T cells. The one or more agents or factors may comprise: TGF-β, Foxp3 or an agent for increasing expression thereof.

In another aspect, the present invention provides a method of treating or preventing an autoimmune disease in a subject, the method comprising administering to the subject a binding protein, chimeric or fusion protein, nucleic acid, cell, or composition of the invention, thereby treating or preventing the autoimmune disease in the subject.

In another aspect, the present invention provides a method of treating or preventing transplant rejection in a subject, the method comprising administering to the subject a binding protein, chimeric or fusion protein, nucleic acid, cell, or composition of the invention, thereby treating or preventing transplant rejection in the subject.

In another aspect, the present invention provides a method of treating or preventing cancer or an infectious disease in a subject, the method comprising administering to the subject a binding protein, chimeric or fusion protein, nucleic acid, cell, or composition of the invention, thereby treating or preventing the cancer or infectious disease in the subject.

In another aspect, the present invention provides use of a binding protein, chimeric or fusion protein, nucleic acid, cell or composition of the invention in the manufacture of a medicament for treating or preventing an autoimmune disease in a subject.

In another aspect, the present invention provides use of a binding protein, chimeric or fusion protein, nucleic acid, cell or composition of the invention in the manufacture of a medicament for treating or preventing transplant rejection in a subject.

In another aspect, the present invention provides use of a binding protein, chimeric or fusion protein, nucleic acid, cell or composition of the invention in the manufacture of a medicament for treating or preventing cancer or an infectious disease in a subject.

In another aspect, the present invention provides a binding protein, chimeric or fusion protein, peptide, cell, nucleic acid or composition of the invention for use in treating or preventing cancer or an infectious disease in a subject.

In another aspect, the present invention provides a binding protein, chimeric or fusion protein, peptide, cell, nucleic acid or composition of the invention for use in treating or preventing an autoimmune disease in a subject.

In another aspect, the present invention provides a binding protein, chimeric or fusion protein, peptide, cell, nucleic acid or composition of the invention for use in treating or preventing transplant rejection in a subject.

In another aspect, the present invention provides a method for identifying a residue location in a CDR3 of a binding protein that, when a cysteine is present at that residue location, could form a disulphide bond with a cysteine present in a peptide/MHC, the method comprising
analysing a TCR-peptide/MHC interface, or antibody or antigen-binding fragment thereof/MHC interface, for one or more residues in close contact between the CDR3 and a cysteine residue in the peptide;
mutating the residue in the CDR3 that is identified as being in close contact to a cysteine residue in the peptide;
determining that a disulphide bond is likely to form if the interatomic distance between the sulphur atom in the cysteine introduced in the CDR3 and the sulphur atom in the cysteine in the peptide is between 0.01 and 3 Å (angstroms) and the dihedral angle between planes through the two sets of S-S-C_{β} atoms (with the S-S atoms in common) is less than 30° from either −87° or +97°, thereby identifying the residue location in a CDR3 of a binding protein that, when a cysteine is present at that residue location, could form a disulphide bond with a cysteine present in a peptide/MHC without structural rearrangement of the CDR3 or the peptide.

In this aspect, the binding protein could be any binding protein described herein.

In this aspect, the TCR-peptide/MHC interface, or antibody or antigen-binding fragment thereof/MHC interface, may be determined by any known means, for example x-ray crystallography or NMR, or may be determined from a model of the interaction in silico.

In this aspect, mutating the residue in the CDR3 that is identified as being in close contact to a cysteine may be performed in silico.

In this aspect, the method further comprises confirming the formation of a disulphide bond between the CDR3 and peptide, further comprising the steps of
providing a nucleic acid encoding the CDR3, preferably encoding a binding protein comprising the CDR3;
introducing a cysteine at the residue location in the CDR3 of the binding protein that, when a cysteine is present at that residue location, could form a disulphide bond with a cysteine present in a peptide/MHC;
producing the binding protein comprising the CDR3 with the introduced cysteine;
providing the binding protein and peptide/MHC in conditions to allow disulphide bond formation;
determining the formation of a disulphide bond.

In this aspect, producing the binding protein comprising the CDR3 with the introduced cysteine may be by any method known in the art, including those methods described herein.

In another aspect, the present invention provides a method of producing a binding protein, or chimeric or fusion protein, of the invention as described herein, the method comprising culturing a cell comprising a nucleic acid or vector of the invention as described herein under conditions to allow expression of the binding protein, or chimeric or fusion protein, of the invention as described herein.

In another aspect, the present invention provides a method of producing a binding protein, or chimeric or fusion protein, of the invention as described herein, the method comprising the steps of
providing a nucleic acid encoding a CDR3, preferably encoding a binding protein, chimeric or fusion protein, comprising a CDR3;
introducing a cysteine at the residue location in the CDR3 of the binding protein, chimeric or fusion protein, that, when a cysteine is present at that residue location, could form a disulphide bond with a cysteine present in a peptide/MHC;
producing the binding protein, chimeric or fusion protein, comprising the CDR3 with the introduced cysteine.

In this aspect, producing the binding protein, chimeric or fusion protein, comprising the CDR3 with the introduced cysteine may be by any method known in the art, including those methods described herein. Further, the method may further comprise a step of purifying or isolating the binding protein.

In another aspect, the present invention provides a method of identifying a mutant TCR with a decreased rate of dissociation with its target peptide bound to an HLA (pHLA) compared to the unmutated TCR, the method comprising:
creating a plurality of TCRs having mutations to introduce a cysteine residue in an α chain CDR3 sequence and/or a β chain CDR3 sequence,
determining the interactions of members of said plurality of mutant TCRs with the target pHLA, and
selecting one or more members having a decreased rate of dissociation with the target pHLA compared to the unmutated TCR.

In another aspect, the present invention provides a method of identifying a mutant TCR with a decreased rate of dissociation with its target peptide bound to an HLA (pHLA) compared to the unmutated TCR, the method comprising:
creating a plurality of TCRs having mutations to introduce a cysteine residue in an α chain CDR3 sequence and/or a β chain CDR3 sequence,
determining the interactions of members of said plurality of mutant TCRs with the target pHLA, and
selecting one or more members having a decreased rate of dissociation with the target pHLA compared to the unmutated TCR, wherein the decreased rate of dissociation is due to formation of a disulphide bond,
optionally confirming the formation of a disulphide bond between the introduced cysteine residue and a cysteine in the target pHLA.

In one embodiment, the decreased rate of dissociation is at least 5, 10 or 20-fold.

In one embodiment, the dissociation rate between a TCR (mutated or unmutated) and pHLA is determined by cell-based tetramer dissociation assays and/or surface plasmon resonance assays.

In one embodiment, the peptide is a low abundant peptide. In another embodiment, the TCR prior to introduction of a cysteine residue has an affinity of greater than or equal to 1 mM, 100 μM, 10 μM, 1 μM, or 100 nM for the given pHLA. Introducing a cysteine may be desirable across this entire range to enhance low-affinity interactions or enhance high-affinity interactions.

As used herein, except where the context requires otherwise, the term “comprise” and variations of the term, such as “comprising”, “comprises” and “comprised”, are not intended to exclude further additives, components, integers or steps.

Further aspects of the present invention and further embodiments of the aspects described in the preceding paragraphs will become apparent from the following description, given by way of example and with reference to the accompanying drawings.

### BRIEF DESCRIPTION OF THE DRAWINGS

FIG. 1. TCR and peptide antigen sequences. (A) Details of the 6218, 6218αC and 6218βC TCRs. (B) Names and sequences of peptide antigens used in the drawings. These peptide antigens bind to the mouse MHC class I protein, H2-D^{b}.

FIG. 2. Confirmation of disulphide bond formation using X-ray crystallography. (A) Superposition of three TCR-pMHC structures with the 6218 TCR-PA/H2-D^{b} complex in pink, the 6218 TCR-PA4C/H2-D^{b} in gold, and the 6218αC TCR-PA4C/H2-D^{b} in purple. (B) Zoom in view on the TCR-peptide interface with the addition of the PA4C/H2-D^{b} structure with the peptide in pale blue. (C) Top view of the H2-D^{b} antigen-binding cleft in white cartoon with the mass centre (sphere) of each variable domain of the TCR from the three complexes aligned on panel (A) (same colour coding). (D) Structure of the 6218 TCR in complex with PA/H2-D^{b}, with the TCR and peptide in pink and H2-D^{b} in white. (E) Structure of the 6218 TCR (gold) in complex with PA4C peptide (gold) presented by H2-D^{b} (white). (F) Structure of the 6218αC TCR (blue) in complex with PA4C/H2-D^{b} (peptide in blue, MHC in white) showing a disulphide bond formed at the interface. (G) Superposition of 6218 TCR-PA/H2-D^{b} (pink) and 6218αC TCR-PA4C/H2-D^{b} (blue) complexes in the same orientation as in (D) and (F).

FIG. 3. Disulphide bond formation increases T-cell sensitivity to peptide antigen. Supernatant IL-2 concentration after a 16 h co-culture of the 5KC T-cell line expressing CD8αβ, and either the 6218 TCR or 6218αC TCR, with the DC2.4 cell line with graded concentrations of PA4C peptide. Control cultures included 5KC cells with 50 μM PA4C peptide (T only), 5KC and DC2.4 cells without peptide (T+DC) and 5KC cells with plate-bound anti-CD3 (α-CD3). Symbols show the mean and error bars the range of 2 wells per condition. Data are representative of 3 separate experiments.

FIG. 4. An assay for disulphide bonding between an immobilised TCR and soluble peptide/MHC. (A) Surface plasmon resonance (SPR) sensorgrams of immobilised 6218 (black) or 6218αC (red) TCRs exposed to PA4C/H2-D^{b} sequentially injected at increasing concentrations. (B) Abrogation of persistent binding by reducing agent. PA4C/H2-D^{b} monomers were incubated in reducing agent (DTT) overnight before SPR analysis as in (A). (C) Progressive disulphide bond formation over time. The immobilised 6218αC TCR was first exposed to 1-minute injections of negative (irrelevant pMHC) and positive (PA/H2-D^{b}) control pMHC monomers (not shown), before injection of PA4C/H2-D^{b} for 1, 5 or 20 minutes followed by injection of buffer. (D) Short half-life of short-lived 6218αC TCR-PA4C/H2-D^{b} complexes. SPR data for a 12-s interval spanning the transition from pMHC monomer injection to buffer injection. Some data points between −0.5 s and 0 s are outside the limits of the y-axis. Sensorgrams are aligned so that the onset of measurable dissociation occurs at time=0.1 s. Error bars show the range of 2 experiments for 6218 TCR-PA/H2-D^{b}, 3 experiments for 6218 TCR-PA4C/H2-Db (2 without DTT and 1 with DTT) and 1 experiment for 6218αC TCR-PA4C/H2-D^{b} (with DTT).

FIG. 5. An exposed cysteine in CDR3 promotes above-threshold TCR signaling and T-cell tolerance induction in the thymus. (A) Altered development of thymocytes with Cys-containing CDR3. Bone marrow (BM) cells pooled from Rag1^{-/-} mice (7 female and 6 male; aged 37-134 d) were transduced with retroviruses encoding GFP and the 6218, 6218αC or 6218βC TCR and then were mixed 1:1 with T cell-depleted wild-type BM cells from a male B6 mouse before injection into irradiated Rag1^{-/-} mice (3 female and 1 male for 6218; 4 female for 6218αC and 5 male for 6218RC). The resulting TCR-retrogenic mice were analysed 5 weeks after BM transfer at 90-171 days of age. Plots show fluorescence-activated cell sorting (FACS) phenotypes of live thymocytes analysing GFP/CCR7 (top row) and GFP/TCRβ (bottom row); with a gate for GFP+ TCRβ+ thymocytes that were analysed for CD4/CD8β(row 3) and PD-1/NK1.1 (row 4). Phenotypes of lymphocytes from (B) spleen were analysed for GFP/TCRβ (top row) with a gate for the GFP⁺ TCRβ⁺ subset, which was analysed for CD8α/CD8β phenotype (bottom row). Phenotypes of CD45+ cells from (C) small intestine were analysed for GFP/TCRγδ (top row) with a gate for the GFP⁺ TCRγδ⁻ subset, which was analysed for CD8α/CD8β phenotype (bottom row). In (A), (B) and (C), graphs show the percentage of gated events, or the absolute number (#) of gated events per mouse determined by multiplying the percentage of gated events by the total number of cells per organ. Each symbol in a graph represents one mouse; circle symbols for 6218, square symbols for 6218αC, and triangle symbols for 6218βC. Statistical analyses used 1-way ANOVA with Tukey's multiple comparisons test.

FIG. 6. An exposed cysteine in CDR3 affects the abundance and distribution of developing T cells in the thymus. Immunofluorescence histology of thymus from TCR-retrogenic mice expressing the 6218 or 6218αC TCR. Thymus sections were stained for GFP, with medullary areas identified by staining for cytokeratin-14 (K14). Dashed lines demarcate cortex (C) from medulla (M). Scale bars: 200 μm. TCR-retrogenic mice were analysed 5 weeks after bone marrow transfer at 99-138 days of age (n=4 for 6128; n=4 for 6218αC). Graph summaries (below) show the GFP⁺ cell density (number of GFP+ cells per section, top, or per mm², bottom) in cortical and medullary areas. Each symbol in a graph represents one mouse. Statistical analyses used unpaired two-tailed student's t tests.

FIG. 7. Cysteine residues do not affect the probability of TCR binding to closely related pMHC tetramers in a cell-based tetramer staining assay. (A) Substitutions at P7 of the PA4C peptide decrease TCR binding by pMHC tetramers. Plots show FACS phenotypes of TCR transfectants expressing the 6218 or 6218αC TCR stained with anti-TCRβ and H2-D^{b} tetramers in complex with the indicated peptide (top). Numbers on plots show the tetramer mean fluorescence intensity (MFI) divided by TCRβ MFI, normalized to the 6218 TCR-PA/H2-D^{b} sample. (B) For the indicated TCR-peptide/H2-D^{b} combinations (right), symbols show the mean tetramer binding, calculated as in (A), as a function of the K_{eq} determined by surface plasmon resonance (SPR), with dotted lines connecting measurements from the same pMHC. Error bars show the range of 3-6 samples from 2-3 experiments (y-axis) or the standard error of the mean of 2 experiments with a total of 4 samples per combination (x-axis).

FIG. 8. Substitutions at P7 of the peptide affect the probability of TCR binding to pMHC monomers regardless of the presence of cysteine residues. SPR sensorgrams for immobilised 6218 TCR (black) or 6218αC TCR (red) exposed to progressively increasing concentrations of the soluble pMHC monomer annotated on each graph. The sensorgrams on the left panels show the PA peptide and its variants without P4-Cys, while the sensorgrams on the right are for the PA4C peptide and its variants. The bottom panels are tables with the K_{D} values derived from the above sensorgams. The pink arrows show the level of pMHC retention after injection, indicative of disulphide bond formation.

FIG. 9. Disulphide bond formation prevents dissociation of TCR from peptide/MHC. (A) Progressive disulphide bond formation over time. SPR sensorgrams show binding to the 6218αC TCR by H2-D^{b} monomers (100 μM) in complex with PA4C (red), PA4C7K (blue), or PA4C7A (grey) injected for 20 min, or PA4C7L (black) injected for 50 min, followed by injection of buffer. (B) Disulphide bond formation inhibits tetramer dissociation. TCR transfectants expressing 6218 or 6218αC, which had been stained with pMHC tetramers as in FIG. 7, were washed and resuspended in buffer containing 25 μg/mL anti-H2-D^{b}/K^{b} to prevent tetramer rebinding for 10 min, 30 min or 60 min before FACS analysis. Graph shows the tetramer⁺ cell frequency as a percentage of the corresponding sample without anti-H2-D^{b}/K^{b} at time=0 min. Symbols show the mean and error bars the range of 4 samples per condition, compiled from 2 experiments.

FIG. 10. Disulphide bond formation results in increased T-cell sensitivity to peptide antigens, and decreased discrimination of peptide antigens, compared to noncovalent antigen recognition. (A) Supernatant IL-2 concentration after coculture of 5KC T cells expressing CD8αβ, and either the 6218 TCR or 6218αC TCR, with DC2.4 cells with graded concentrations of the indicated peptide (shown in upper left of each graph). Control cultures included 5KC cells with 50 μM peptide (T only), 5KC and DC2.4 cells without peptide (T+DC), and 5KC cells with plate-bound anti-CD3 (α-CD3). Symbols show the mean, and error bars the range, of 2 wells per condition from one experiment, representative of at least 2 experiments. HillSlope (h) values were determined using nonlinear regression. For curves that did not reach a plateau, the reported EC₅₀ values provide a minimum estimate of the EC₅₀. Data are presented as in FIG. 3 and are representative of 2 separate experiments. (B) For each TCR/peptide combination, graphs show relative tetramer binding (x-axes) plotted against EC₅₀ (left) or h (right) values, which were determined from a total of at least 4 dilution series per peptide analyzed in at least 2 experiments. Error bars indicate the 95% confidence intervals of EC₅₀ and h; dotted lines connect measurements from the same pMHC.

FIG. 11. Distribution of engineerable disulphide bonds in TCR-peptide/MHCI complexes. The panel summarises the analyses of TCR-peptide/MHCI complexes presented in Table 3 indicating the total number of distinct high-confidence engineerable TCR-peptide bonds involving the TCRα chain (above the x-axis) or the TCRβ chain (below the x-axis) with each graph representing a given position in the peptide (denoted above the graph).

FIG. 12. Distribution of engineerable disulphide bonds in TCR-peptide/MHCII complexes. The panel summarises the analyses of TCR-peptide/MHCII complexes presented in Table 4 indicating the total number of distinct high-confidence engineerable TCR-peptide bonds involving the TCRα chain (above the x-axis) or the TCRβ chain (below the x-axis) with each graph representing a given position in the peptide (denoted above the graph).

FIG. 13. Role of Zap70 and MHC in cysteine-linked T cell fate skewing. (A) Attenuation of TCR signalling disrupts the effect of Cys-containing CDR3 on T cell fate. Bone marrow (BM) cells pooled from Zap70^{mrd/mrt} mice (11 female and 9 male; aged 33-60 days) were transduced with retroviruses encoding GFP and the 6218, 6218αC or 6218βC TCR and then injected into irradiated male Zap70^{mrd/mrt} mice. Splenocytes from TCR-retrogenic mice were analysed for GFP/TCRβ phenotype with a gate for GFP⁺ TCRβ⁺ cells. Graph shows the absolute number of GFP⁺ TCRβ⁺ splenocytes with each symbol representing one mouse: 6218 (circle symbols), 6218αC (square symbols), 6218βC (triangle symbols). (B) Differential Cys-containing CDR3 expression in polyclonal T cell subsets requires strong TCR signaling in response to pMHC ligands. Sorted pre-selection thymocytes, small intestinal CD8αα IEL, splenic CD4⁺ T-conv and splenic CD8₊ T-conv populations were analysed by TCR sequencing. Graphs show the percentage of unique TCRα (circles) or TCRβ (crosses) sequences with Cys within 2 positions of the middle CDR3 amino acid (cysteine index) with each symbol on a graph representing a sample from one mouse: left panel, wild type (n=11; 6 females and 5 males, aged 45-133 days); middle panel, Zap70^{mrd/mrt} mice (n=3; aged 37-74 days); right panel, B2m^{-/-}H2-A^{-/-} mice (n=1 female and 4 males aged 98-140 days). p values in the left panel were determined using 2-way ANOVA with Sidak's multiple comparisons test.

FIG. 14: Context-dependent effects of Cys of TCR-pMHC binding. TCR transfectants expressing mouse CD3, GFP and the 6218, 6218αC or 6218βC TCR were incubated with anti-TCRβ and tetramers of H2-D^{b} presenting the indicated PA or PA4C peptide (left). FACS plots show tetramer staining versus TCRβ expression on live GFP⁺ TCRβ⁺ cells. Data are representative of 6 experiments for cells expressing the 6218 TCR or the 6218αC TCR, and 1 experiment for cells expressing the 6218βC TCR.

FIG. 15: Generation of a CD4⁺ T cell hybridoma that binds to the α3/DR15 antigen, as demonstrated by staining with α3/DR15-APC tetramers. FACS plots show tetramer staining versus CD4⁺ expression on hybridoma cells.

FIG. 16: T cell activation assays using LS1 hybridoma to identify reactivity to variants of the α3 peptide with cysteine substitutions at TCR-exposed positions.
(A) Histograms show fluorescein signal in LS1 hybridoma cells that had been incubated for 16 hours with CellTrace Violet (CTV)-labelled splenocytes from a naïve DR15-transgenic. Fcgr2b^{-/-}, mouse in the absence or presence of the peptide (50 μg/mL) named above each histogram. (B) FACS dot plots show fluorescein (x-axis) versus side scatter (y-axis) in LS1 hybridoma cells that had been incubated for 16 hours with CTV-labelled bone marrow-derived dendritic cells from a naïve DR15-transgenic. Fcgr2b^{-/-} mouse in the absence or presence of the peptide (50 μg/mL) named above each histogram.

FIG. 17: Details of variable (TRAV/TRBV) and junctional (TRAJ/TRBJ) gene segments and CDR3 amino acid sequences of TCRα and TCRβ chains expressed by the LS1 T cell hybridoma.

### DETAILED DESCRIPTION OF THE EMBODIMENTS

It will be understood that the invention disclosed and defined in this specification extends to all alternative combinations of two or more of the individual features mentioned or evident from the text or drawings. All of these different combinations constitute various alternative aspects of the invention.

Further aspects of the present invention and further embodiments of the aspects described in the preceding paragraphs will become apparent from the following description, given by way of example and with reference to the accompanying drawings.

Reference will now be made in detail to certain embodiments of the invention.

While the invention will be described in conjunction with the embodiments, it will be understood that the intention is not to limit the invention to those embodiments. On the contrary, the invention is intended to cover all alternatives, modifications, and equivalents, which may be included within the scope of the present invention as defined by the claims.

T cell activation depends on T cell antigen receptor (TCR) co-recognition of peptide antigens presented by Major Histocompatibility Complex (MHC) molecules on the surface of antigen-presenting cells (APC). The inventor(s) have found that TCR-peptide disulphide bonds stabilise the interaction between a TCR and its peptide/MHC ligand. The inventors have shown this with both cell-bound TCR and soluble TCR. Considering two interactions with high probabilities of TCR binding to pMHC but distinguished by the capacity to form a disulphide bond, the interaction that permits disulphide bond formation can facilitate T-cell activation at lower concentrations of peptide than the interaction that does not permit disulphide bond formation. Furthermore, considering two interactions with low probabilities of TCR binding to pMHC but distinguished by the capacity to form a disulphide bond, the interaction that permits disulphide bond formation can facilitate T-cell activation whereas the interaction that does not permit a disulphide bond cannot facilitate T-cell activation.

TCR gene therapy provides patients with T cells that are genetically engineered to express desired TCRs. Use of genetically engineered TCRs that form disulphide bonds with naturally expressed peptides thereby enhances TCR gene therapy of conventional T cells (TCR-T cell therapy) for cancer (1) and TCR gene therapy of T-regulatory cells (TCR Treg therapy) for autoimmune disease (2). This is because the lifetime of interactions between the TCR and peptide/MHC will no longer be a limiting factor in TCR gene therapy. Furthermore, a new field of lower-abundance peptides will become accessible targets for TCR gene therapy.

A key advantage of a TCR-peptide disulphide bond is the long half-life of the TCR-peptide/MHC interaction. This overcomes a major challenge for TCR gene therapy, namely, to achieve sufficient persistence of the TCR/peptide-MHC recognition unit to elicit T cell activation. An important result is that disulphide-permissive T cells can be activated by disulphide-permissive peptides with low expression levels, opening a new field of peptides to T cell recognition.

The cysteine-engineered TCRs may enable T-cell recognition of antigens that are previously not able to be targeted by TCR-T cell therapy, e.g. antigens that have low expression or low presentation by MHC to T cells, peptides presented by non-classical MHC (e.g. HLA-E, HLA-F).

TCR gene therapy provides patients with autologous T cells that are genetically engineered to express desired αβ TCRs. TCR gene therapy may be used to treat cancer. TCR gene therapy of T-regulatory cells has potential as a treatment for autoimmune disease. It is envisaged that TCR gene therapy using disulphide-permissive TCRs will enable T cell stimulation by low-abundance peptides that would otherwise fail to stimulate T cells.

### General

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or groups of compositions of matter. Thus, as used herein, the singular forms “a”, “an” and “the” include plural aspects, and vice versa, unless the context clearly dictates otherwise. For example, reference to “a” includes a single as well as two or more; reference to “an” includes a single as well as two or more; reference to “the” includes a single as well as two or more and so forth.

Those skilled in the art will appreciate that the present invention is susceptible to variations and modifications other than those specifically described. It is to be understood that the invention includes all such variations and modifications. The invention also includes all of the steps, features, compositions and compounds referred to or indicated in this specification, individually or collectively, and any and all combinations or any two or more of said steps or features.

One skilled in the art will recognize many methods and materials similar or equivalent to those described herein, which could be used in the practice of the present invention. The present invention is in no way limited to the methods and materials described.

All of the patents and publications referred to herein are incorporated by reference in their entirety.

The present invention is not to be limited in scope by the specific examples described herein, which are intended for the purpose of exemplification only. Functionally equivalent products, compositions and methods are clearly within the scope of the present invention.

Any example or embodiment of the present invention herein shall be taken to apply mutatis mutandis to any other example or embodiment of the invention unless specifically stated otherwise.

Unless specifically defined otherwise, all technical and scientific terms used herein shall be taken to have the same meaning as commonly understood by one of ordinary skill in the art (for example, in cell culture, molecular genetics, immunology, immunohistochemistry, protein chemistry, and biochemistry).

Unless otherwise indicated, the recombinant protein, cell culture, and immunological techniques utilized in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et al. Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

The description and definitions of variable regions and parts thereof, T cell receptors and fragments thereof herein may be further clarified by the discussion in Kabat Sequences of Proteins of Immunological Interest, National Institutes of Health, Bethesda, Md., 1987 and 1991, Bork et al., J Mol. Biol. 242, 309-320, 1994, Chothia and Lesk J. Mol Biol. 196:901-917, 1987, Chothia et al. Nature 342, 877-883, 1989 and/or or Al-Lazikani et al., J Mol Biol 273, 927-948, 1997.

The term “and/or”, e.g., “X and/or Y” shall be understood to mean either “X and Y” or “X or Y” and shall be taken to provide explicit support for both meanings or for either meaning.

As used herein the term “derived from” shall be taken to indicate that a specified integer may be obtained from a particular source albeit not necessarily directly from that source.

Reference herein to a range of, e.g., residues, will be understood to be inclusive. For example, reference to “a region comprising amino acids 1 to 15” will be understood in an inclusive manner, i.e., the region comprises a sequence of amino acids as numbered 1, 2, 3, 4, 5, 6, 7, 8, 9, 10, 11, 12, 13, 14 and 15 in a specified sequence.

The term “consisting essentially of” limits the scope of a claim to the specified materials or steps, or to those that do not materially affect the basic characteristics of a claimed invention. For example, a protein domain, region, or module (e.g., a binding domain, hinge region, linker module) or a protein (which may have one or more domains, regions, or modules) “consists essentially of” a particular amino acid sequence when the amino acid sequence of a domain, region, module, or protein includes extensions, deletions, mutations, or a combination thereof (e.g., amino acids at the amino- or carboxy-terminus or between domains) that, in combination, contribute to at most 20% (e.g., at most 15%, 10%, 8%, 6%, 5%, 4%, 3%, 2% or 1%) of the length of a domain, region, module, or protein and do not substantially affect (i.e., do not reduce the activity by more than 50%, such as no more than 40%, 30%, 25%, 20%, 15%, 10%, 5%, or 1%) the activity of the domain(s), region(s), module(s), or protein (e.g., the target binding affinity of a binding protein).

As used herein, “nucleic acid” or “nucleic acid molecule” refers to any of deoxyribonucleic acid (DNA), ribonucleic acid (RNA), oligonucleotides, fragments generated, for example, by the polymerase chain reaction (PCR) or by in vitro translation, and fragments generated by any of ligation, scission, endonuclease action, or exonuclease action. In certain embodiments, the nucleic acids of the present disclosure are produced by PCR. Nucleic acids may be composed of monomers that are naturally occurring nucleotides (such as deoxyribonucleotides and ribonucleotides), analogs of naturally occurring nucleotides (e.g., α-enantiomeric forms of naturally-occurring nucleotides), or a combination of both. Modified nucleotides can have modifications in or replacement of sugar moieties, or pyrimidine or purine base moieties. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, phosphoramidate, and the like. Nucleic acid molecules can be either single stranded or double stranded.

The term “isolated” means that the material is removed from its original environment (e.g., the natural environment if it is naturally occurring). For example, a naturally occurring nucleic acid or polypeptide present in a living animal is not isolated, but the same nucleic acid or polypeptide, separated from some or all of the co-existing materials in the natural system, is isolated. Such nucleic acid could be part of a vector and/or such nucleic acid or polypeptide could be part of a composition (e.g., a cell lysate), and still be isolated in that such vector or composition is not part of the natural environment for the nucleic acid or polypeptide. The term “gene” means the segment of DNA involved in producing a polypeptide chain; it includes regions preceding and following the coding region “leader and trailer” as well as intervening sequences (introns) between individual coding segments (exons).

As used herein, the term “recombinant” refers to a cell, microorganism, nucleic acid molecule, or vector that has been genetically engineered by human intervention—that is, modified by introduction of an exogenous or heterologous nucleic acid molecule, or refers to a cell or microorganism that has been altered such that expression of an endogenous nucleic acid molecule or gene is controlled, deregulated or constitutive. Human generated genetic alterations may include, for example, modifications that introduce nucleic acid molecules (which may include an expression control element, such as a promoter) that encode one or more proteins or enzymes, or other nucleic acid molecule additions, deletions, substitutions, or other functional disruption of or addition to a cell's genetic material. Exemplary modifications include those in coding regions or functional fragments thereof of heterologous or homologous polypeptides from a reference or parent molecule.

A “conservative substitution” is recognized in the art as a substitution of one amino acid for another amino acid that has similar properties. Exemplary conservative substitutions are well known in the art (see, e.g., WO 97/09433 at page 10; Lehninger, Biochemistry, 2^{nd} Edition; Worth Publishers, Inc. NY, NY, pp. 71-77, 1975; Lewin, Genes IV, Oxford University Press, NY and Cell Press, Cambridge, MA, p. 8, 1990).

### Binding Proteins

A “binding protein” as used herein, refers to a proteinaceous molecule or portion thereof (e.g., peptide, oligopeptide, polypeptide, protein) that possesses the ability to specifically and non-covalently associate, unite, or combine with a target (e.g., a protein, peptide or fragment thereof, peptide-MHC complex). A binding protein may be purified, substantially purified, synthetic or recombinant. Exemplary binding proteins include single chain immunoglobulin variable regions (e.g., scTCR, scFv).

In certain embodiments, any of the binding proteins of the invention are each a T cell receptor (TCR), a chimeric antigen receptor or an antigen-binding fragment of a TCR, any of which can be chimeric, humanized or human. In any embodiment, the binding protein may be a chimeric or fusion protein. In further embodiments, an antigen-binding fragment of the TCR comprises a single chain TCR (scTCR) or a chimeric antigen receptor (CAR). In certain embodiments, a binding protein is a TCR.

“T cell receptor” (TCR) refers to an immunoglobulin superfamily member (having a variable binding domain, a constant domain, a transmembrane region, and a short cytoplasmic tail; see, e.g., Janeway et al., Immunobiology: The Immune System in Health and Disease, 3^{rd} Ed., Current Biology Publications, p. 4:33, 1997) capable of specifically binding to an antigen peptide bound to a MHC receptor. A TCR can be found on the surface of a cell or in soluble form and generally is comprised of a heterodimer having α (alpha) and β (beta) chains (also known as TCRα and TCRβ, respectively), or γ and δ chains (also known as TCRγ and TCRδ, respectively). Like immunoglobulins, the extracellular portion of TCR chains (e.g., α-chain, β-chain) contain two immunoglobulin domains, a variable domain (e.g., α-chain variable domain or Vα, β-chain variable domain or VP; typically amino acids 1 to 116 based on Kabat numbering Kabat et al., “Sequences of Proteins of Immunological Interest, US Dept. Health and Human Services, Public Health Service National Institutes of Health, 1991, 5^{th} ed.) at the N-terminus, and one constant domain (e.g., α-chain constant domain or C_{α}, typically amino acids 117 to 259 based on Kabat, β-chain constant domain or Cβ, typically amino acids 117 to 295 based on Kabat) adjacent to the cell membrane. Also like immunoglobulins, the variable domains contain complementary determining regions (CDRs) separated by framework regions (FRs) (see, e.g., Jores et al., Proc. Nat'l. Acad. Sci. U.S.A. 57:9138, 1990; Chothia et al, EMBO J. 7:3745, 1988; see also Lefranc et al., Dev. Comp. Immunol. 27:55, 2003). In certain embodiments, a TCR is found on the surface of T cells (or T lymphocytes) and associates with the CD3 complex. The source of a TCR as used in the present disclosure may be from various animal species, such as a human, mouse, rat, rabbit or other mammal. The TCR referred to herein may be cell bound TCR or a soluble TCR. The cell bound TCR or soluble TCR may be part of a chimeric or fusion protein.

For MHCI, pockets A-F are defined in Young et al., FASEB J (1995): 9, 26-36. For MHCII, pockets 1 to 9 in MHCII are named after the residue of the peptide that they contain, see for example, Rammensee et al. Curr Opin Immunol (1995): 7: 85-96.

Methods useful for isolating and purifying recombinantly produced soluble TCR (also when part of a chimeric or fusion protein), by way of example, may include obtaining supernatants from suitable host cell/vector systems that secrete the recombinant soluble TCR into culture media and then concentrating the media using a commercially available filter. Following concentration, the concentrate may be applied to a single suitable purification matrix or to a series of suitable matrices, such as an affinity matrix or an ion exchange resin. One or more reverse phase HPLC steps may be employed to further purify a recombinant polypeptide. These purification methods may also be employed when isolating an immunogen from its natural environment. Methods for large scale production of one or more of the isolated/recombinant soluble TCR described herein include batch cell culture, which is monitored and controlled to maintain appropriate culture conditions. Purification of the soluble TCR may be performed according to methods described herein and known in the art.

The binding proteins or domains as described herein may be functionally characterized according to any of a large number of art accepted methodologies for assaying T cell activity, including determination of T cell binding, activation or induction and also including determination of T cell responses that are antigen-specific. Examples include determination of T cell proliferation, T cell cytokine release, antigen specific T cell stimulation, MHC restricted T cell stimulation, CTL activity (e.g., by detecting Cr release from pre-loaded target cells), changes in T cell phenotypic marker expression, and other measures of T-cell functions. Procedures for performing these and similar assays may be found, for example, in Lefkovits (Immunology Methods Manual: The Comprehensive Sourcebook of Techniques, 1998). See, also, Current Protocols in Immunology; Weir, Handbook of Experimental Immunology, Blackwell Scientific, Boston, MA (1986); Mishell and Shigii (eds.) Selected Methods in Cellular Immunology, Freeman Publishing, San Francisco, CA (1979); Green and Reed, Science 281:1309 (1998) and references cited therein.

As used herein, MHC and HLA are used interchangeably, and all instances of HLA may be substituted with MHC. The mouse MHC is also called H2.

In any aspect or embodiment, the cysteine is introduced into the CDR (e.g. of the variable domain of the binding protein, or chimeric or fusion protein) by mutation or modification of an existing residue. In other words, the native amino acid at a particular position may not be cysteine but the native amino acid is mutated to a cysteine residue or modified in any way to allow that residue to form a disulphide bond with a cysteine present in a peptide bound to a HLA molecule. Exemplary methods are described herein, including the Examples, which describe mutating a nucleotide sequence encoding a variable domain of the binding protein, or chimeric or fusion protein at a relevant position to encode a cysteine residue. Where a cysteine has been introduced, eg. by way of mutation or modification, the cysteine may be referred to as a non-native cysteine, and any binding protein, chimeric or fusion protein, containing an introduced cysteine may be referred to as a binding protein, chimeric or fusion protein comprising a non-native cysteine.

A protein disulphide bond is a covalent link between the sulphur atoms of the thiol groups (—SH) in two cysteine residues. The disulphide (also called an S-S bond, disulphide bridge, or crosslink) is formed upon oxidation of the two thiols, thus linking the two cysteines and their respective main peptide chains by the covalent disulphide bond.

The binding protein may comprise any amino acid sequence, for example any CDR1, CDR2 or CDR3 amino acid sequence of a Vα and/or Vβ, as described herein, including the Examples and FIG. 17. Preferably, the binding protein contains a cysteine residue introduced into the CDR by mutation or modification of an existing residue.

### Adoptive Cell Therapy/Engineered Cells

The present invention provides methods of preparing cells for adoptive cell therapy, methods of treating or preventing a disease or condition as described herein in subjects with those cells and the cells per se.

In certain embodiments, nucleic acid molecules encoding a binding protein, or chimeric or fusion protein, of the invention are used to transfect/transduce a host cell (e.g., CD8+ T cells, Treg cells) for use in adoptive transfer therapy.

In alternative embodiments, one or more peptides of the invention are used to activate and/or expand a population of T cells, in order to generate T cells (e.g., CD8+ T cells, Treg cells) having specificity for the peptide.

Advances in TCR sequencing have been described (e.g., Robins et al, Blood 114:4099, 2009; Robins et al, Sci. Translat. Med. 2:47ra64, 2010; Robins et al, (September 10) J. 1 mm. Meth. Epub ahead of print, 2011; Warren et al, Genome Res. 2 1:790, 2011) and may be employed in the course of practicing the embodiments according to the present disclosure. Similarly, methods for transfecting/transducing T cells with desired nucleic acids have been described (e.g., U.S. Patent Application Pub. No. US 2004/0087025) as have adoptive transfer procedures using T cells of desired antigen-specificity (e.g., Schmitt et al, Hum. Gen. 20:1240, 2009; Dossett et al, Mol. Ther. 77:742, 2009; Till et al, Blood 772:2261, 2008; Wang et al, Hum. Gene Ther. 75:712, 2007; Kuball et al, Blood 709:2331, 2007; US 2011/0243972; US 2011/0189141; e n et al, Ann. Rev. Immunol. 25:243, 2007), such that adaptation of these methodologies to the presently disclosed embodiments is contemplated, based on the teachings herein, including those directed to binding proteins of the invention.

The T cells may be selected from the group consisting of tumour infiltrating lymphocytes, peripheral blood lymphocytes, genetically engineered to express T cell receptors or chimeric antigen receptors (CARs), γδ T cells, enriched with mixed lymphocyte tumour cell cultures (MLTCs) or cloned using autologous antigen presenting cells and tumour derived peptides. The lymphocytes may be isolated from a histocompatible donor, or from the subject.

CD8+ T cells may be obtained using routine cell sorting techniques that discriminate and segregate T cells based on T cell surface markers and can be used to obtain an isolated population of CD8+ T cells for use in the compositions and methods of the invention. For example, a biological sample including blood and/or peripheral blood lymphocytes can be obtained from an individual and CD8+ T cells isolated from the sample using commercially available devices and reagents, thereby obtaining an isolated population of CD8+ T cells.

Human CD8+ T-cell types and/or populations can be identified using the phenotypic cell-surface markers CD62L, CCR7, CD27, CD28 and CD45RA or CD45RO. As used herein, CD8+ T-cell types and/or populations have the following characteristics or pattern of expression of cell surface markers: Naïve T cells are characterized as CD45RA+, CD27+, CD28+, CD62L+ and CCR7+; CD45RO+ Central Memory T cells are CD45RA−, CD27+, CD28+, CD62L+ and CCR7+; CD45RO+ Effector Memory T cells are defined by the lack of expression of these five markers (CD45RA−, CD27−, CD28−, CD62L− and CCR7−); and terminally differentiated Effector Memory CD45RA+ T cells are characterized as CD45RA+, CCR7−, CD27−, CD28−, CD62L−. Terminally differentiated Effector Memory cells further up-regulate markers such as CD57, KLRG1, CX3CR1 and exhibit strong cytotoxic properties characterized by their ability to produce high levels of Granzyme A and B, Perforin and IFNγ. Therefore, various populations of T cells can be separated from other cells and/or from each other based on their expression or lack of expression of these markers.

Different CD8+ T cell types can also exhibit particular functions, including, for example: secretion of IFN-γ; secretion of IL-2; production of Granzyme B; expression of FasL and expression of CD 107. However, while the expression pattern of cell surface markers is considered diagnostic of each particular CD8+ T cell type and/or population as described herein, the functional attributes of each cell type and/or population may vary depending on the amount of stimulation the cell(s) has or have received.

A population of cells comprising cytotoxic or regulatory T (Treg) cells may be derived from any source in which cytotoxic or Treg cells exist, such as peripheral blood, the thymus, lymph nodes, spleen, and bone marrow.

A population of cells comprising Treg cells may also be derived from a mixed population of T cells, or from a population of conventional T cells. As described herein, the mixed population or conventional T cells may be contacted with a desired peptide/MHC ligand to enrich antigen specificity in the T cells. Alternatively, the mixed population or conventional T cells may be transduced with a nucleic acid encoding a binding protein of the invention. The T cells may then be converted into Treg cells using standard techniques known to the skilled person for generation of Treg cells. In certain embodiments, the mixed population of T cells, or conventional T cells are cultured in conditions to allow for increased expression of TGF-beta, and Foxp3. This includes culturing cells with anti-CD3/anti-CD28 antibodies, inhibition of CDK8/19, high doses of IL-2, TGF-beta, and rapamycin. In further embodiments, the converted or enriched population of Treg cells are stabilised (for example, by contacting the cells with Vitamin C or other agent for stabilising the Tregs).

The Treg cells used for infusion (or indeed the Tconv or mixed population of T cells used to generate the Tregs) can be isolated from an allogenic donor, preferably HLA matched, or from the subject diagnosed with a condition associated with the aberrant, unwanted or otherwise inappropriate immune response to a self-protein.

The T cells may also be generated from differentiation of induced pluripotent cells (iPSCs) or embryonic stem cells, preferably an embryonic stem cell line. The skilled person will be familiar with standard techniques for generating Treg cells from a stem cells, including an iPSC. Examples of these techniques are described in: Hague et al., (2012) J. Immunol., 189: 2338-36; and Hague et al., (2019) JCI Insight, 4: pii 126471).

Further still, in the context of a mixed population of T cells, the skilled person will be familiar with standard techniques for isolating the subpopulation of the T cells which are CD4+CD25+ T cells (Treg cells). For example, CD4+CD25+ T cells (Treg cells) can be obtained from a biological sample from a subject by negative and positive immuno-selection and cell sorting.

In any method of the invention the Treg cells that have been cultured in the presence of a nucleic acid or vector can be transferred into the same subject from which cells were obtained. In other words, the cells used in a method of the invention can be an autologous cell, i.e., can be obtained from the subject in which the medical condition is treated or prevented. Alternatively, the cell can be allogenically transferred into another subject. Preferably, the cell is autologous to the subject in a method of treating or preventing a medical condition in the subject.

As used herein, the term “ex vivo” or “ex vivo therapy” refers to a therapy where cells are obtained from a patient or a suitable alternate source, such as, a suitable allogenic donor, and are modified, such that the modified cells can be used to treat a disease which will be improved by the therapeutic benefit produced by the modified cells. Treatment includes the administration or re-introduction of the modified cells into the patient. A benefit of ex vivo therapy is the ability to provide the patient the benefit of the treatment, without exposing the patient to undesired collateral effects from the treatment.

The term “administered” means administration of a therapeutically effective dose of the aforementioned composition including the respective cells to an individual. By “therapeutically effective amount” is meant a dose that produces the effects for which it is administered. The exact dose will depend on the purpose of the treatment, and will be ascertainable by one skilled in the art using known techniques. As is known in the art and described above, adjustments for systemic versus localized delivery, age, body weight, general health, sex, diet, time of administration, drug interaction and the severity of the condition may be necessary, and will be ascertainable with routine experimentation by those skilled in the art.

An “enriched” or “purified” population of cells is an increase in the ratio of particular cells to other cells, for example, in comparison to the cells as found in a subject's body, or in comparison to the ratio prior to exposure to a peptide, nucleic acid or vector of the invention. In some embodiments, in an enriched or purified population of cells, the particular cells include at least 20%, 30%, 40%, 50%, 60%, 70%, 75%, 80%, 90%, 95% or 99% of the total cell population. A population of cells may be defined by one or more cell surface markers and/or properties.

Treg cells that express a binding protein, or chimeric or fusion protein, of the invention can be administered to the subject by any method including, for example, injection, infusion, deposition, implantation, oral ingestion, or topical administration, or any combination thereof. Injections can be, e.g., intravenous, intramuscular, intradermal, subcutaneous or intraperitoneal, preferably intravenous. Single or multiple doses can be administered over a given time period, depending upon the condition, the severity thereof and the overall health of the subject, as can be determined by one skilled in the art without undue experimentation. The injections can be given at multiple locations.

Administration of the Treg cells can be alone or in combination with other therapeutic agents. Each dose can include about 10×10³ CD8+ T cells, 20×10³ cells, 50×10³ cells, 100×10³ cells, 200×10³ cells, 500×10³ cells, 1×10⁶ cells, 2×10⁶ cells, 20×10⁶ cells, 50×10⁶ cells, 100×10⁶ cells, 200×10⁶, 500×10⁶, 1×10⁹ cells, 2×10⁹ cells, 5×10⁹ cells, 10×10⁹ cells, and the like. Administration frequency can be, for example, once per week, twice per week, once every two weeks, once every three weeks, once every four weeks, once per month, once every two months, once every three months, once every four months, once every five months, once every six months, and so on. The total number of days where administration occurs can be one day, on 2 days, or on 3, 4, 5, 6, 7, 8, 9, 10, 11, 12, 13, 14, 15, 16, 17, 18, 19, or 20 days, and so on. It is understood that any given administration might involve two or more injections on the same day. For administration, at least 20%, at least 30%, at least 40%, at least 50%, at least 60%, at least 70%, at least 80%, at least 90%, at least 95%, at least 99%, of the Treg cells that are administered exhibit at least one property of a Treg cell.

### Peptides

As described herein, the binding proteins, chimeric of fusion proteins, of the invention may bind to peptides that are bound to an MHC molecule, preferably, a HLA molecule. The peptides may be endogenous or exogenous, self or non-self. The peptides include any peptides referred to herein with a cysteine residue present or when a cysteine has been introduced at a specified position that allows formation of a disulphide bond with a cysteine in a binding protein of the invention.

A low abundant peptide is a peptide which fails to detectably affect the number, or differentiation state, of T cells that bind specifically to the peptide/MHC complex. The peptide may be endogenous or exogenous. The endogenous peptide may be any described herein including tumour antigens. The T cell population may be described as “ignorant” of low abundant peptides. In the thymus of mice, recognised low abundant peptides were found to be expressed in 0.003%-0.015% of EpCAM+ epithelial cells and 0.0003%-0.001% of dendritic cells.

MHC class I and II proteins are important in the adaptive immune system. Both classes of proteins share the task of presenting peptides on the surface for recognition by T cells. MHC class I peptide complexes are presented on nucleated cells and are recognised by cytotoxic CD8+ T cells. In contrast, MHC class II peptide complexes are on the surface of professional antigen-presenting cells, such as dendritic cells, macrophages or B cells, and act to activate CD4+ T cells, which results in coordination and regulation of effector functions.

Major histocompatibility complex class I and class II share an overall similar folding. The binding platform is composed of two domains, originating from a single heavy α-chain (HC) in the case of MHC class I and from two chains in the case of MHC class II (α-chain and β-chain). The two domains have a curved β-sheet as a base and two α-helices on top, which are far enough apart to accommodate a peptide chain in-between. Two membrane-proximal immunoglobulin (Ig) domains support the peptide-binding unit. One Ig domain is present in each chain of MHC class II, while the second Ig-type domain of MHC class I is provided by non-covalent association of the invariant light chain beta-2 microglobulin (β2m) with the HC. Transmembrane helices anchor the HC of MHC class I and both chains of MHC class II in the membrane.

The groove in-between the two helices accommodates peptides based on (i) the formation of a set of conserved hydrogen bonds between the side-chains of the MHC molecule and the backbone of the peptide and (ii) the occupation of defined pockets by peptide side chains (anchor residues P2 or P5/6 and PQ in MHC class I and P1, P4, P6, and P9 in MHC class II). The type of interactions of individual peptide side-chains with the MHC depend on the geometry, charge distribution, and hydrophobicity of the binding groove.

Non-limiting examples of HLA class I molecules include HLA-C*07:01, HLA-A*2402, HLA-A*2, HLA-A*24, HLA-A*02:01, HLA-B*07:02, HLA-B*44:05, HLA-B*35:01, HLA-A*01:01, HLA-B*37:01, HLA-B*08:01, HLA-A*11:01, HLA-B*08:01, HLA-B*27:05, HLA-B*35:08, HLA-A*24:02, HLA-B*51:01, and HLA-E*01:03.

Non-limiting examples of HLA class II molecules include HLA-DQA1*0508_HLA-DQB1*0201, HLA-DQA1*0501_HLA-DQB1*0201, HLA-DQA1*0301_HLA-DQB1*0302, HLA-DRA*0101_HLA-DBR1*0101, HLA-DRA*0101_HLA-DRB3*0301, HLA-DRA*0101_HLA-DBR5*0101, HLA-DRA*0101_HLA-DBR1*0401, HLA-DPA1*0103_HLA-DPB1*2602, HLA-DQA1*0501_HLA-DQB1*0302, HLA-DRA*0101_HLA-DRB1*1101, HLA-DRA*0101_HLA-DRB1*1502, HLA-DRA*0101_HLA-DRB1*0101, HLA-DQA1*0301_HLA-DQB1*0305, HLA-DQA1*0201_HLA-DQB1*0201.

Reference to a “peptide” includes reference to a peptide, polypeptide or protein or parts thereof. The peptide may be glycosylated or unglycosylated and/or may contain a range of other molecules fused, linked, bound or otherwise associated to the protein such as amino acids, lipids, carbohydrates or other peptides, polypeptides or proteins. Reference hereinafter to a “peptide” includes a peptide comprising a sequence of amino acids as well as a peptide associated with other molecules such as amino acids, lipids, carbohydrates or other peptides, polypeptides or proteins.

“Derivatives” include fragments, parts, portions and variants from natural, synthetic or recombinant sources including fusion proteins. Parts or fragments include, for example, active regions of the subject peptide. Derivatives may be derived from insertion, deletion or substitution of amino acids. Amino acid insertional derivatives include amino and/or carboxylic terminal fusions as well as intrasequence insertions of single or multiple amino acids. Insertional amino acid sequence variants are those in which one or more amino acid residues are introduced into a predetermined site in the protein although random insertion is also possible with suitable screening of the resulting product. Deletional variants are characterized by the removal of one or more amino acids from the sequence.

Substitutional amino acid variants are those in which at least one residue in the sequence has been removed and a different residue inserted in its place. An example of substitutional amino acid variants are conservative amino acid substitutions. Conservative amino acid substitutions typically include substitutions within the following groups: glycine and alanine; valine, isoleucine and leucine; aspartic acid and glutamic acid; asparagine and glutamine; serine and threonine; lysine and arginine; and phenylalanine and tyrosine. Additions to amino acid sequences include fusions with other peptides, polypeptides or proteins. In one embodiment, cysteine residues are substituted with serine, as exemplified herein.

Chemical and functional equivalents of the subject peptide should be understood as molecules exhibiting any one or more of the functional activities of these molecules and may be derived from any source such as being chemically synthesized or identified via screening processes such as natural product screening.

Analogues contemplated herein include, but are not limited to, modification to side chains, incorporating of unnatural amino acids and/or their derivatives during peptide, polypeptide or protein synthesis and the use of crosslinkers and other methods which impose conformational constraints on the proteinaceous molecules or their analogues.

Examples of side chain modifications contemplated by the present invention include modifications of amino groups such as by reductive alkylation by reaction with an aldehyde followed by reduction with NaBH₄; amidination with methylacetimidate; acylation with acetic anhydride; carbamoylation of amino groups with cyanate; trinitrobenzylation of amino groups with 2, 4, 6-trinitrobenzene sulphonic acid (TNBS); acylation of amino groups with succinic anhydride and tetrahydrophthalic anhydride; and pyridoxylation of lysine with pyridoxal-5-phosphate followed by reduction with NaBH₄.

The guanidine group of arginine residues may be modified by the formation of heterocyclic condensation products with reagents such as 2,3-butanedione, phenylglyoxal and glyoxal. The carboxyl group may be modified by carbodiimide activation via O-acylisourea formation followed by subsequent derivatisation, for example, to a corresponding amide. Sulphydryl groups may be modified by methods such as carboxymethylation with iodoacetic acid or iodoacetamide; performic acid oxidation to cysteic acid; formation of mixed disulphides with other thiol compounds; reaction with maleimide, maleic anhydride or other substituted maleimide; formation of mercurial derivatives using 4-chloromercuribenzoate, 4-chloromercuriphenylsulphonic acid, phenylmercury chloride, 2-chloromercuri-4-nitrophenol and other mercurials; carbamoylation with cyanate at alkaline pH. Tryptophan residues may be modified by, for example, oxidation with N-bromosuccinimide or alkylation of the indole ring with 2-hydroxy-5-nitrobenzyl bromide or sulphenyl halides. Tyrosine residues on the other hand, may be altered by nitration with tetranitromethane to form a 3-nitrotyrosine derivative.

Modification of the imidazole ring of a histidine residue may be accomplished by alkylation with iodoacetic acid derivatives or N-carboethoxylation with diethylpyrocarbonate.

Examples of incorporating unnatural amino acids and derivatives during protein synthesis include, but are not limited to, use of norleucine, 4-amino butyric acid, 4-amino-3-hydroxy-5-phenylpentanoic acid, 6-aminohexanoic acid, t-butylglycine, norvaline, phenylglycine, ornithine, sarcosine, 4-amino-3-hydroxy-6-methylheptanoic acid, 2-thienyl alanine and/or D-isomers of amino acids.

Crosslinkers can be used, for example, to stabilise 3D conformations, using homo-bifunctional crosslinkers such as the bifunctional imido esters having (CH₂)ₙ spacer groups with n=1 to n=6, glutaraldehyde, N-hydroxysuccinimide esters and hetero-bifunctional reagents which usually contain an amino-reactive moiety such as N-hydroxysuccinimide and another group specific-reactive moiety.

It is possible to modify the structure of a peptide according to the invention for various purposes such as for increasing solubility, enhancing therapeutic or preventative efficacy, enhancing stability or increasing resistance to proteolytic degradation. A modified peptide may be produced in which the amino acid sequence has been altered, such as by amino acid substitution, deletion or addition, to modify immunogenicity. Similarly, components may be added to peptides of the invention to produce the same result.

For example, a peptide can be modified so that it exhibits the ability to induce T cell anergy. In this instance, critical binding residues for the T cell receptor can be determined using known techniques (for example substitution of each residue and determination of the presence or absence of T cell reactivity) In one example, those residues shown to be essential to interact with the T cell receptor can be modified by replacing the essential amino acid with another, preferably similar amino acid residue (a conservative substitution) whose presence is shown to alter T cell reactivity or T cell functioning. In addition, those amino acid residues which are not essential for T cell receptor interaction can be modified by being replaced by another amino acid whose incorporation may then alter T cell reactivity or T cell functioning but does not, for example, eliminate binding to relevant MHC proteins.

Exemplary conservative substitutions are detailed, below, and include:

| Original Residue | Exemplary Substitutions |
|---|---|
| Ala | Ser |
| Arg | Lys |
| Asn | Gln, His |
| Asp | Glu |
| Cys | Ser, Ala |
| Gln | Asn |
| Glu | Asp |
| Gly | Pro |
| His | Asn, Gln |
| Ile | Leu, Val |
| Leu | Ile, Val |
| Lys | Arg, Gln, Glu |
| Met | Leu, Ile |
| Phe | Met, Leu, Tyr |
| Ser | Thr |
| Thr | Ser |
| Trp | Tyr |
| Tyr | Trp, Phe |
| Val | Ile, Leu |

Such modifications will result in the production of molecules falling within the scope of “mutants” of the subject peptide as herein defined. “Mutants” should be understood as a reference to peptides which exhibit one or more structural features or functional activities which are distinct from those exhibited by the non-mutated peptide counterpart.

Peptides of the invention may also be modified to incorporate one or more polymorphisms resulting from natural allelic variation and D-amino acids, non-natural amino acids or amino acid analogues may be substituted into the peptides to produce modified peptides which fall within the scope of the invention. Peptides may also be modified by conjugation with polyethylene glycol (PEG) by known techniques. Reporter groups may also be added to facilitate purification and potentiality increase solubility of the peptides according to the invention. Other well known types of modification including insertion of specific endoprotease cleavage sites, addition of functional groups or replacement of hydrophobic residues with less hydrophobic residues as well as site-directed mutagenesis of DNA encoding the peptides of the invention may also be used to introduce modifications which could be useful for a wide range of purposes. The various modifications to peptides according to the invention which have been mentioned above are mentioned by way of example only and are merely intended to be indicative of the broad range of modifications which can be effected.

### Nucleic Acids and Vectors

In another aspect, the present invention provides a nucleic acid molecule composition comprising one or more nucleic acid molecules encoding or complementary to a sequence encoding the binding proteins, or chimeric or fusion proteins, and peptides of the invention or a derivative, homologue or analogue thereof. The nucleic acid molecules of the invention may be used to produce a binding protein, chimeric or fusion protein, or peptide of the invention, or used for cell therapy to treat a disease or condition described herein.

The term “construct” refers to any polynucleotide that contains a recombinant nucleic acid molecule. A construct may be present in a vector (e.g., a bacterial vector, a viral vector) or may be integrated into a genome. A “vector” is a nucleic acid molecule that is capable of transporting another nucleic acid molecule. Vectors may be, for example, plasmids, cosmids, viruses, a RNA vector or a linear or circular DNA or RNA molecule that may include chromosomal, non-chromosomal, semi-synthetic or synthetic nucleic acid molecules. Exemplary vectors are those capable of autonomous replication (episomal vector) or expression of nucleic acid molecules to which they are linked (expression vectors).

Viral vectors include retrovirus, adenovirus, parvovirus (e.g., adeno-associated viruses), coronavirus, negative strand RNA viruses such as ortho-myxovirus (e.g., influenza virus), rhabdovirus (e.g., rabies and vesicular stomatitis virus), paramyxovirus (e.g., measles and Sendai), positive strand RNA viruses such as picornavirus and alphavirus, and double-stranded DNA viruses including adenovirus, herpesvirus (e.g., Herpes Simplex virus types 1 and 2, Epstein-Barr virus, cytomegalovirus), and poxvirus (e.g., vaccinia, fowlpox and canarypox). Other viruses include Norwalk virus, togavirus, flavivirus, reoviruses, papovavirus, hepadnavirus, and hepatitis virus, for example. Examples of retroviruses include avian leukosis-sarcoma, mammalian C-type, B-type viruses, D type viruses, HTLV-BLV group, lentivirus, spumavirus (Coffin, J. M., Retroviridae: The viruses and their replication, In Fundamental Virology, Third Edition, B. N. Fields et al., Eds., Lippincott-Raven Publishers, Philadelphia, 1996).

“Lentiviral vector,” as used herein, means HIV-based lentiviral vectors for gene delivery, which can be integrative or non-integrative, have relatively large packaging capacity, and can transduce a range of different cell types. Lentiviral vectors are usually generated following transient transfection of three (packaging, envelope and transfer) or more plasmids into producer cells. Like HIV, lentiviral vectors enter the target cell through the interaction of viral surface glycoproteins with receptors on the cell surface. On entry, the viral RNA undergoes reverse transcription, which is mediated by the viral reverse transcriptase complex. The product of reverse transcription is a double-stranded linear viral DNA, which is the substrate for viral integration into the DNA of infected cells.

The term “operably-linked” refers to the association of two or more nucleic acid molecules on a single nucleic acid fragment so that the function of one is affected by the other. For example, a promoter is operably-linked with a coding sequence when it is capable of affecting the expression of that coding sequence (i.e., the coding sequence is under the transcriptional control of the promoter). “Unlinked” means that the associated genetic elements are not closely associated with one another and the function of one does not affect the other.

As used herein, “expression vector” refers to a DNA construct containing a nucleic acid molecule that is operably-linked to a suitable control sequence capable of effecting the expression of the nucleic acid molecule in a suitable host. Such control sequences include a promoter to effect transcription, an optional operator sequence to control such transcription, a sequence encoding suitable mRNA ribosome binding sites, and sequences which control termination of transcription and translation. The vector may be a plasmid, a phage particle, a virus, or simply a potential genomic insert. Once transformed into a suitable host, the vector may replicate and function independently of the host genome, or may, in some instances, integrate into the genome itself. In the present specification, “plasmid,” “expression plasmid,” “virus” and “vector” are often used interchangeably.

The term “expression”, as used herein, refers to the process by which a polypeptide is produced based on the encoding sequence of a nucleic acid molecule, such as a gene. The process may include transcription, post-transcriptional control, post-transcriptional modification, translation, post-translational control, post-translational modification, or any combination thereof.

The term “introduced” in the context of inserting a nucleic acid molecule into a cell, means “transfection”, or “transformation” or “transduction” and includes reference to the incorporation of a nucleic acid molecule into a eukaryotic or prokaryotic cell wherein the nucleic acid molecule may be incorporated into the genome of a cell (e.g., chromosome, plasmid, plastid, or mitochondrial DNA), converted into an autonomous replicon, or transiently expressed (e.g., transfected mRNA).

As used herein, “heterologous” or “exogenous” nucleic acid molecule, construct or sequence refers to a nucleic acid molecule or portion of a nucleic acid molecule that is not native to a host cell, but may be homologous to a nucleic acid molecule or portion of a nucleic acid molecule from the host cell. The source of the heterologous or exogenous nucleic acid molecule, construct or sequence may be from a different genus or species. In certain embodiments, a heterologous or exogenous nucleic acid molecule is added (i.e., not endogenous or native) to a host cell or host genome by, for example, conjugation, transformation, transfection, electroporation, or the like, wherein the added molecule may integrate into the host genome or exist as extra-chromosomal genetic material (e.g., as a plasmid or other form of self-replicating vector), and may be present in multiple copies. In addition, “heterologous” refers to a non-native enzyme, protein or other activity encoded by an exogenous nucleic acid molecule introduced into the host cell, even if the host cell encodes a homologous protein or activity.

As described herein, more than one heterologous or exogenous nucleic acid molecule can be introduced into a host cell as separate nucleic acid molecules, as a plurality of individually controlled genes, as a polycistronic nucleic acid molecule, as a single nucleic acid molecule encoding a fusion protein, or any combination thereof. For example, as disclosed herein, a host cell can be modified to express two or more heterologous or exogenous nucleic acid molecules encoding desired TCR specific for a WT-1 antigen peptide (e.g., TCRα and TCR-β). When two or more exogenous nucleic acid molecules are introduced into a host cell, it is understood that the two or more exogenous nucleic acid molecules can be introduced as a single nucleic acid molecule (e.g., on a single vector), on separate vectors, integrated into the host chromosome at a single site or multiple sites, or any combination thereof. The number of referenced heterologous nucleic acid molecules or protein activities refers to the number of encoding nucleic acid molecules or the number of protein activities, not the number of separate nucleic acid molecules introduced into a host cell.

As used herein, the term “endogenous” or “native” refers to a gene, protein, or activity that is normally present in a host cell. Moreover, a gene, protein or activity that is mutated, overexpressed, shuffled, duplicated or otherwise altered as compared to a parent gene, protein or activity is still considered to be endogenous or native to that particular host cell. For example, an endogenous control sequence from a first gene (e.g., promoter, translational attenuation sequences) may be used to alter or regulate expression of a second native gene or nucleic acid molecule, wherein the expression or regulation of the second native gene or nucleic acid molecule differs from normal expression or regulation in a parent cell.

The term “homologous” or “homolog” refers to a molecule or activity found in or derived from a host cell, species or strain. For example, a heterologous or exogenous nucleic acid molecule may be homologous to a native host cell gene, and may optionally have an altered expression level, a different sequence, an altered activity, or any combination thereof.

“Sequence identity,” as used herein, refers to the percentage of amino acid residues in one sequence that are identical with the amino acid residues in another reference polypeptide sequence after aligning the sequences and introducing gaps, if necessary, to achieve the maximum percent sequence identity, and not considering any conservative substitutions as part of the sequence identity. The percentage sequence identity values can be generated using the NCBI BLAST2.0 software as defined by Altschul et al. (1997) “Gapped BLAST and PSI-BLAST: a new generation of protein database search programs”, Nucleic Acids Res. 25:3389-3402, with the parameters set to default values.

As used herein, the term “host” refers to a cell (e.g., Treg cell) or microorganism targeted for genetic modification with a heterologous or exogenous nucleic acid molecule to produce a polypeptide of interest (e.g., high or enhanced affinity anti-WT-1 TCR). In certain embodiments, a host cell may optionally already possess or be modified to include other genetic modifications that confer desired properties related or unrelated to biosynthesis of the heterologous or exogenous protein (e.g., inclusion of a detectable marker; deleted, altered or truncated endogenous TCR; increased co-stimulatory factor expression). In some embodiments, host cells are genetically modified to express a protein or fusion protein that modulates immune signaling in a host cell to, for example, promote survival and/or expansion advantage to the modified cell (e.g., see immunomodulatory fusion proteins of WO 2016/141357, which are herein incorporated by reference in their entirety).

The nucleic acid molecule may be ligated to an expression vector capable of expression in a prokaryotic cell (e.g., E. coli) or a eukaryotic cell (e.g., yeast cells, fungal cells, insect cells, mammalian cells or plant cells). The nucleic acid molecule may be ligated or fused or otherwise associated with a nucleic acid molecule encoding another entity such as, for example, a signal peptide. It may also comprise additional nucleotide sequence information fused, linked or otherwise associated with it either at the 3′ or 5′ terminal portions or at both the 3′ and 5′ terminal portions. The nucleic acid molecule may also be part of a vector, such as an expression vector. The latter embodiment facilitates production of recombinant forms of the binding protein or peptide of the present invention.

Such nucleic acids may be useful for recombinant production of binding proteins or peptides of the invention or proteins comprising them by insertion into an appropriate vector and transfection into a suitable cell line. Such expression vectors and host cell lines also form an aspect of the invention.

In producing peptides by recombinant techniques, host cells transformed with a nucleic acid having a sequence encoding a binding protein, chimeric or fusion protein, or peptide according to the invention or a functional equivalent of the nucleic acid sequence are cultured in a medium suitable for the particular cells concerned. Binding proteins, chimeric or fusion proteins, or peptides can then be purified from cell culture medium, the host cells or both using techniques well known in the art such as ion exchange chromatography, gel filtration chromatography, ultrafiltration, electrophoresis or immunopurification with antibodies specific for the binding protein or peptide.

Nucleic acids encoding binding proteins or peptides of the invention may be expressed in bacterial cells such as E. coli, insect cells, yeast or mammalian cells such as Chinese hamster ovary cells (CHO). Suitable expression vectors, promoters, enhancers and other expression control elements are referred to in Sambruck et al (1989). Other suitable expression vectors, promoters, enhancers and other expression elements are well known to those skilled in the art. Examples of suitable expression vectors in yeast include Yep Sec 1 (Balderi et al., 1987, Embo J., 6:229-234); pMFa (Kurjan and Herskowitz., 1982, Cell., 30:933-943); JRY88 (Schultz et al., 1987, Gene., 54:113-123) and pYES2 (Invitrogen Corporation, San Diego, CA). These vectors are freely available as are baculovirus and mammalian expression systems. For example, a baculovirus system is commercially available (ParMingen, San Diego, CA) for expression in insect cells while the pMsg vector is commercially available (Pharmacia, Piscataway, NJ) for expression in mammalian cells.

For expression in E. coli suitable expression vectors include among others, pTrc (Amann et al., 1998, Gene., 69:301-315) pGex (Amrad Corporation, Melbourne, Australia); pMal (N.E. Biolabs, Beverley, MA); pRit5 (Pharmacia, Piscataway, NJ); pEt-11d (Novagen, Maddison, WI) (Jameel et al., 1990, J. Virol., 64:3963-3966) and pSem (Knapp et al., 1990, Bio Techniques., 8:280-281). The use of pTRC, and pEt-11d, for example, will lead to the expression of unfused protein. The use of pMal, pRit5, pSem and pGex will lead to the expression of a protein or peptide fused to maltose E binding protein (pMal), protein A (pRit5), truncated galactosidase (PSEM) or glutathione S-transferase (pGex). When a binding protein or peptide is expressed as a fusion protein, it is particularly advantageous to introduce an enzymatic cleavage site at the fusion junction between the carrier protein and the peptide concerned. The binding protein or peptide of the invention may then be recovered from the fusion protein through enzymatic cleavage at the enzymatic site and biochemical purification using conventional techniques for purification of proteins and peptides. The different vectors also have different promoter regions allowing constitutive or inducible expression or temperature induction. It may additionally be appropriate to express recombinant peptides in different E. coli hosts that have an altered capacity to degrade recombinantly expressed proteins. Alternatively, it may be advantageous to alter the nucleic acid sequence to use codons preferentially utilised by E. coli, where such nucleic acid alteration would not affect the amino acid sequence of the expressed proteins.

Host cells can be transformed to express the nucleic acids of the invention using conventional techniques such as calcium phosphate or calcium chloride co-precipitation, DEAE-dextran-mediated transfection or electroporation. Suitable methods for transforming the host cells may be found in Sambruck et al. (1989), and other laboratory texts. The nucleic acid sequence of the invention may also be chemically synthesised using standard techniques.

In addition to recombinant production of peptides according to the invention, the nucleic acids may be utilised as probes for experimental or purification purposes.

### Conditions for Treatment

Identification and synthesis of the binding proteins, chimeric or fusion proteins, peptides, cells, nucleic acids, vectors and compositions of the invention as disclosed herein now facilitates the development of a range of prophylactic and therapeutic treatment protocols for use with respect to autoimmune, cancer and infectious diseases. Also facilitated is the development of reagents for use therein. Accordingly, the present invention should be understood to extend to the use of the peptides or functional derivatives, homologues or analogues thereof in the therapeutic and/or prophylactic treatment of patients. Such methods of treatment include, but are not limited to: Infectious diseases and cancers include any referred to in Tables 2, 3 and 4.

Autoimmune diseases include any referred to in Tables 1, 3 and 4.

Transplant rejection and conditions associated with the rejection of a transplant.

Administration to the subject with the binding proteins, chimeric or fusion proteins, peptides, cells, nucleic acids, vectors and compositions of the invention is a means of treating or preventing cancer or an infectious disease. This may be achieved, for example, by introducing a TCR/peptide-MHC class I disulphide bond to increase the cytotoxic activity of CD8 T cells thereby enhancing the anti-tumour (with respect to cancer) or anti-pathogen (with respect to an infectious disease) response. Without being bound by theory, the introduction of a disulphide bond between a CD8 T cell and an antigen presenting cell (any nucleated cell), in the context of MHC class I, can result in greater cytotoxicity or killing of an infected (with respect to infectious disease) or transformed (with respect to cancer) target cell. Alternatively, introduction of a disulphide bond between a CD8 T cell and an antigen presenting cell (any nucleated cell), in the context of MHC class I, can result in greater cytokine/chemokine production resulting in proinflammatory response.

Non-limiting examples of potential cancers that can be treated or prevented by the invention include, melanoma, lung cancer, renal cancer, prostate cancer, breast cancer, colorectal cancer, and fibrosarcoma. Preferably, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin's lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemia, acute myeloid leukemia, chronic myeloid leukemia, acute lymphoblastic leukemia, chronic lymphocytic leukemia, solid tumors of childhood, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers including those induced by asbestos, and combinations of said cancers.

By introducing a TCR/peptide-MHC class I disulphide bond to increase the cytotoxic activity of CD8 T cells, this can be used to treat or prevent infectious diseases. Non-limiting examples of potential infectious diseases that can be treated or prevented by the invention include, Epstein-Barr virus, Influenza, Dengue virus, HIV, Hepatitis C virus, and cytomegalovirus.

Administration to the subject with the binding proteins, chimeric or fusion proteins, peptides, cells, nucleic acids, vectors and compositions of the invention is a means of treating or preventing autoimmune disease. This may be achieved, for example, by introducing a TCR/peptide-MHC class II disulphide bond to increase the regulatory activity of Treg cells thereby inhibiting or suppressing an autoimmune response. Non-limiting examples of potential autoimmune diseases that can be treated or prevented by the invention include, coeliac disease, nickel hypersensitivity, multiple sclerosis, beryllium hypersensitivity, and diabetes.

Administration to the subject with the binding proteins, chimeric or fusion proteins, peptides, cells, nucleic acids, vectors and compositions of the invention is a means of treating or preventing transplant rejection. This may be achieved, for example, by introducing a TCR/peptide-MHC class II disulphide bond to increase the regulatory activity of Treg cells thereby inhibiting or suppressing an immune response directed to the transplant. The treatment includes the treatment of inflammation associated with tissue graft rejection. “Implant rejection” or “graft rejection” means any host-initiated immune response to a graft including, but not limited to, HLA antigens, blood group antigens and the like. Transplant rejection and graft versus host disease can be hyperacute (humoral), acute (T cell mediated), or chronic (unknown etiology), or a combination thereof. Thus, the present invention, is used for the inhibition and/or amelioration of symptoms associated with hyperacute, acute, and/or chronic rejection and/or rejection of any tissue, including, but not limited to, liver, kidney, pancreas, pancreatic islet cells, small intestine, lung, heart, cornea, skin. The graft tissue can be obtained from any donor and can be implanted into any recipient host, or from one part of the body to another.

The phrase “therapeutically effective amount” generally refers to an amount of a cell expressing a binding protein, or peptide of the present invention that (i) treats the particular disease, condition, or disorder, (ii) attenuates, ameliorates, or eliminates one or more symptoms of the particular disease, condition, or disorder, or (iii) delays the onset of one or more symptoms of the particular disease, condition, or disorder described herein.

As used herein, “preventing” or “prevention” is intended to refer to at least the reduction of likelihood of the risk of (or susceptibility to) acquiring a disease or disorder (i.e., causing at least one of the clinical symptoms of the disease not to develop in a individual that may be exposed to or predisposed to the disease but does not yet experience or display symptoms of the disease). Biological and physiological parameters for identifying such patients are provided herein and are also well known by physicians.

In particularly preferred embodiments, the methods of the present invention can be to prevent or reduce the severity, or inhibit or minimise progression, of a flare-up or symptom of a disease or condition as described herein. As such, the methods of the present invention have utility as treatments as well as prophylaxes.

The terms “treatment” or “treating” of a subject includes the purpose of delaying, slowing, stabilizing, curing, healing, alleviating, relieving, altering, remedying, less worsening, ameliorating, improving, or affecting the disease or condition, the symptom of the disease or condition, or the risk of (or susceptibility to) the disease or condition. The term “treating” refers to any indication of success in the treatment or amelioration of infectious disease, cancer or autoimmune disease and associated conditions as herein described, including any objective or subjective parameter such as abatement; remission; lessening of the rate of worsening; lessening severity of the condition; stabilization, diminishing of symptoms or making the condition more tolerable to the individual; slowing in the rate of degeneration or decline; making the final point of degeneration less debilitating; or improving a subject's physical or mental well-being.

It will also be understood that the methods described herein can be used in combination with existing standard of care treatments/therapies for infectious disease, cancer or autoimmune disease.

A “subject” herein is preferably human subject. Although the invention finds application in humans, the invention is also useful for veterinary purposes. The invention is useful for domestic or farm animals such as cattle, sheep, horses and poultry; for companion animals such as cats and dogs; and for zoo animals. It will be understood that the terms “subject” and “individual” are interchangeable in relation to an individual requiring treatment according to the present invention.

### Compositions

Administration of a binding protein, chimeric or fusion protein, peptide, cell, nucleic acid, vector or composition of the present invention (herein referred to as “agent”) in the form of a pharmaceutical composition, may be performed by any convenient means. The agent of the pharmaceutical composition is contemplated to exhibit therapeutic activity when administered in an amount which depends on the particular case. The variation depends, for example, on the human or animal and the agent chosen. A broad range of doses may be applicable. Considering a patient, for example, from about 0.01 μg to about 1 mg of an agent may be administered per dose. Dosage regimens may be adjusted to provide the optimum therapeutic response. For example, several divided doses may be administered daily, weekly, monthly or other suitable time intervals or the dose may be proportionally reduced as indicated by the exigencies of the situation. In another example, said composition is administered initially to induce tolerance and then, if necessary, booster administrations of the composition are administered to maintain tolerance. These boosters may be administered monthly, for example, and may be administered for any period of time, including the life of the patient.

The agent may be administered in a convenient manner such as by the oral, intravenous (where water soluble), intraperitoneal, intramuscular, subcutaneous, intradermal (with or without using a traditional needle or other transdermal delivery device), transdermal, intranasal, sublingual or suppository routes or implanting (e.g. using slow release molecules). Preferably, said composition is administered intradermally. The agent may be administered in the form of pharmaceutically acceptable nontoxic salts, such as acid addition salts or metal complexes, e.g. with zinc, iron or the like (which are considered as salts for purposes of this application). Illustrative of such acid addition salts are hydrochloride, hydrobromide, sulphate, phosphate, maleate, acetate, citrate, benzoate, succinate, malate, ascorbate, tartrate and the like. If the active ingredient is to be administered in tablet form, the tablet may contain a binder such as tragacanth, corn starch or gelatin; a disintegrating agent, such as alginic acid; and a lubricant, such as magnesium stearate. In the context of a peptide for administration, the composition comprising said peptide may be in the form of a liposome or conjugated to nanoparticles. The skilled person will be familiar with standard techniques for formulating peptides for administration to a subject in need thereof.

The pharmaceutical forms suitable for injectable use include sterile aqueous solutions (where water soluble) or dispersions and sterile powders for the extemporaneous preparation of sterile injectable solutions or dispersion or may be in the form of a cream or other form suitable for topical application. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of superfactants. The preventions of the action of microorganisms can be brought about by various antibacterial and antifungal agents, for example, parabens, chlorobutanol, phenol, sorbic acid, thimerosal and the like. Tonicity adjusting agents are useful to keep the preparation isotonic with human plasma and thus avoid tissue damage. Commonly used tonicity agents include Dextrose, Trehalose, Glycerin and Mannitol. Glycerol and sodium chloride are other options but are less commonly used. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilisation. Generally, dispersions are prepared by incorporating the various sterilised active ingredient into a sterile vehicle which contains the basic dispersion medium and the required other ingredients from those enumerated above. In the case of sterile powders for the preparation of sterile injectable solutions, the preferred methods of preparation are vacuum drying and the freeze-drying technique which yield a powder of the active ingredient plus any additional desired ingredient from previously sterile-filtered solution thereof.

When the active ingredients are suitably protected they may be orally administered, for example, with an inert diluent or with an assimilable edible carrier, or it may be enclosed in hard or soft shell gelatin capsule, or it may be compressed into tablets, or it may be incorporated directly with the food of the diet. For oral therapeutic administration, the active compound may be incorporated with excipients and used in the form of ingestible tablets, buccal tablets, troches, capsules, elixirs, suspensions, syrups, wafers, and the like. Such compositions and preparations should contain at least 1% by weight of active compound. The percentage of the compositions and preparations may, of course, be varied and may conveniently be between about 5 to about 80% of the weight of the unit. The amount of active compound in such therapeutically useful compositions in such that a suitable dosage will be obtained. Preferred compositions or preparations according to the present invention are prepared so that an oral dosage unit form contains between about 0.1 μg and 1000 μg of active compound.

The tablets, troches, pills, capsules and the like may also contain the components as listed hereafter: a binder such as gum, acacia, corn starch or gelatin; excipients such as dicalcium phosphate; a disintegrating agent such as corn starch, potato starch, alginic acid and the like; a lubricant such as magnesium stearate; and a sweetening agent such as sucrose, lactose or saccharin may be added or a flavouring agent such as peppermint, oil of wintergreen, or cherry flavouring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup or elixir may contain the active compound, sucrose as a sweetening agent, methyl and propylparabens as preservatives, a dye and flavouring such as cherry or orange flavour. Of course, any material used in preparing any dosage unit form should be pharmaceutically pure and substantially non-toxic in the amounts employed. In addition, the active compound(s) may be incorporated into sustained-release preparations and formulations.

The pharmaceutical composition may also comprise genetic molecules such as a vector capable of transfecting target cells where the vector carries a nucleic acid molecule encoding a modulatory agent. The vector may, for example, be a viral vector.

Routes of administration include, but are not limited to, respiratorally (eg. intranasally or orally via aerosol), intratracheally, nasopharyngeally, intravenously, intraperitoneally, subcutaneously, intracranially, intradermally, transdermally, intramuscularly, intraoccularly, intrathecally, intracereberally, intranasally, infusion, orally, rectally, via IV drip patch, implant and sublingual. Preferably, said route of administration is intravenously, subcutaneously, intradermally, transdermally or intranasally, more preferably, intravenously.

Yet another aspect of the present invention relates to the compositions, as hereinbefore defined, when used in any method of the present invention.

### EXAMPLES

Before the observations described herein, intercellular disulphide bonds had not been described in immunology, or, to the best of the inventors' knowledge, in biology. It was unclear whether an intercellular environment would be conducive to disulphide bond formation between proteins expressed on the surface of different cells. It was unclear whether the immunological synapse between a T cell and an antigen-presenting cell (APC) would be conducive to disulphide bond formation between a TCR on the T cell and a peptide-MHC ligand on the APC. It was unclear whether such disulphide bond formation would require structural rearrangement of the TCR or the peptide.

The consequences of increasing the affinity and/or lifetime of TCR-pMHC interactions to the supraphysiological range had produced puzzling results. Some engineered TCRs with supraphysiological affinities for, and lifetimes with, peptide-MHC ligands elicited suboptimal T cell activation. To explain those findings, it was postulated that a TCR-pMHC complex can only signal for a limited amount of time, and antigen sensitivity is optimised by intermediate-affinity TCRs that unbind and rebind pMHC repetitively, described as ‘serial binding’. As disulphide bond formation would be expected to prevent ‘serial binding’ between TCR and pMHC, it may be incompatible with T cell activation.

Consistent with a requirement for ‘serial binding’ between TCR and pMHC in T cell activation, artificial crosslinking of pre-formed TCR-pMHC complexes had been found to abolish T cell activation.

In mature T cells of healthy humans and mice, the amino acid cysteine is rarely present in the TCRα or TCRβ peptide-binding site, called complementarity-determining region 3 (CDR3). The inventor(s) hypothesised that such T cells are selectively eliminated during development in the thymus as a result of prolonged interactions with antigen-presenting cells. These long-lived interactions are a direct consequence of the cysteine in the TCR CDR3 forming a disulphide bond with a cysteine in a self-peptide presented by MHC molecules. All the data are consistent with this hypothesis.

### Example 1—Method to Predict Disulphide Bond Formation Between TCR and Peptide

TCR-peptide/MHC or TCR-peptide/HLA structures were downloaded from Protein Data Bank (www.rcsb.org) and analysed with Coot software. The TCR-peptide interface was analysed for close contact between the TCR CDR3 loops and the peptide. Close-proximity residue pairs between the TCR and the peptide were mutated to Cys in silico. Without changing the position of the β carbon atom, the rotamer (orientation) of each Cys residue was edited to allow the sulfur (S) atoms to face each other. Cys-Cys pairs in which the interatomic distance between sulfur (S) atoms was:
≥4 angstroms (Å), or the S atoms impinged on each other, were classified as unlikely to form a disulphide bond; these residues are indicated in non-bold text in Tables 3 and 4;
>3 and ≤4 Å, indicating that a disulphide bond may form with some movement of adjacent residues in the TCR and/or peptide, were classified as “low confidence”; these residues are indicated in bold and underlined text in Tables 3 and 4;
0.01-3.0 Å were classified as likely to form a disulphide bond with “high confidence”; these residues are indicated in bold (no underline) text in Tables 3 and 4;

In addition, the angle between the S atoms is important. The optimal angle formed by the 2β carbon atoms about the S-S bond should be close to 90° or −90°. If the observed angle was >30° away from these optima, then a disulphide bond is unlikely to form without structural rearrangement; these residues were represented in non-bold text in Tables 3 and 4.

Peptide residues were not mutated to Cys in silico if they were: (i) buried in the MHC cleft or (ii) a Proline. CDR3 residues were not regarded as likely to form a disulphide bond with “high confidence” if the residue to be substituted was Pro or Gly.

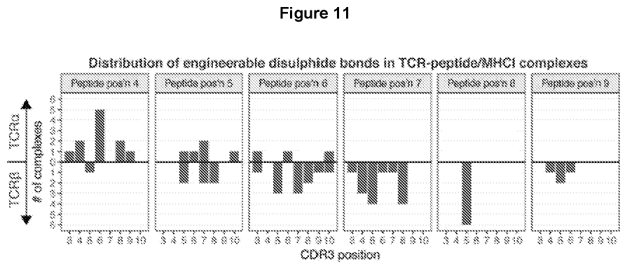

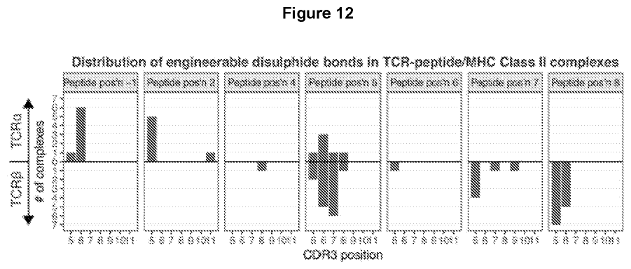

The results of these analyses are shown in Table 3 for MHC class I and Table 4 for MHC class II. A summary of the location of the residue in the CDR3 of the TCRα or TCRβ that when replaced with a cysteine would have a high likelihood of forming a disulphide bond with a corresponding or paired cysteine in a peptide bound to a MHC/HLA is shown in FIG. 11 for MHCI/HLAI and FIG. 12 for MHCII/HLAII.

### Example 2—Identifying Self-Peptide/MHC Class II Targets for TCR-Treg Therapy in Autoimmune Disease

A list of autoantigens and high-risk MHC Class II alleles in autoimmune diseases was compiled. In the example below, autoantigens in Type I Diabetes, Multiple Sclerosis (MS) and Rheumatoid Arthritis were taken from Ross K A. PLoS One 2014; 9: e101093 and Pianta A et al. J Clin Invest 2017; 127: 2946-56. Autoantigen protein sequences were obtained (https://asia.ensembl.org/Homo_sapiens).

Each possible 15-mer peptide derived from the autoantigen protein sequences was assessed for binding affinity to the relevant high-risk MHC Class II allele using NetMHCIIpan (https://services.healthtech.dtu.dk/service.php?NetMHCIIpan-3.2).

The table below shows peptides that:
a. have Cys at a potentially TCR-exposed position (P-1, P2, P3, P5, P7 or P8), wherein “P-1” means one position N-terminal to the P1 position, &
b. have a predicted binding affinity for MHCII greater than 90% of 200,000 random peptides as assessed by NetMHCIIpan.

TABLE 1
Self-peptide/MHCII targets for treatment of
autoimmune disease:

| | | | | | % rank |
|---|---|---|---|---|---|
| | | | SEQ | MHC | (binding |
| | | Peptide | ID | risk | affin- |
| Indication | Gene | (P1 to P9) | NO: | allele | ity) |
| Type I | GAD2 | LIKCDERGKM | 1 | DRB1*03: | 1.8 |
| diabetes | | | | 01 | |
| Type I | PTPRN | GCLFDRRLCS | 2 | DRB1*03: | 5.5 |
| diabetes | | | | 01 | |
| Type I | SLC30 | EYKIADPICT | 3 | DRB1*04: | 5 |
| diabetes | A8 | | | 01 | |
| Type I | AMY2A | EFRNMVTRCN | 4 | DRB1*04: | 5.5 |
| diabetes | | | | 01 | |
| Type I | PTPRN | CLLLLSSRPG | 5 | DRB1*04: | 7 |
| diabetes | | | | 01 | |
| Type I | PTPRN | EWQALCAYQA | 6 | DRB1*04: | 8 |
| diabetes | | | | 01 | |
| Type I | PTPRN | AYQAEPNTCA | 7 | DRB1*04: | 9.5 |
| diabetes | | | | 01 | |
| Multiple | MOG | ELRKFSSLCY | 8 | DRB1*15: | 2 |
| sclerosis | | | | 01 | |
| Multiple | MAG | PVLLLESHCA | 9 | DRB1*15: | 3.5 |
| sclerosis | | | | 01 | |
| Multiple | MOG | IICYNWLHRR | 10 | DRB1*15: | 4 |
| sclerosis | | | | 01 | |
| Multiple | CR1 | RFRCQPGFVM | 11 | DRB1*15: | 4.5 |
| sclerosis | | | | 01 | |
| Rheumatoid | GNS | RLMMLQSCSG | 12 | DRB1*04: | 0.9 |
| arthritis | | | | 04 | |
| Rheumatoid | GNS | CVRTMSALWN | 13 | DRB1*04: | 2.5 |
| arthritis | | | | 04 | |
| Rheumatoid | GNS | ILRSMCGYQT | 14 | DRB1*04: | 5 |
| arthritis | | | | 04 | |

### Example 3—Identifying Self-Peptide/MHC Class I Targets for TCR-T Cell Therapy in Cancer

Naturally occurring cancer associated peptides containing cysteine that are targeted by TL, were queried using TANTIGEN 2.0: Tumor T-cell Antigen Database (http://projects.met-hilab.org/tadb/). Of the 148 peptide antigens that are classified as a “Shared tumour specific antigen” and are presented by HLA Glass I molecules, 6 peptides were found to meet the criteria of (i) having cysteine at position 4, 5, 6, 7 or 8 and (ii) being verified as the antigenic target of CTL clones. These peptides are found in the table below:

TABLE 2
Cancer-testis peptide antigens containing
cysteine and targeted by CTL

| | | | | | Ref- |
|---|---|---|---|---|---|
| | | SEQ | | | erence |
| | Peptide | ID | | | (Pubmed |
| Gene | sequence | NO: | HLA | Comment | ID) |
| MAGE- | EGDCAPEEK | 15 | C*0701 | HLA | 14764691 |
| A2, | | | | expressed | |
| -A3, | | | | by 40% of | |
| -A6, | | | | Caucasians; | |
| -A12 | | | | anti-MAGE T | |
| | | | | cells can | |
| | | | | be | |
| | | | | neurotoxic | |
| MAGE-A4 | NYKRCFPVI | 16 | A*2402 | Anti-MAGE T | 16061876 |
| | | | | cells can | |
| | | | | be | |
| | | | | neurotoxic | |
| HERV- | MLAVISCAV | 17 | A2 | Target is a | 12359761 |
| KMEL | | | | largely | |
| | | | | melanoma- | |
| | | | | specific | |
| | | | | endogenous | |
| | | | | retroviral | |
| | | | | gene | |
| | | | | product | |
| CCDC110 | EYSKECLKEF | 18 | A24 | Target | 15447989 |
| (or KM- | | | | protein | |
| HN-1) | | | | provides B | |
| | | | | and T cell | |
| | | | | epitopes in | |
| | | | | cancer | |
| | | | | patients | |
| TGFBR2 | RLSSCVPVA | 19 | A2 | Peptide | 11391614 |
| | | | | formed | |
| | | | | due to | |
| | | | | frameshift | |
| | | | | mutation | |
| CCNA1 | FLDRFLSCM | 20 | A*0201 | CTL | 22529286 |
| (Cyclin- | | | | elicited | |
| A1) | | | | by peptide- | |
| | | | | pulsed DC | |

TCRs specific for these peptides can be engineered to introduce a cysteine, allowing boosting of CTL activity by forming a disulphide bond between the TCR of the CTL and the peptide-HLA complexes expressed on human cancer cells.

### Example 4—TCR and Peptide Sequences Used to Form a Model TCR-Peptide Disulphide Bond

### Materials and Methods

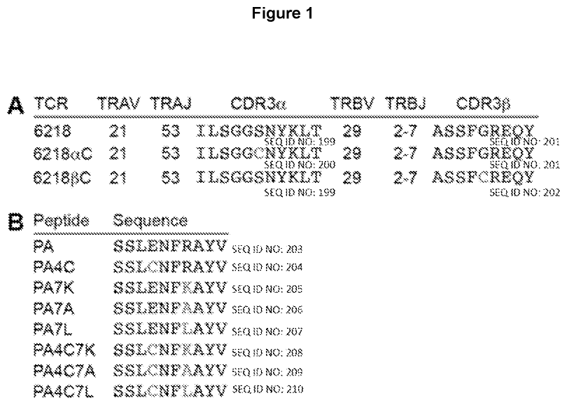

The murine 6218 TCR binds to an influenza Polymerase Acidic (PA) peptide (SSLENFRAYV) presented by H2-D^{b} (Day E. B. et al., Proc Natl Acad Sci USA, 2011; doi: 10.1073/pnas.1106851108). Based on the TCR-pMHC structure (Day E. B. et al., Proc Natl Acad Sci USA, 2011), the inventors predicted a variant of the 6218 TCR with Cys instead of Ser at the middle position (apex) of CDR3α (6218αC) would form a disulphide bond with a variant of the PA peptide with cysteine at position 4 (PA4C). The inventors also performed experiments with a variant of the 6218 TCR with Cys instead of Gly at the apex of CDR3β (6218βC) and variants of the PA and PA4C peptides in which the Arg at position 7 was substituted with Lys (K), Ala (A) or Leu (L) (FIG. 1).

### Example 5—Confirmation of Disulphide Bond Formation Using X-Ray Crystallography

### Materials and Methods

To synthesise TCR and peptide/MHC (pMHC) molecules, DNA fragments, optimised for bacterial expression, encoding the mouse variable domains of the 6218 TCR (Day E. B. et al., Proc Natl Acad Sci USA, 2011) and human constant domains were cloned into a pET30 expression vector (Genscript). In addition, the 6218αC TCR was cloned similarly with Ser 10a replaced by Cys10α. The 6218 TCRα, 6218 TCRβ, and 6218αC TCRα chains were expressed in BL21 E. coli cells separately as inclusion bodies. Functional and soluble TCRs were produced by refolding equal amounts of α- and β-chains for 3 days as described (Day E. B. et al., Proc Natl Acad Sci USA, 2011), followed by dialysis into 10 mM Tris-HCl pH 8.0. The refolded TCRs were purified by anion exchange and size-exclusion chromatography. Human P2m and H2-D^{b} heavy chain (residues 1-274) fused to a BirA-substrate peptide were expressed separately in BL21 E. coli cells and extracted as inclusion bodies. These inclusion bodies (30 mg H2-D^{b} and 10 mg of β2m) were refolded with 4 mg of either the PA peptide (SSLENFRAYV) or a variant peptide (Genscript) for 3 hours. Folded pMHC complexes were purified by anion exchange and size exclusion chromatography.

To perform X-ray crystallography, crystal screens were set up via sitting-drop, vapour diffusion at 20° C. with a protein:reservoir drop ratio of 1:1, at a concentration of 3 mg/mL in 10 mM Tris-HCl pH 8, 150 mM NaCl. Crystals of 6218-PA/H2-D^{b}, 6218-PA4C/H2-D^{b} and 6218αC-PA4C/H2-D^{b} complexes were grown in 20% (w/v) PEG3350, 0.2 M NaF, 0.05 M NaFormate and 3% (w/v) 1,5-Diaminopentane dihydrochloride. Crystals of H2-D^{b} in complex with PA4C were grown at 3 mg/mL in 20% (w/v) PEG2000, 0.1 M KSCN and 2% (w/v) 2-Methyl-2,4-pentanediol. All crystals were soaked in a cryoprotectant solution containing mother liquor solution with the PEG3350 concentration increased to 30% (w/v) and then flash-frozen in liquid nitrogen. The data were collected using AS GUI on the MX2 beamline at the Australian Synchrotron, part of ANSTO, Australia (Aragao D. et al, J Synchrotron Radiat, 2018; doi: 10.1107/S1600577518003120). The data were processed using XDS (BUILT=20161205) (Kabsch W, Acta Crystallogr D Biol Crystallogr, 2010; doi: 10.1107/S0907444909047337). Data were scaled and reduced using the Pointless and Aimless program (Evans P. R., Acta Crystallogr D Biol Crystallogr, 2011; doi: 10.1107/S090744491003982X) from the CCP4 suite (version 7.0.077). Structures were determined by molecular replacement using the PHASER program (version 2.8.3) (McCoy A. J., J Appl Crystallogr 2007; doi: 10.1107/S0021889807021206) from the CCP4 suite (1994) (version 7.0.077) with a model of H2-D^{b} without the peptide derived from PDB ID: 3PQY. Manual model building was conducted using COOT (version 0.8.9.2) (Emsley P, Acta Crystallogr D Biol Crystallogr, 2010; doi: 10.1107/S0907444910007493) followed by refinement with BUSTER (version 2.10.3) (Smart O. S. et al., Acta Crystallogr D Biol Crystallogr, 2012; doi: 10.1107/S0907444911056058). All molecular graphics representations were created using PyMOL

### Results

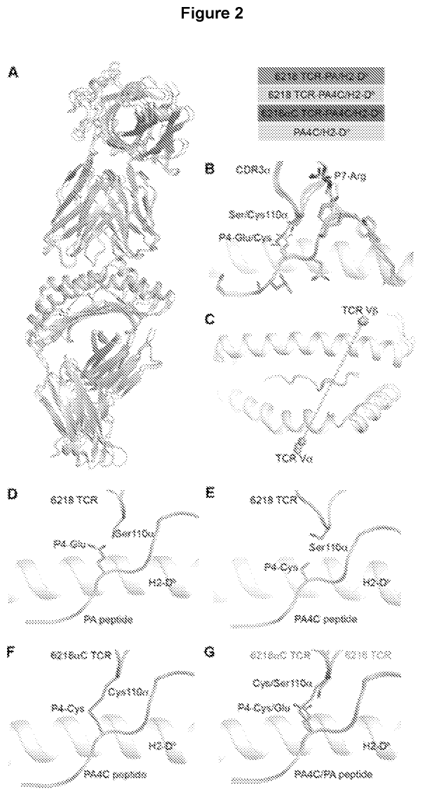

Overlay of the 6218 TCR-PA/H2-D^{b}, 6218 TCR-PA4C/H2-D^{b} and 6218αC TCR-PA4C/H2-D^{b} complexes revealed similar binding modes (FIG. 2A-C, G). The inventors observed a disulphide bond between the free Cys of the 6218αC TCR and the P4-Cys in the PA4C peptide (FIG. 2F). The disulphide bond is formed without requirement for structural rearrangement of the peptide, the CDR3, or docking topology of TCR chains (FIG. 2A-G). The P4-Cys rotamer in the disulphide bond (FIG. 2F) was distinct from the P4-Cys rotamer observed in the unbound PA4C/H2-D^{b} structure (FIG. 2B) and in the 6218 TCR-PA4C/H2-D^{b} complex (FIG. 2E), giving a potential explanation why some 6218αC TCRs bind and release PA4C/H2-D^{b} monomers in solution until disulphide bond formation occurs (see below).

### Example 6—Disulphide Bond Formation Increases T-Cell Sensitivity to Peptide Antigen

### Materials and Methods

5KC-73.8.20 (5KC) cells, which lack an endogenous TCRβ chain (White J. et al., J Exp Med, 1993; doi: 10.1084/jem.177.1.119), were sorted for loss of CD4 to establish a CD4⁻ CD8⁻ cell line and maintained in Dulbecco's modified eagle medium supplemented with 10% fetal calf serum (Gibco, Amarillo, TX, Cat. no. 10437028), 2 mM L-glutamine (Gibco, Cat. no. 25030149), 1 mM sodium pyruvate (Gibco, Cat. no. 11360070), 100 μM Non-essential amino acids (Gibco, Cat. no. 11140050), 5 mM HEPES buffer (Gibco, Cat. no. 15630080), 55 μM 2-mercaptoethanol (Gibco, Cat. no. 21985023), 100 units/mL penicillin and 100 μg/mL streptomycin (Gibco, Cat. no. 15140122) (cDMEM). 5KC cells were transduced to express mouse CD8αβ and either the 6218 or 6218αC TCR encoded in the pMIGII retroviral vector and sorted for equivalent expression of CDαβ and TCRβ. 5×10⁴ transduced 5KC cells were incubated with 10⁵ DC2.4 mouse dendritic cells (Shen Z. et al., The Journal of Immunology 158, 2723-2730 (1997)), in the absence or presence of graded concentrations of peptide or anti-CD3 (10 μg/mL pre-bound to plate overnight), for 16 hours. Supernatants were collected and assayed for IL-2 concentration using the BD OptEIA Mouse IL-2 enzyme-linked immunosorbent assay (ELISA) kit. EC₅₀ and HillSlope (h) values were determined using nonlinear regression (4-parameter dose-response curves) in GraphPad Prism.

### Results

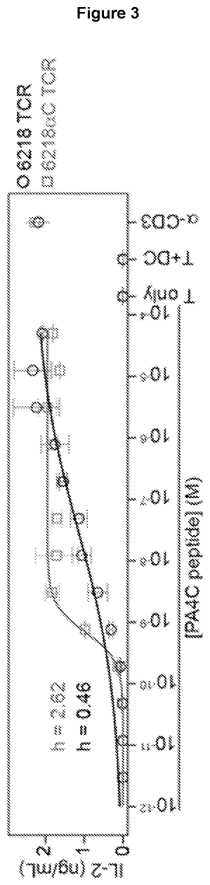

Increased T-cell sensitivity to peptide antigen was observed in cells transduced with the 6218αC TCR. While 1 nM of PA4C was needed to induce a half-maximal response (EC₅₀) in cells expressing the 6218αC TCR, it took 49 nM to induce a similar response in the 6218 TCR cells; the dose-response curve was also steeper, as demonstrated by the greater h value, for cells expressing the 6218αC TCR (FIG. 3).

### Example 7—an Assay for Disulphide Bonding Between Purified TCR and Peptide/MHC Proteins

### Materials and Methods

To perform Surface Plasmon Resonance (SPR) assays, each soluble TCR was immobilized onto a CM5 sensor chip via amine coupling. 6218αC TCRs formed disulphide-bonded homodimers, which did not bind to PA/H2-D^{b} or PA4C/H2-D^{b} unless TCR dimerisation was removed using a 10-min injection of 1 mM DTT at 10 μL/min. The DTT-treated flow cell was then equilibrated for 3 h in running buffer to reach a steady baseline. PA/H2-D^{b} or PA4C/H2-D^{b} was flowed over the TCRs in a series of 1-min injections at increasing concentrations (1.5 μM, 4.4 μM, 13.3 μM, 40 μM and 120 μM) using a 1 in 3 dilution. To demonstrate induction of dissociation for PA4C/H2-D^{b} and 6218αC TCR, samples of PA4C/H2-D^{b} were treated with 2 mM DTT overnight at 4° C. before dilution into running buffer without the presence of DTT. In assays involving longer pMHC injection times, sequential 1-min injections of a negative control pMHC (Influenza Virus NP265-274/HLA-A*03) and a positive control pMHC (PA/H2-D^{b}) preceded the PA4C/H2-D^{b} monomer injections of 5 or 20 minutes; in these assays, the concentration of all pMHC monomers was 100 μM. For assays involving pMHC injection times >1 min, to account for differences in the amount of immobilized 6218αC TCR between sensor chips, the inventors normalized the Response Units (RU) using the formula, K_{D}=[6218αC]×[PA/H2-D^{b}]/[6218αC-PA/H2-D^{b}]. As K_{D}=64.1 μM and [PA/H2-D^{b}]=100 μM, the “Normalized RU” value=1 (corresponding to an estimated 6218αC TCR occupancy of 100%) was set as 1.641× the RU detected at equilibrium when the PA/H2-D^{b} positive control was flowed over the 6218αC TCR. Experiments were conducted at 25° C. on the BIAcore T100 instrument with 10 mM Tris-HCl, pH 8, 150 mM NaCl, 0.005% surfactant P20 containing 0.1% bovine serum albumin to avoid non-specific binding. Data were exported using BIAevaluation 3.0, and data points were analysed using Prism version 9 (GraphPad Software, La Jolla California USA).

### Results

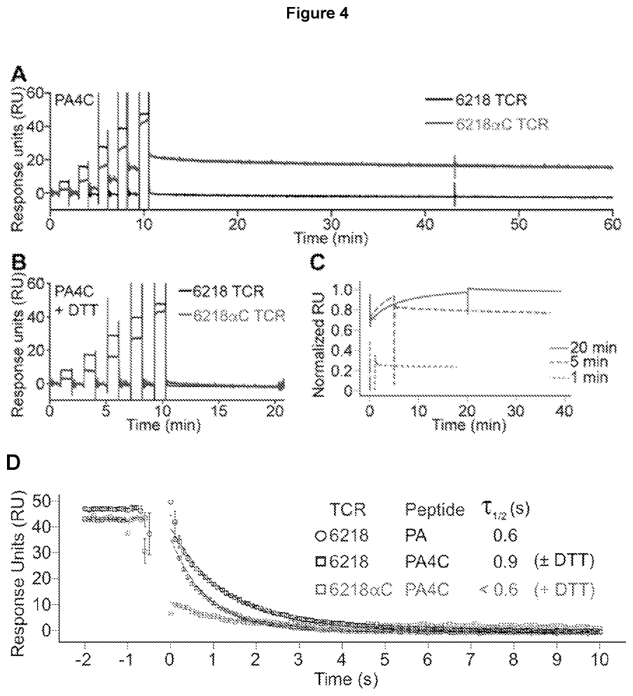

PA4C/H2-D^{b} bound similarly to the 6218 and 6218αC TCRs during the association phase, but the dissociation of pMHC and TCR was affected. The sensorgram for the 6218αC TCR did not return to baseline after injection of PA4C/H2-D^{b}, suggesting briefly exposing the 6218αC TCR to PA4C/H2-D^{b} produced distinct short-lived and long-lived subpopulations of 6218αC TCR-PA4C/H2-D^{b} complexes (FIG. 4A). Addition of the reducing agent, dithiothreitol (DTT), which inhibits disulphide bond formation, caused the 6218αC TCR sensorgram to return to baseline after PA4C/H2-D^{b} injection ended, demonstrating the long-lived subpopulation of 6218αC TCR-PA4C/H2-D^{b} complexes was dependent on disulphide bond formation (FIG. 4B). Increasing the duration of injection from 1 min to 5 min or 20 min increased the proportion of binding that persisted after PA4C/H2-D^{b} injection ended (FIG. 4C). While long-lived 6218αC TCR-PA4C/H2-Db complexes persisted for 1 h (FIG. 4A), the half-life of short-lived 6218αC TCR-PA4C/H2-D^{b} complexes was <1 s (FIG. 4D). As short-lived 6218αC TCR-PA4C/H2-D^{b} complexes turned over within seconds, whereas long-lived complexes accumulated on a timescale of minutes, most associations between the 6218αC TCR and PA4C/H2-D^{b} did not result in disulphide bond formation. These data are consistent with reversible interactions occurring between the 6218αC TCR and PA4C/H2-D^{b} until disulphide bond formation enables covalently bound 6218αC TCR-PA4C/H2-D^{b} complexes to persist.

### Example 8—an Exposed Cysteine in CDR3 Promotes Above-Threshold TCR Signaling and T-Cell Tolerance Induction in the Thymus

### Materials and Methods

DNA encoding the 6218 TCRα and TCRβ genes separated by the “cleavable” P2A peptide were synthesised (Genscript Biotech Corporation, Piscataway, NJ). Cys-encoding codons were introduced into the CDR3α (Ser110α replaced by Cys110α) or CDR33 (Gly109β replaced by Cys109p) sequence using PCR mutagenesis. DNA constructs were cloned into the pMSCV-IRES-GFP II (pMIGII) vector, which encodes GFP under the control of an internal ribosomal entry site (Holst J. et al., Nat Protoc, 2006; doi: 10.1038/nprot.2006.61). Rag1^{-/-} BM cells were retrovirally transduced in vitro as described (Holst J. et al., Nat Protoc, 2006). BM cells from C57BL/6 (B6) mice were depleted of T cells using a mouse CD38 MicroBead Kit (Miltenyi, Bergisch Gladbach, Germany, Cat. no. 130-094-973) and an autoMACS machine (Miltenyi). Rag1^{-/-} BM cells exposed to retrovirus and T cell-depleted B6 BM cells were mixed 1:1 before i.v. injection of >2×10⁶ cells per Rag1^{-/-} recipient, which had been irradiated with x-rays (two doses of 5 Gy given 4 h apart) earlier in the day. TCR-retrogenic mice were analysed 37 d after BM transfer.

For analysis of TCR-retrogenic mice, single-cell suspensions of thymus, spleen or small intestine samples were prepared. To detect CCR7+ cells, single-cell thymocyte suspensions were incubated for 60 min at 37° C. in pre-warmed FACS buffer (PBS containing 2% v/v heat-inactivated bovine serum and 0.01% m/v sodium azide) containing phycoerythrin (PE)-conjugated anti-CCR7 (BioLegend, San Diego, CA, Cat. no. 120105). Otherwise, or additionally, cells were incubated for 30 min at 4° C. in FACS buffer containing assortments of the anti-mouse antibodies: Brilliant Violet 510 anti-TCRβ (BioLegend, Cat. no. 109233), Alexa Fluor 700 anti-CD4 (BioLegend, Cat. no. 100430), APC/Fire anti-CD8a (BioLegend, Cat. no. 100766), PE/Cy7 anti-CD8β.2 (BioLegend, Ca. no. 140416), VioBlue anti-CD45 (Miltenyi, Cat. no. 130-102-430) and propidium iodide solution (BioLegend, Cat. no. 421301). Samples were then washed in FACS buffer and analysed using an LSRFortessa X-20 flow cytometer (Becton Dickinson, Franklin Lakes, NJ). Data were analysed using FlowJo software (FlowJo LLC, Ashland, Oregon).

### Results

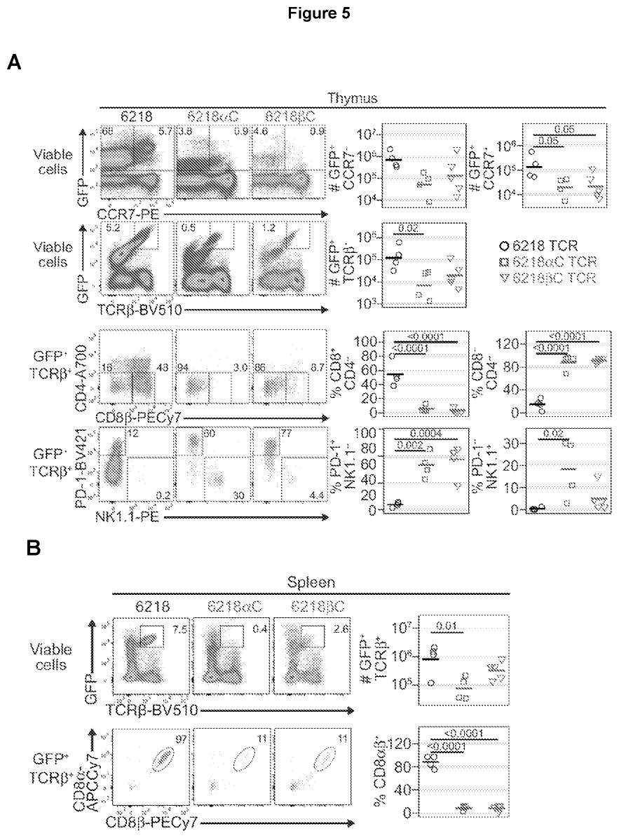

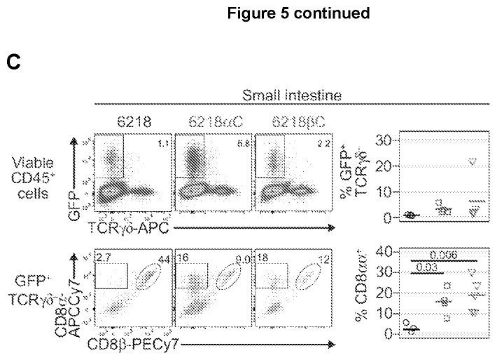

GFP⁺ thymocyte frequencies were significantly lower in the 6218αC and 6218βC groups, including at the immature CCR7⁻ stage of development (FIG. 5A). The 6218 TCR induced efficient thymic differentiation of naïve T cells, characterised by high expression of CCR7, whereas the 6218αC and 6218βC TCRs did not (FIG. 5A). In the spleen, the numbers of GFP⁺ TCRβ⁺ cells expressing CD8αβ, characteristic of conventional CD8⁺ T cells, were highest in the 6218 group (FIG. 5B). However, intestinal CD8αα intraepithelial lymphocyte (IEL) populations were larger in the 6218αC and 6218βC groups (FIG. 5C). Induction of thymocyte deletion and CD8αα IEL differentiation indicate that the 6218αC and 6218δC TCRs induced stronger TCR signaling than the 6218 TCR.

### Example 9—an Exposed Cysteine in CDR3 Affects the Abundance and Distribution of Developing T Cells in the Thymus

### Materials and Methods

Thymus samples from TCR-retrogenic mice were immersed in 50% ethanol/5% acetic acid/45% water for 10 min before transfer to 10% neutral buffered formalin overnight. Fixed samples were then exposed to a 4-hour cycle of graded ethanols and xylene using a Peloris II Tissue Processor (Leica, Wetzlar, Germany) before embedding in Paraplast wax (P3558, Sigma-Aldrich, St. Louis, MO) and sectioning onto superfrost plus slides at a thickness of 4 μm using a RM2235 microtome (Leica). Primary antibodies, Chicken anti-GFP (ab13970, Abcam, Cambridge, UK) and Rabbit anti-Cytokeratin 14 (K14) (ab197893, Abcam), were used at a dilution of 1 in 200; secondary antibodies, Goat Anti-Chicken IgY A647 (ab150175, Abcam) and Donkey Anti-Rabbit IgG A488 (711-545-152, Jackson ImmunoResearch, West Grove, PA), at 1 in 500. Staining was performed using an Autostainer Link 48 (Dako, Glostrup, Denmark) with the following incubations, each interspersed by one or two 5-minute incubations in Wash Buffer (K8007, Agilent, Santa Clara, CA): Target Retrieval Solution S1699 (Agilent) at 98° C. for 30 minutes, Protein Block X0909 (Agilent) at room temperature (RT) for 60 minutes, primary antibodies at RT for 60 minutes, secondary antibodies at RT for 60 minutes, DAPI at RT for 15 minutes. Imaging was performed on an Olympus VS120 virtual slide microscope with a UPLS APO 20× lens with 0.75 NA and captured with an Olympus XM10 digital camera. A DAPI/FITC/CY5 filter set was used with the same exposure settings for all sections. GFP+ cell density was determined using Fiji (J. Schindelin J. et al., Nat Methods, 2012; doi: 10.1038/nmeth.2019) with cortex defined as K14⁻ areas and medulla as K14⁺ areas. Images were generated using cellSens Dimension version 4.1 software (Olympus Corporation, Tokyo, Japan).

### Results

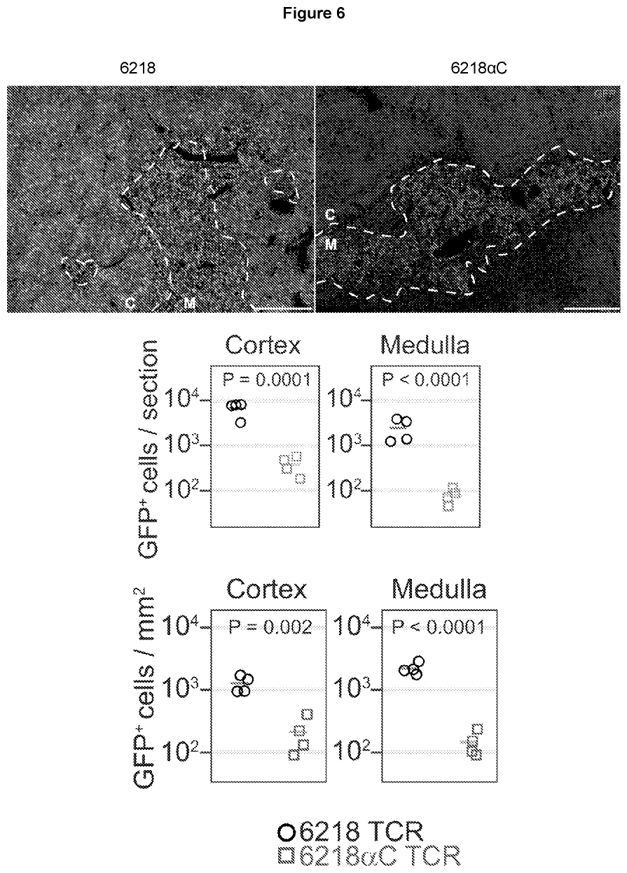

GFP⁺ thymocytes expressing the 6218 TCR were in the cortex and medulla of the thymus, reflecting the expected migration from cortex to medulla during naïve T cell differentiation (FIG. 6). In comparison, fewer GFP⁺ thymocytes expressing 6218αC were detected in the cortical and medullary regions of thymus sections, consistent with aborted, altered or arrested development (FIG. 6).

### Example 10—Substitutions at P7 of the Peptide Affect the Probability of TCR Binding to pMHC Tetramers Regardless of Cysteine Residues

### Materials and Methods

To make TCR transfectants, 293T (human embryonic kidney cell line) cells maintained in cDMEM were mixed with FuGENE 6 Transfection Reagent (Promega Corporation, Cat. no. E2691) and two pMIG II plasmids encoding GFP. One plasmid contained DNA sequences encoding mouse CD3γ, CD3β, CD3ε and CD3ζ separated by the 2A peptide. Another plasmid contained DNA sequences encoding the TCRα and TCRβ chains of the 6218 or 6218αC TCR separated by DNA encoding the 2A peptide. 48 h after transfection, TCR transfectants were incubated with PE-conjugated pMHC tetramers for 1 h at RT. Cells were washed, incubated with APC anti-TCRβ (BioLegend, Cat. no. 109212) and LIVE/DEAD Fixable Aqua Dead Cell Stain (Thermofisher, Waltham, MA, Cat. no. L34957) for 30 min, then washed before flow cytometry analysis. To make pMHC tetramers, pMHC molecules prepared with peptide variants as described above were biotinylated using BirA biotin ligase with the addition of D-biotin (Astral Scientific, Sydney, Australia, Cat no. BIOBB0078), and tetramerised by addition of PE-Streptavidin (BioLegend, Cat. no. 405204) at a 4:1 molar ratio. DNA encoding BirA biotin ligase was cloned into a pcDNA3.1 expression vector with a His-tag (Genscript Biotech), and the protein was expressed in BL21 E. coli cells, then purified using Ni-NTA agarose beads (Machery-Nagel, Duren, Germany, Cat. no. 745400.100).

### Results

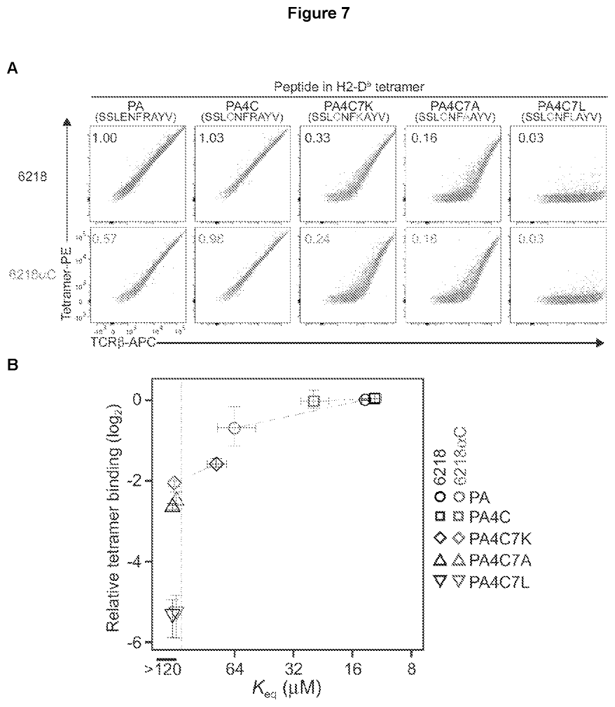

In cell-based pMHC tetramer binding assays, P7 substitutions in the PA4C peptide resulted in a gradation of binding levels, including an absence of detectable binding; however, at each step in the gradation the inventors obtained similar results for the 6218 and 6218αC TCRs (FIG. 7).

### Example 11—Substitutions at P7 of the Peptide Affect the Probability of Binding Between Purified TCR and pMHC Proteins Regardless of Cysteine Residues

### Materials and Methods

SPR assays were performed as described above using a series of 1-min injections with increasing pMHC concentrations (maximum concentration of 120 μM or, in the cases of PA4C7L/H2-D^{b} and PA7L/H2-D^{b}, maximum concentration 240 μM).

### Results

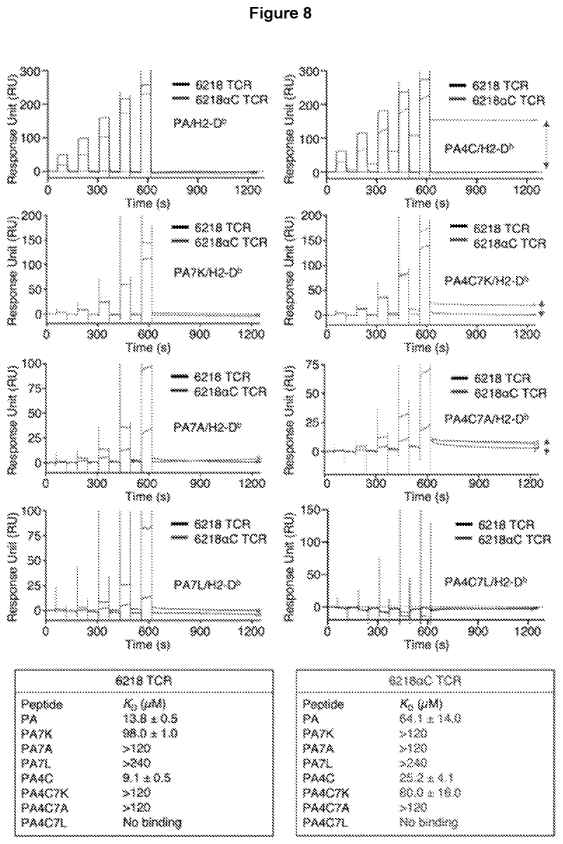

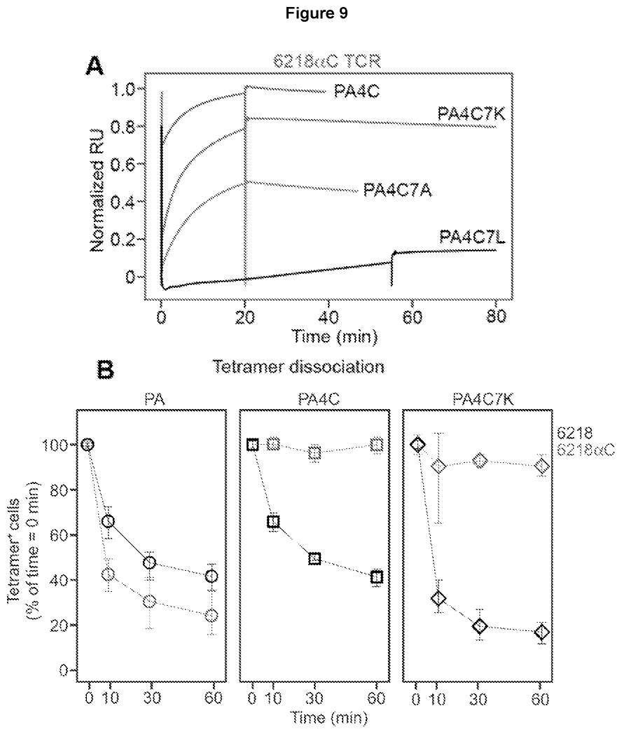

SPR results revealed that each P7 substitution decreased binding to both 6218 and 6218αC TCRs (FIG. 8).

### Example 12—Disulphide Bond Formation Prevents Dissociation of TCR from Peptide/MHC

### Materials and Methods

Immobilised TCRs were prepared for SPR assays as described above. The inventors performed sequential 1-min injections of a negative control pMHC (Influenza Virus NP265-274/HLA-A*03) and a positive control pMHC (PA/H2-D^{b}) prior to the test pMHC monomer [PA4C/H2-D^{b}, PA4C7K/H2-D^{b}, PA4C7A/H2-D^{b}, or PA4C7L/H2-D^{b}] injections of 20 or 50 minutes, followed by injection of buffer. The concentration of all pMHC monomers was 100 μM. SPR data were analysed as described above and presented as “Normalized RU”. For tetramer dissociation assays, TCR transfectants were stained with 5 μg/mL pMHC tetramers for 1 h at RT, then washed and incubated for 10, 30, or 60 min with 25 μg/mL anti-H2-D^{b}/K^{b} (BD Biosciences, clone 28-8-6, Cat. no. 553575) to prevent tetramer rebinding, then washed and stained with anti-TCRβ-APC and LIVE/DEAD Stain before flow cytometric analysis. Graphs show the tetramer⁺ cell frequency as a percentage of the corresponding sample without anti-H2-D^{b}/K^{b} (time=0 min). Symbols show the mean and error bars the range of 4 samples per condition compiled from 2 experiments.

### Results

Extended SPR injection times revealed persistent interactions between all P4-Cys-containing pMHC monomers and the 6218αC TCR (FIG. 9A). Furthermore, P4-Cys-containing pMHC tetramers did not dissociate from cells expressing the 6218αC TCR (FIG. 9B). Thus, although P7 substitutions reduced the probability of TCR binding to pMHC in SPR and tetramer assays (FIGS. 7 and 8), persistent interactions still occurred between all P4-Cys-containing pMHC molecules and the 6218αC TCR, consistent with disulphide bond formation (FIG. 9).

### Example 13—Disulphide Bond Formation Reveals a Trade-Off Between T-Cell Sensitivity to, and Discrimination of, Peptide Antigens

### Materials and Methods

T-cell stimulation assays using 5KC T cells and DC2.4 mouse dendritic cells were performed as described above using the PA4C, PA4C7K, PA4C7A, PA, PA7K and PA4C7L peptides.

### Results

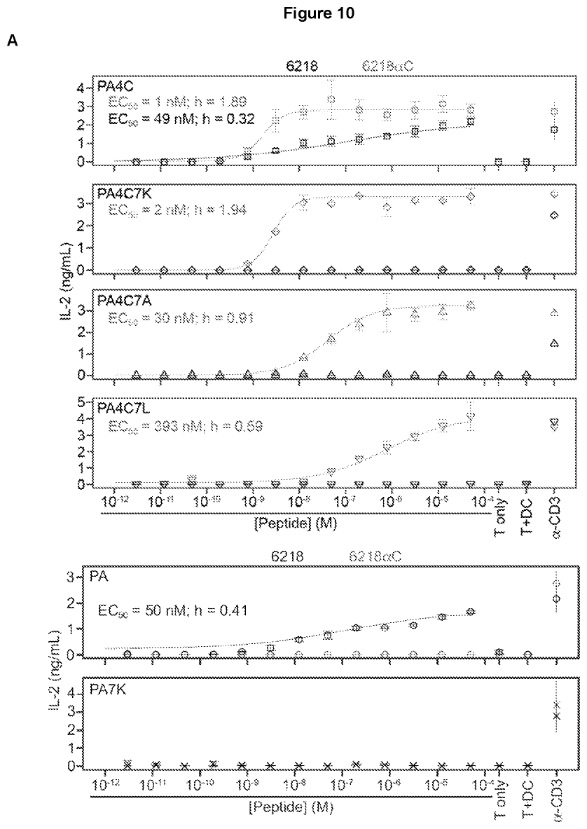

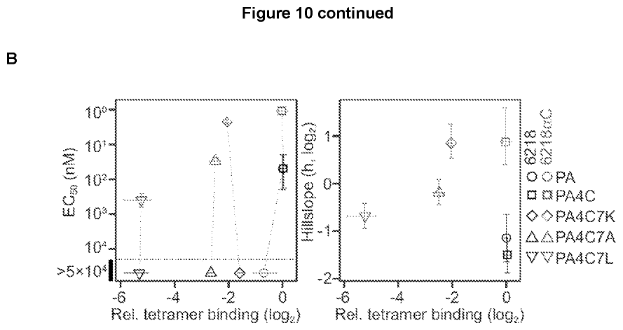

Cells expressing the 6218 TCR were not activated by any peptide with a P7 substitution, whereas cells expressing the 6218αC TCR were activated by all peptides with a P4-Cys (FIG. 10A). Thus, the lower T-cell sensitivity in noncovalent compared to covalent antigen recognition (FIG. 3) is coupled with superior discrimination of peptide antigens (FIG. 10A). However, a level of peptide discrimination was retained in covalent antigen recognition as the EC₅₀ and steepness of dose-response curves (h) for P4-Cys-containing peptides corresponded to the hierarchy of their probabilities of binding to the 6218αC TCR in tetramer and SPR assays (FIGS. 7 and 8).

Thus, by introducing Cys residues into a model TCR-pMHC combination and making additional substitutions at P7 of the peptide, the inventors examined interactions in which the observed binding between TCR and pMHC varied from the physiological anti-viral range to below the limit of detection in tetramer and SPR assays. Cys residues did enable persistent binding between TCR and pMHC, consistent with the formation of disulphide bonds that prevented the dissociation of covalently bound TCR-pMHC complexes. However, at each step in the gradation of binding, Cys residues had little effect on fluorescence in cell-based tetramer assays or K_{D} values in SPR assays (FIG. 10B).

Based on the similar probability of TCR binding to pMHC, ˜1 nM of PA4C peptide in the coculture assay would be expected to induce similar rates of TCR-pMHC complex formation in cells expressing the 6218 TCR or the 6218αC TCR. The capacity of ˜1 nM of PA4C peptide to activate cells expressing the 6218αC TCR, but not cells expressing the 6218 TCR, is probably not due to a difference in TCR-pMHC complex formation rate but can be explained by covalently bound 6218αC TCR-PA4C/H2-D^{b} complexes persisting long enough to activate the TCR signaling cascade. However, cells expressing the 6218αC TCR required >2 nM of the PA4C7A or PA4C7L peptides to become activated. This is attributable to the probability of binding to the 6218αC TCR being lower for PA4C7A/H2-D^{b} and PA4C7L/H2-D^{b} compared to PA4C/H2-D^{b}. At low pMHC density, the lifetime of TCR-pMHC interactions was a limiting factor during noncovalent antigen recognition whereas the probability of TCR binding to pMHC was a limiting factor during covalent antigen recognition.

### Example 14—Role of Zap70 and MHC in Cysteine-Linked T Cell Fate Skewing

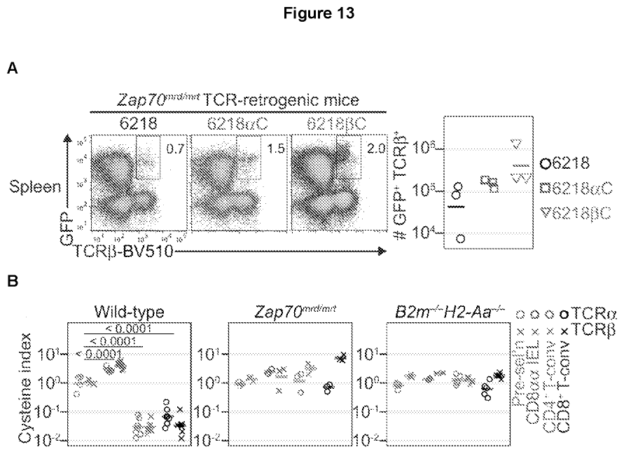

In mice with attenuated function of the TCR signaling protein, Zap70, thymocytes that would normally be deleted undergo aberrant development into CD4⁺ or CD8αβ⁺ T-conv cells. To explain that finding, TCR-pMHC interactions that should induce strong TCR signaling are thought to induce only weak TCR signaling due to the attenuated signal transmission through mutant Zap70. To test whether a Cys-containing CDR3 normally elicits strong TCR signaling in vivo, the inventors looked for evidence that thymocytes with a Cys-containing CDR3 undergo aberrant development into T-conv cells in Zap70^{mrd/mrt} mice. The inventors analysed TCR-retrogenic mice bearing Zap70^{mrd/mrt} BM cells transduced with the 6218, 6218αC or 6218βC TCR. The numbers of GFP⁺ TCRβ⁺ cells in the spleen were similar, albeit relatively low, when these TCRs were expressed in Zap70^{mrd/mrt} cells (FIG. 13A). These data support the hypothesis that a Cys-containing CDR3 normally elicits strong TCR signaling in vivo.

Although the TCR-retrogenic experiments above provide functional evidence that a Cys in CDR3 skews T cell fate, those experiments were limited to monoclonal T cell populations. To generalize those findings, the inventors sequenced the TCR repertoires of polyclonal T cell populations, including in mice with genetic defects in TCR-pMHC signaling.

### Materials and Methods

Whole thymus or spleen suspensions were prepared by pushing organs through a 70 μm sieve in sort buffer (PBS containing 2% v/v heat-inactivated fetal calf serum and 2 mM EDTA). Small intestine was first cut longitudinally and then into pieces ˜0.5 cm long while being kept moist with washing medium (WM, DMEM containing 2.5% v/v heat-inactivated bovine serum and 10 mM HEPES) and placed in a 50 mL tube containing ˜15 mL ice-cold WM. Intestinal contents were removed by cycles of vortexing for 5 s, then removing the supernatant by using a strainer to retain intestinal tissue and resuspending in 15 mL WM, until supernatant was clear.

Tissue pieces were then incubated for 15 minutes at 37° C. with gentle rotation in dissociation buffer (calcium- and magnesium-free PBS containing 5% v/v heat-inactivated bovine serum plus 2 mM EDTA). After vortexing for 15 s, tissue pieces were removed using a strainer and discarded, while the supernatant was passed through a 70 μm sieve, pelleted by centrifugation, resuspended in 5 mL of 40% Percoll and overlaid onto 5 mL 80% Percoll in a 15 mL tube.

After centrifugation for 20 min at 900 g at 20° C., material at the interface was collected, transferred to a fresh 15 mL tube containing 10 mL of sort buffer, pelleted and incubated with fluorescently conjugated antibodies for FACS analysis.

For CCR7 staining of thymocytes, suspensions were incubated for 60 min at 37° C. in 1 mL pre-warmed sort buffer containing phycoerythrin (PE)-conjugated anti-CCR7 (BioLegend, San Diego, CA, Cat. no. 120105). For staining of other cell surface markers, each thymus or spleen sample was incubated in 1 mL sort buffer, and each small intestinal sample was incubated in 0.5 mL sort buffer, containing fluorescently conjugated antibodies for 30 min at 4° C.

After washing, cells were passed through a 40 μm sieve before using an Influx Cell Sorter instrument (Becton Dickinson, Franklin Lakes, New Jersey) to sort T cell subsets (typically 5×104 cells per sample) into 1.5 mL Eppendorf tubes containing 350 QL Buffer RLT from the RNeasy Mini Kit (Qiagen, Hilden, Germany, Cat. no. 74106). Samples were then frozen by pushing into dry ice and stored at −80° C. until RNA isolation.

RNA was isolated using the RNeasy Mini kit with an elution volume of 22 μL, from which 12 μL were used to synthesise cDNA with the QuantiTect Reverse Transcription Kit (Qiagen, Cat. no. 205311). Using 5 μL of cDNA per reaction, TCRβ transcripts were PCR amplified using a Q5@High-Fidelity PCR Kit (New England BioLabs, Ipswich, MA, Cat. no. E0555L) and a mix of 19 Trbv-specific forward primers and a single Trbc-specific reverse primer and TCRα transcripts were PCR amplified using a mix of 23 or 24 Trav-specific forward primers and a single Trac-specific reverse primer (purchased from GeneWorks, Adelaide, Australia). Forward and reverse primers had distinct 5′ overhang adapter sequences that enabled addition of sample-specific indices and P5/P7 sequencing adapters in a second PCR using the Nextera XT DNA Library Preparation Kit (Illumina, San Diego, CA, Cat. no. FC-131-1096). Before the second PCR, AMPure XP magnetic beads (Beckman Coulter, Brea, CA, Cat. no. A63881) were used to enrich amplicons >100 bp. Conditions for the first PCR were 98° C. for 5 min, 20 cycles of 98° C. for 10 s, 60° C. for 30 s and 72° C. for 30 s, followed by 72° C. for 2 min. Conditions for the second PCR were 72° C. for 3 min and 98° C. for 30 s, 20 cycles of 98° C. for 10 s, 63° C. for 30 s and 72° C. for 30 s, followed by 72° C. for 1 min.

After determining amplicon concentrations using a QIAxcel capillary electrophoresis machine (Qiagen), equimolar amounts of amplicons from up to 270 samples were pooled into a single tube, concentrated using AMPure XP magnetic beads and then 300-500 bp amplicons were gel-purified before sequencing on a NextSeq machine (Illumina), with a short read 1 of 6 bases followed by a read 2 of 145 bases.

Sequences were aligned to mouse TCR genes using molecular identifier groups-based error correction (MIGEC) software (version 1.2.6). Subsequent analyses were performed using RStudio software (version 2022.02.3 Build 492). Sequences with a CDR3 that was out-of-frame or contained a stop codon were excluded. A clone was defined as a unique combination of Trav or Trbv gene and CDR3 nucleotide sequence. Each clone was counted only once per sample regardless of its number of reads.

CDR3 length was determined using the CDR3-IMGT definition, which excludes the conserved N-terminal Cys and C-terminal Trp or Phe from the CDR3. For a CDR3 sequence of n amino acids, the amino acid at the largest position not greater than (n/2+1) was defined as the middle CDR3 position (apex). The cysteine index for each sample equals the percentage of clones with Cys within 2 positions of the CDR3 apex. Sequences detected only once or twice in any given sample were excluded from cysteine index calculations. For the sample that had 0 clonotypes with Cys within two positions of the CDR3 apex, the cysteine index was defined as the reciprocal of the number of clones in the sample, expressed as a percentage. As Trbv1 sequences can have a germline-encoded Cys at CDR3 position 2, which is within 2 positions of the apex of CDR3 sequences <8 amino acids long, the inventors excluded Trbv1 sequences with a CDR3 length <8 amino acids.

### Results

In wild-type mice (Left panel, FIG. 13B), Cys-containing CDR3 were enriched in CD8αα IEL and depleted in CD4+ and CD8+T-conv cells compared to pre-selection thymocytes. However, pre-selection thymocytes and mature T cell subsets in Zap70^{mrd/mrt} mice had similar frequencies of Cys-containing CDR3 (Middle panel, FIG. 13B), indicating that polyclonal thymocytes with a Cys-containing CDR3 undergo aberrant development into T-conv cells in Zap70^{mrd/mrt} mice. To test whether the effect of a Cys-containing CDR3 depends on pMHC ligands, the inventors sequenced the TCR repertoires of B2m^{-/-}H2-Aa^{-/-} mice, which lack cell-surface expression of MHC proteins. The inventors found similar frequencies of Cys-containing CDR3 in pre-selection thymocytes and mature T cells from B2m^{-/-}H2-Aa^{-/-} mice (Right panel, FIG. 13B). Collectively, these results demonstrate the differential expression of Cys in mature TCR repertoires is a direct consequence of Cys-containing CDR3 inducing strong TCR signaling in response to pMHC ligands in the thymus.

### Example 15—Context-Dependent Effects of Cys on TCR-pMHC Binding

### Materials and Methods

293T TCR transfectants expressing CD3 and the 6218, 6218αC or 6218βC TCR, prepared as described above, were incubated with tetramers of H2-D^{b} loaded with PA or PA4C, and analysed by FACS.

### Results

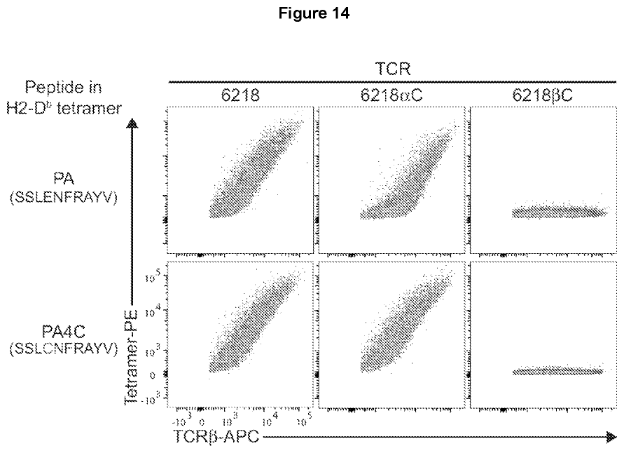

PA/H2-D^{b} and PA4C/H2-D^{b} tetramers bound to cells expressing the 6218 or 6218αC TCRs, but not to cells expressing the 6218βC TCR (FIG. 14). The absence of binding to the 6218βC TCR was expected because the Gly to Cys substitution introduces a larger side-chain, which would likely alter the conformation of the CDR3β loop and prevent close interaction between its main chain and the Arg at P7 of the peptide. To assess effects of these Cys substitutions on the TCR-pMHC interface, the inventors applied X-ray crystallography (see Example 5, FIG. 2).

### Example 16—TCRs Engineered to Form a Disulphide Bond with α3 Chain of Type IV Collagen (α3) Goodpasture's Disease

Arguably the best understood human autoimmune disease is Goodpasture's disease, which is associated with the MHC Class II (MHCII) allele, HLA-DRB1*15:01 (DR15). DR15⁺ humans and mice develop Goodpasture disease due to pro-inflammatory T cell and B cell responses towards the α3 chain of type IV collagen (α3), a component of basement membranes in kidney and lung. However, co-expression of the MHCII allele, HLA-DRB1*01:01 (DR1), induces the formation of α3/DR1-specific T-reg cells that prevent Goodpasture disease.

Remarkably, DR15 and DR1 confer susceptibility and resistance to this disease, respectively, by presenting the same peptide to CD4⁺ T cells. However, the peptide anchor residues are offset by one position so that the TCR “sees” different amino acids of the peptide when it is presented by DR15 versus DR1 (Ooi, J. D. et al. Dominant protection from HLA-linked autoimmunity by antigen-specific regulatory T cells. Nature. 545, 243-247 (2017). doi: 10.1038/nature22329).

Generation of a CD4+ T Cell Hybridoma that Binds to the α3/DR15 Antigen

To study Goodpasture disease pathogenesis, the inventors generated a CD4+ T cell hybridoma that binds to the α3/DR15 antigen.

To generate the T cell hybridoma, two female DR15-transgenic Fcgr2b^{-/-} mice aged 80 days were each immunised subcutaneously with 100 μg α3 peptide (KKDWVSLWKGFSFKK; SEQ ID NO:211) emulsified in complete Freund's adjuvant in a total volume of 100 μL, then re-immunised with 100 μg of α3 peptide emulsified in incomplete Freund's adjuvant in a total volume of 100 βL seven and 14 days later (total of three immunisations). The lysine residues at the N- and C-termini make the α3 peptide more soluble in aqueous solutions.

The mice were killed 34 days after the first immunisation and CD4+ memory T cells were isolated from pooled spleen and lymph node cells using an EasySep Mouse Memory CD4+ T Cell Isolation Kit (StemCell Technologies, Cat. No. 19767). 2×10⁵ CD4+ memory T cells were cultured in 500 μL culture medium in a single well of a 24-well plate with 2×10⁵ Dynabeads Mouse T-Activator CD3/CD28 for T-Cell Expansion and Activation (Thermofisher Scientific, Cat. No. 11453D) in the presence of 30 U/mL human IL-2 (Genscript, Cat. No. Z00368-1).

After 48 hours of culture, 2.2×10⁵ CD4+ T cells were fused with 10⁷ BWZ.36 cells (Sanderson and Shastri, Int Immunol. 1994 March; 6(3):369-76), then cell aliquots were cultured in 100 μL hybridoma medium (cDMEM+400 μg/mL hygromycin to maintain the NFAT-lacZ transgene) per well in 96-well plates following the protocol of White et al, with the addition of 100 μL of hybridoma medium containing 2×HAT Supplement (ThermoFisher Scientific, Cat. No. 21060017) on day 2 after the fusion (White et al., Methods Mol Biol. 2000; 134:185-93).

On day 10 after the fusion, half of the volume from all wells in each 96-well plate were pooled and incubated with APC-conjugated HLA-DR15 tetramers loaded with the α3 peptide (α3/DR15-APC, NIH Tetramer Core Facility, Emory University, Altanta), identifying one plate with an elevated frequency (0.2%) of α3/DR15-APC+ cells.

On day 14 after the fusion, cells pooled from all 96 wells of the plate of interest (identified on day 10) were incubated with α3/DR15-APC tetramers and 420 α3/DR15-APC+ cells were FACS sorted into 100 μL hybridoma medium containing 1×HAT Supplement in a single well of a 96-well plate using an Influx Cell Sorter instrument (Becton Dickinson, Franklin Lakes, New Jersey).

On day 27 after the fusion, the same protocol was used to sort 750 α3/DR15-APC+ cells into 100 μL hybridoma medium containing 1×HT Supplement (ThermoFisher Scientific, Cat. No. 41065012).

On day 40 after the fusion, the same protocol was used to sort individual α3/DR15-APC+ cells into 200 μL hybridoma medium in separate wells of a 96-well plate.

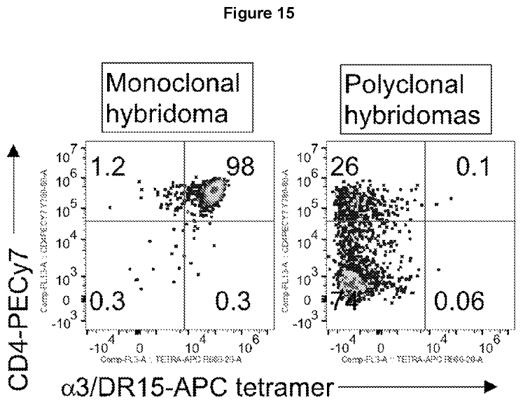

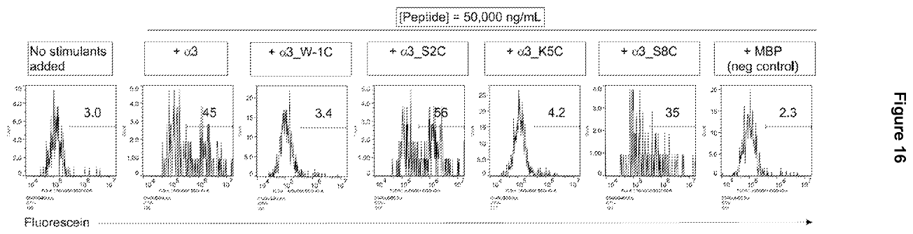

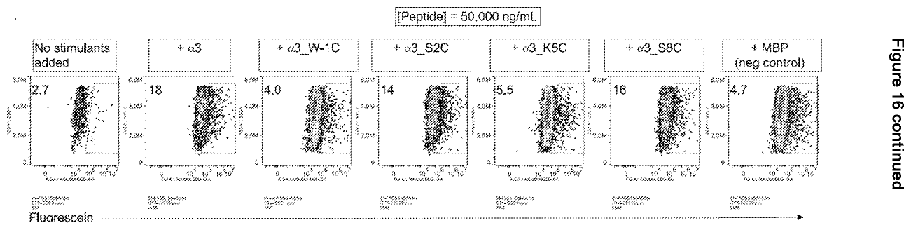

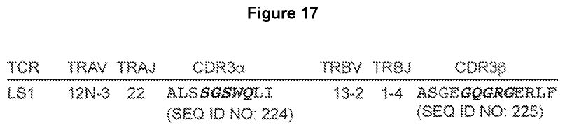

On day 50 after the fusion, FACS analysis of expanded clones demonstrated >98% of cells were α3/DR15-APC+ and CD4+(FIG. 15).

T Cell Hybridoma Activation Assays to Identify Reactivity to Variants of the α3 Peptide with Cysteine Substitutions at TCR-Exposed Positions

This hybridoma, denoted LS1, has an inducible NFAT-lacZ β-galactosidase transgene derived from the BWZ.36 cells (Sanderson and Shastri, Int Immunol. 1994 March; 6(3):369-76). Peptide-dependent TCR engagement induces transcription from the NFAT promoter and translation of lacZ β-galactosidase (lacZ) in activated cells, in which lacZ converts its substrate, fluorodeoxyglucose (FDG), to fluorescein. To provide DR15+ antigen-presenting cells, splenocytes from a naïve DR15-transgenic. Fcgr2b^{-/-} mouse were labelled with CellTrace Violet (CTV, ThermoFisher Scientific, Cat. No. C34557). CTV+DR15+ antigen-presenting cells were cultured with LS1 cells in 200 μL cDMEM in the absence or presence of one of various peptides (50 μg/mL). After 16 hours, cells were osmotically loaded with FDG (Nolan et al., Proc Natl Acad Sci USA. 1988 April; 85(8):2603-7) and analysed by FACS. Fluorescein was detectable in CTV⁻ (LS1) cells that had been cultured with a3 peptide, but not in similar cells cultured without a3 peptide (FIG. 16).

As the inventors will engineer a disulphide bond into the LS1-α3/DR15 complex, the inventors tested whether the LS1 TCR reacted to variants of the α3 peptide with cysteine substitutions at TCR-exposed positions (Ooi, J. D. et al. Nature. 545, 243-247 (2017)) (FIG. 16). LS1 cells were activated by a variant peptide with a cysteine substitution for the serine at position (P) 2 (α3_S2C; KKDWVCLWKGFSFKK; SEQ ID NO:212) or the serine at P8 (α3_S8C; KKDWVSLWKGFCFKK; SEQ ID NO:213). LS1 cells were not activated by variants of the α3 peptide with a cysteine substitution for the tryptophan at P-1 (α3_W-1C; KKDCVSLWKGFSFKK; SEQ ID NO:214) or the lysine at P5 (α3_K5C; KKDWVSLWCGFSFKK; SEQ ID NO:215), nor is it activated by a negative control peptide from myelin basic protein (MBP) (KKENPVVHFFKNIVTPKK; SEQ ID NO: 216). The TCR expressed by LS1 cells thus facilitates T cell activation by α3/DR15 and some, but not all, ligands that are closely related to α3/DR15. In other words, these data suggest the LS1 TCR binds to α3/DR15 with demonstrable specificity, like many anti-microbial or autoimmune TCR-pMHC interactions characterised previously.

### Sequencing the TCRα and TCRβ Chains Expressed by LS1 Cells to Identify Residue Candidates for Cysteine Substitution

To sequence TCR chains expressed by the LS1 hybridoma, RNA isolated from LS1 cells was reverse transcribed using Template Switching RT Enzyme Mix (New England BioLabs, Cat. No. M0466L). The reverse transcription primer was CGTCTGAACTGGGGTAGGTG (SEQ ID NO: 217) for TCRα and CTGAAAGCCCATGGAACTGC (SEQ ID NO: 218) for TCRβ. The template switch DNA-RNA oligonucleotide primer was GAATTCACCTATCAACGCAGAGTACATXXX (Where X represents riboguanosine (rG), SEQ ID NO: 219). cDNA was used as template in PCR with a forward primer AATTGAATTCACCTATCAACGCAGAG (SEQ ID NO: 220) and a reverse primer AATTCTCGAGAAGTCGGTGAACAGGCAGAG (SEQ ID NO: 221) for TCRα or a reverse primer AATTCTCGAGTGGACCTCCTTGCCATTCAC (SEQ ID NO: 222) for TCRβ. PCR products were digested with EcoRI and XhoI and then ligated into pMSCV-IRES-mCherry FP plasmid (Addgene, Cat. No. 52114), which was used to transform 10-β Competent E. coli (New England BioLabs, Cat. No. C3019H). Miniprep DNA isolated and purified from subsequent E. coli cultures was subjected to Sanger sequencing using the primer CCTCACATTGCCAAAAGACG (SEQ ID NO: 223).

Sequences were aligned to mouse TCR nucleotide sequences using IMGT/V-Quest (https://www.imgt.org/IMGT_vquest/input). Details of variable (TRAV/TRBV) and junctional (TRAJ/TRBJ) gene segments and CDR3 amino acid sequences of TCRα and TCRβ chains expressed by the LS1 T cell hybridoma (LS1 TCR) are provided in FIG. 17.

That the α3_W-1C peptide fails to activate LS1 cells suggests the LS1 TCR contacts the tryptophan at P-1 of the α3 peptide. The LS1 TCR may not dock centrally on the α3/DR15 ligand, but instead may dock further towards the N-terminal portion of the α3 peptide. The Ob.1A12 TCR, which is specific for MBP/DR15 and derives from a patient with multiple sclerosis (Wucherpfennig et al., J Exp Med. 1994 Jan. 1,179(1):279-90. doi: 10.1084/jem.179.1.279), has an unconventional docking topology that is shifted towards the N-terminal portion of the peptide (Hahn et al. Unconventional topology of self peptide-major histocompatibility complex binding by a human autoimmune T cell receptor. Nat Immunol. 6, 490-496 (2005). doi: 10.1038/ni1187). The centre of the Ob.1A12 TCR is positioned over P2 of the peptide, rather than P5 of the peptide, as observed frequently in other TCR-pMHC complexes (Hahn et al. 2005).

The Ob.1A12 TCR has a shorter CDR3α (12 aa) than CDR3β (14 aa). Interestingly, the inventors found that in LS1, CDR3α (10 aa) is also shorter than CDR3β (13 aa). This asymmetry may predispose the LS1 TCR towards a tilted docking topology on the α3/DR15 ligand, towards the DR15 β helix, as observed in the Ob.1A12-MBP/DR15 complex (Hahn et al. 2005). The LS1 TCR reacts to versions of the α3 peptide with either serine or cysteine at P2. The α3_S2C variant would appear to be a good candidate to enable S-S bond formation with variants of the LS1 TCR with a cysteine substitution in either CDR3α or CDR3β.

Engineering a Disulphide Bond into the LS1-α3/DR15 Complex

The inventors will make ten variants of the LS1 TCR with a cysteine substitution at each of the CDR3 positions shown in bold italics: ALSSGSWQLI (SEQ ID NO: 224) for CDR3α and ASGEGQGRGERLF (SEQ ID NO: 225) for CDR3β (FIG. 17). The inventors will make T cell lines expressing LS1, or one of its ten variants described above, and test the reactivity of these T cell lines to DR15+ antigen-presenting cells incubated with α3, α3W-1C, α3S2C, α3K5C, α3S8C, or a variant with cysteine instead of the leucine at position 3 (α3L3C, KKDWVSCWKGFSFKK, SEQ ID NO: 226). Disulphide bond formation is expected to increase T cell sensitivity to peptide antigen, demonstrated by T cell activation in response to lower concentrations of peptide antigen in vitro.

To confirm disulphide bond formation, each TCR of interest, together with mouse CD3, will be transfected into 293T cells to achieve cell surface TCR expression. The inventors will obtain DR15 tetramers containing a peptide of interest (for example, α3W-1C, α3S2C, α3K5C, α3S8C, α3L3C). The inventors will use a tetramer dissociation assay (as described herein) to screen each TCR/peptide combination in the presence of the anti-MHCII antibody, clone L243 (Lampson and Levy, J Immunol. 1980 July; 125(1):293-9). A lack of tetramer dissociation would provide evidence of a disulphide bond between TCR and peptide.

### Investigation of the Effects of Covalent Antigen Recognition on CD4+ T Cell Activation In Vitro and In Vivo Using the Engineered LS1-α3/DR15 Complex

Engineering a disulphide bond into the LS1-α3/DR15 complex would enable investigation of the effects of covalent antigen recognition on CD4+ T cell activation in vitro and in vivo. For example, T-reg function could be assessed in vitro, using methods akin to those described herein. To enable in vivo experiments, DR15-transgenic Fcgr2b^{-/-} mouse germline DNA encoding the α3 peptide could be modified to introduce a cysteine at the desired position using CRISPR-Cas9 gene editing. This approach has the benefit that the α3 protein and peptide are expressed and presented to T cells in a pattern that is pivotal to protection and susceptibility to autoimmune disease in humans and humanised mouse models. Such an approach may provide a new model to test whether TCR-Treg cells engineered to form a disulphide bond with target antigen confer superior efficacy in the treatment of autoimmune disease.

It will be understood that the invention disclosed and defined in this specification extends to all alternative combinations of two or more of the individual features mentioned or evident from the text or drawings. All of these different combinations constitute various alternative aspects of the invention.

By way of clarification and for avoidance of doubt, as used herein and except where the context requires otherwise, the term “comprise” and variations of the term, such as “comprising”, “comprises” and “comprised”, are not intended to exclude further additions, components, integers or steps.

TABLE 3
Potential disulphide bonds in TCR/peptide-MHC class I complexes
(see Example 1 for methods):

| | | | | | | | Mu- | |
|---|---|---|---|---|---|---|---|---|
| | | | | | | Mu- | table | |
| | | | | | Mu- | table | pep- | |
| | | | | | table | CDR3 | tide | |
| | | Peptide | | Indica- | TCR | posi- | posi- | Confi- |
| CDR3α | CDR3β | Sequence | MHC | tion | chain | tion | tion | dence |
| AVNFGGGKLI | ASSLSFGTEAF | AAGIGILTV | HLA- | Melanoma | β | 7 | 4 | high |
| (SEQ ID | (SEQ ID | (SEQ ID | A*02:01 | | | | | |
| NO: 21) | NO: 65) | NO: 107) | | | | | | |
| AVNFGGGKLI | ASSLSFGTEAF | AAGIGILTV | HLA- | Melanoma | β | 5 | 7 | high |
| (SEQ ID | (SEQ ID | (SEQ ID | A*02:01 | | | | | |
| NO: 21) | NO: 65) | NO: 107) | | | | | | |
| ALFLASSSFS | ASSDWVSYEQY | ALWGFFPVL | HLA- | Self- | α | 5 | 5 | high |
| KLV | (SEQ ID | (SEQ ID | A*02:01 | tolerance | | | | |
| (SEQ ID | NO: 66) | NO: 108) | | | | | | |
| NO: 22) | | | | | | | | |
| AMRGDSSYKL | ASSLWEKLAKN | ALWGPDPAA | HLA- | Self- | β | 5 | 8 | high |
| I | IQY | A | A*02:01 | tolerance | | | | |
| (SEQ ID | (SEQ ID | (SEQ ID | | | | | | |
| NO: 23) | NO: 67) | NO: 109) | | | | | | |
| LVGEILDNFN | ASSQRQEGDTQ | APRGPHGGA | HLA- | Cancer | β | 6 | 10 | low |
| K | Y | ASGL | B*07:02 | | | | | |
| (SEQ ID | (SEQ ID | (SEQ ID | | | | | | |
| NO: 24) | NO: 68) | NO: 110) | | | | | | |
| LVVDQKLV | ASSGGHTGSNE | APRGPHGGA | HLA- | Cancer | β | 10 | 6 | high |
| (SEQ ID | QF | ASGL | B*07:02 | | | | | |
| NO: 25) | (SEQ ID | (SEQ ID | | | | | | |
| | NO: 69) | NO: 110) | | | | | | |
| IVWGGYQKVT | ASRYRDDSYNE | EENLLDFVR | HLA- | EBV | α | 6 | 4 | high |
| (SEQ ID | QF | F | B*44:05 | | | | | |
| NO: 26) | (SEQ ID | (SEQ ID | | | | | | |
| | NO: 70) | NO: 111) | | | | | | |
| IVWGGYQKVT | ASRYRDDSYNE | EENLLDFVR | HLA- | EBV | α | 6 | 6 | high |
| (SEQ ID | QF | F | B*44:05 | | | | | |
| NO: 26) | (SEQ ID | (SEQ ID | | | | | | |
| | NO: 70) | NO: 111) | | | | | | |
| IVWGGYQKVT | ASRYRDDSYNE | EENLLDFVR | HLA- | EBV | β | 8 | 7 | high |
| (SEQ ID | QF | F | B*44:05 | | | | | |
| NO: 26) | (SEQ ID | (SEQ ID | | | | | | |
| | NO: 70) | NO: 111) | | | | | | |
| ILPLAGGTSY | ASSLGQAYEQY | EEYLQAFTY | HLA- | Self- | α | 4 | 4 | high |
| GKLT | (SEQ ID | (SEQ ID | B*44:05 | tolerance | | | | |
| (SEQ ID | NO: 71) | NO: 112) | | | | | | |
| NO: 27) | | | | | | | | |
| ILPLAGGTSY | ASSLGQAYEQY | EEYLQAFTY | HLA- | Self- | α | 7 | 6 | high |
| GKLT | (SEQ ID | (SEQ ID | B*44:05 | tolerance | | | | |
| (SEQ ID | NO: 71) | NO: 112) | | | | | | |
| NO: 27) | | | | | | | | |
| ILPLAGGTSY | ASSLGQAYEQY | EEYLQAFTY | HLA- | Self- | β | 7 | 7 | low |
| GKLT | (SEQ ID | (SEQ ID | B*44:05 | tolerance | | | | |
| (SEQ ID | NO: 71) | NO: 112) | | | | | | |
| NO: 27) | | | | | | | | |
| AVNVAGKST | AWSETGLGTGE | ELAGIGILT | HLA- | Melanoma | α | 3 | 4 | low |
| (SEQ ID | LF | V | A*02:01 | | | | | |
| NO: 28) | (SEQ ID | (SEQ ID | | | | | | |
| | NO: 72) | NO: 113) | | | | | | |
| AVNVAGKST | AWSETGLGTGE | ELAGIGILT | HLA- | Melanoma | β | 5 | 9 | high |
| (SEQ ID | LF | V | A*02:01 | | | | | |
| NO: 28) | (SEQ ID | (SEQ ID | | | | | | |
| | NO: 72) | NO: 113) | | | | | | |
| AVQASGGSYI | ATGTGDSNQPQ | EPLPQGQLT | HLA- | EBV | β | 4 | 7 | high |
| PT | H | AY | B*35:01 | | | | | |
| (SEQ ID | (SEQ ID | (SEQ ID | | | | | | |
| NO: 29) | NO: 73) | NO: 114) | | | | | | |
| AVQASGGSYI | ATGTGDSNQPQ | EPLPQGQLT | HLA- | EBV | β | 5 | 8 | high |
| PT | H | AY | B*35:01 | | | | | |
| (SEQ ID | (SEQ ID | (SEQ ID | | | | | | |
| NO: 29) | NO: 73) | NO: 114) | | | | | | |
| AVRPGGAGPF | ASSFNMATGQY | ESDPIVAQY | HLA- | Cancer | β | 5 | 5 | low |
| FVV | (SEQ ID | (SEQ ID | A*01:01 | | | | | |
| (SEQ ID | NO: 74) | NO: 115) | | | | | | |
| NO: 30) | | | | | | | | |
| AVRPGGAGPF | ASSFNMATGQY | EVDPIGHLY | HLA- | Cancer | β | 7 | 5 | high |
| FVV | (SEQ ID | (SEQ ID | A*01:01 | | | | | |
| (SEQ ID | NO: 74) | NO: 116) | | | | | | |
| NO: 30) | | | | | | | | |
| GTERSGGYQK | ASSMSAMGTEA | FEDLRVLSF | HLA- | Influenza | α | 9 | 4 | high |
| VT | F | (SEQ ID | B*37:01 | | | | | |
| (SEQ ID | (SEQ ID | NO: 117) | | | | | | |
| NO: 31) | NO: 75) | | | | | | | |
| GTERSGGYQK | ASSMSAMGTEA | FEDLRVLSF | HLA- | Influenza | β | 4 | 7 | high |
| VT | F | (SEQ ID | B*37:01 | | | | | |
| (SEQ ID | (SEQ ID | NO: 117) | | | | | | |
| NO: 31) | NO: 75) | | | | | | | |
| AMREDTGNFY | ASSFTWTSGGA | FLRGRAYGL | HLA- | EBV | β | 7 | 4 | high |
| (SEQ ID | TDTQY | (SEQ ID | B*08:01 | | | | | |
| NO: 32) | (SEQ ID | NO: 118) | | | | | | |
| | NO: 76) | | | | | | | |
| AMREDTGNFY | ASSFTWTSGGA | FLRGRAYGL | HLA- | EBV | β | 5 | 6 | high |
| (SEQ ID | TDTQY | (SEQ ID | B*08:01 | | | | | |
| NO: 32) | (SEQ ID | NO: 118) | | | | | | |
| | NO: 76) | | | | | | | |
| ILPLAGGTSY | ASSLGQAYEQY | FLRGRAYGL | HLA- | EBV | α | 5 | 4 | high |
| GKLT | (SEQ ID | (SEQ ID | B*08:01 | | | | | |
| (SEQ ID | NO: 77) | NO: 118) | | | | | | |
| NO: 33) | | | | | | | | |
| ILPLAGGTSY | ASSLGQAYEQY | FLRGRAYGL | HLA- | EBV | α | 4 | 6 | low |
| GKLT | (SEQ ID | (SEQ ID | B*08:01 | | | | | |
| (SEQ ID | NO: 77) | NO: 118) | | | | | | |
| NO: 33) | | | | | | | | |
| ILPLAGGTSY | ASSLGQAYEQY | FLRGRAYGL | HLA- | EBV | β | 7 | 6 | high |
| GKLT | (SEQ ID | (SEQ ID | B*08:01 | | | | | |
| (SEQ ID | NO: 77) | NO: 118) | | | | | | |
| NO: 33) | | | | | | | | |
| VVRAGKLI | ASGQGNFDIQY | FLRGRAYGL | HLA- | EBV | β | 8 | 6 | high |
| (SEQ ID | (SEQ ID | (SEQ ID | B*08:01 | | | | | |
| NO: 34) | NO: 78) | NO: 118) | | | | | | |
| AASFIIQGAQ | ASSLLGGWSEA | GILGFVFTL | HLA- | Influenza | α | 10 | 5 | high |
| KLV | F | (SEQ ID | A*02:01 | | | | | |
| (SEQ ID | (SEQ ID | NO: 119) | | | | | | |
| NO: 35) | NO: 79) | | | | | | | |
| AASFIIQGAQ | ASSLLGGWSEA | GILGFVFTL | HLA- | Influenza | β | 8 | 7 | high |
| KLV | F | (SEQ ID | A*02:01 | | | | | |
| (SEQ ID | (SEQ ID | NO: 119) | | | | | | |
| NO: 35) | NO: 79) | | | | | | | |
| AFDTNAGKST | ASSIFGQREQY | GILGFVFTL | HLA- | Influenza | β | 5 | 7 | high |
| (SEQ ID | (SEQ ID | (SEQ ID | A*02:01 | | | | | |
| NO: 36) | NO: 80) | NO: 119) | | | | | | |
| AGAGSQGNLI | ASSSRSSYEQY | GILGFVFTL | HLA- | Influenza | β | 7 | 5 | low |
| (SEQ ID | (SEQ ID | (SEQ ID | A*02:01 | | | | | |
| NO: 37) | NO: 81) | NO: 119) | | | | | | |
| AGAGSQGNLI | ASSSRSSYEQY | GILGFVFTL | HLA- | Influenza | β | 5 | 7 | high |
| (SEQ ID | (SEQ ID | (SEQ ID | A*02:01 | | | | | |
| NO: 37) | NO: 81) | NO: 119) | | | | | | |
| AWGVNAGGTS | ASSIGVYGYT | GILGFVFTL | HLA- | Influenza | α | 6 | 5 | high |
| YGKLT | (SEQ ID | (SEQ ID | A*02:01 | | | | | |
| (SEQ ID | NO: 82) | NO: 119) | | | | | | |
| NO: 38) | | | | | | | | |
| AWGVNAGGTS | ASSIGVYGYT | GILGFVFTL | HLA- | Influenza | β | 4 | 7 | high |
| YGKLT | (SEQ ID | (SEQ ID | A*02:01 | | | | | |
| (SEQ ID | NO: 82) | NO: 119) | | | | | | |
| NO: 38) | | | | | | | | |
| AEDNNARLM | SARDGTGNGYT | GLCTLVAML | HLA- | EBV | α | 3 | 4 | high |
| (SEQ ID | (SEQ ID | (SEQ ID | A*02:01 | | | | | |
| NO: 39) | NO: 83) | NO: 120) | | | | | | |
| AEDNNARLM | SARDGTGNGYT | GLCTLVAML | HLA- | EBV | β | 3 | 6 | low |
| (SEQ ID | (SEQ ID | (SEQ ID | A*02:01 | | | | | |
| NO: 39) | NO: 83) | NO: 120) | | | | | | |
| AEDNNARLM | SARDGTGNGYT | GLCTLVAML | HLA- | EBV | β | 8 | 7 | high |
| (SEQ ID | (SEQ ID | (SEQ ID | A*02:01 | | | | | |
| NO: 39) | NO: 83) | NO: 120) | | | | | | |
| GLGDAGNMLT | ASSLGQGLLYG | GTSGSPIVN | HLA- | Dengue | β | 8 | 5 | high |
| (SEQ ID | YT | R | A*11:01 | virus | | | | |
| NO: 40) | (SEQ ID | (SEQ ID | | | | | | |
| | NO: 84) | NO: 121) | | | | | | |
| AVQDLGTSGS | ASSARSGELF | HPVGEADYF | HLA- | EBV | α | 10 | 6 | high |
| RLT | (SEQ ID | EY | B*35:01 | | | | | |
| (SEQ ID | NO: 85) | (SEQ ID | | | | | | |
| NO: 41) | | NO: 122) | | | | | | |
| AVQDLGTSGS | ASSARSGELF | HPVGEADYF | HLA- | EBV | β | 3 | 6 | high |
| RLT | (SEQ ID | EY | B*35:01 | | | | | |
| (SEQ ID | NO: 85) | (SEQ ID | | | | | | |
| NO: 41) | | NO: 122) | | | | | | |
| AVQDLGTSGS | ASSARSGELF | HPVGEADYF | HLA- | EBV | β | 3 | 7 | high |
| RLT | (SEQ ID | EY | B*35:01 | | | | | |
| (SEQ ID | NO: 85) | (SEQ ID | | | | | | |
| NO: 41) | | NO: 122) | | | | | | |
| AVQDLGTSGS | ASSARSGELF | HPVGEADYF | HLA- | EBV | β | 5 | 8 | low |
| RLT | (SEQ ID | EY | B*35:01 | | | | | |
| (SEQ ID | NO: 85) | (SEQ ID | | | | | | |
| NO: 41) | | NO: 122) | | | | | | |
| ALSDPVNDMR | ASSLRGRGDQ | HSKKKCDEL | HLA- | HCV | β | 5 | 6 | high |
| (SEQ ID | PQH | (SEQ ID | B*08:01 | | | | | |
| NO: 42) | (SEQ ID | NO: 123) | | | | | | |
| | NO: 86) | | | | | | | |
| ALSDPVNDMR | ASSLRGRGDQ | HSKKKCDEL | HLA- | HCV | β | 7 | 6 | high |
| (SEQ ID | PQH | (SEQ ID | B*08:01 | | | | | |
| NO: 42) | (SEQ ID | NO: 123) | | | | | | |
| | NO: 86) | | | | | | | |
| AVDSATSGTY | ASSYQGTEAF | ILAKFLHWL | HLA- | Cancer | β | 6 | 6 | high |
| KYI | (SEQ ID | (SEQ ID | A*02:01 | | | | | |
| (SEQ ID | NO: 87) | NO: 124) | | | | | | |
| NO: 43) | | | | | | | | |
| ALGEGGAQKL | ASRTRGGTLI | IPLTEEAEL | HLA- | HIV | β | 5 | 5 | high |
| V | EQY | (SEQ ID | B*35:01 | | | | | |
| (SEQ ID | (SEQ ID | NO: 125) | | | | | | |
| NO: 44) | NO: 88) | | | | | | | |
| ALSHNSGGSN | ASSFRGGKTQ | IPLTEEAEL | HLA- | HIV | α | 11 | 4 | low |
| YKLT | Y | (SEQ ID | B*35:01 | | | | | |
| (SEQ ID | (SEQ ID | NO: 125) | | | | | | |
| NO: 45) | NO: 89) | | | | | | | |
| ALSHNSGGSN | ASSFRGGKTQ | IPLTEEAEL | HLA- | HIV | β | 5 | 5 | high |
| YKLT | Y | (SEQ ID | B*35:01 | | | | | |
| (SEQ ID | (SEQ ID | NO: 125) | | | | | | |
| NO: 45) | NO: 89) | | | | | | | |
| AYGEDDKII | ASRRGPYEQY | KLVALGINA | HLA- | HCV | α | 4 | 4 | high |
| (SEQ ID | (SEQ ID | V | A*02:01 | | | | | |
| NO: 46) | NO: 90) | (SEQ ID | | | | | | |
| | | NO: 126) | | | | | | |
| AMRDLRDNFN | ASREGLGGTEA | KRWIILGLN | HLA- | HIV | α | 9 | 6 | low |
| KFY | F | K | B*27:05 | | | | | |
| (SEQ ID | (SEQ ID | (SEQ ID | | | | | | |
| NO: 47) | NO: 91) | NO: 127) | | | | | | |
| AMRDLRDNFN | ASREGLGGTEA | KRWIILGLN | HLA- | HIV | β | 9 | 6 | high |
| KFY | F | K | B*27:05 | | | | | |
| (SEQ ID | (SEQ ID | (SEQ ID | | | | | | |
| NO: 47) | NO: 92) | NO: 127) | | | | | | |
| AMRDLRDNFN | ASREGLGGTEA | KRWIILGLN | HLA- | HIV | β | 4 | 9 | high |
| KFY | F | K | B*27:05 | | | | | |
| (SEQ ID | (SEQ ID | (SEQ ID | | | | | | |
| NO: 47) | NO: 92) | NO: 127) | | | | | | |
| AGGTGNQFY | AISEVGVGQPQ | LAGIGILTV | HLA- | Melanoma | α | 5 | 4 | low |
| (SEQ ID | H | (SEQ ID | A*02:01 | | | | | |
| NO: 48) | (SEQ ID | NO: 128) | | | | | | |
| | NO: 93) | | | | | | | |
| AGGTGNQFY | AISEVGVGQPQ | LAGIGILTV | HLA- | Melanoma | β | 7 | 7 | high |
| (SEQ ID | H | (SEQ ID | A*02:01 | | | | | |
| NO: 48) | (SEQ ID | NO: 128) | | | | | | |
| | NO: 93) | | | | | | | |
| AGGTGNQFY | AISEVGVGQPQ | LAGIGILTV | HLA- | Melanoma | β | 5 | 8 | high |
| (SEQ ID | H | (SEQ ID | A*02:01 | | | | | |
| NO: 48) | (SEQ ID | NO: 128) | | | | | | |
| | NO: 93) | | | | | | | |
| AVTTDSWGK | ASRPGLAGGRP | LLFGYPVYV | HLA- | Leukaemia | α | 5 | 4 | high |
| LQ | EQY | (SEQ ID | A*02:01 | | | | | |
| (SEQ ID | (SEQ ID | NO: 129) | | | | | | |
| NO: 49) | NO: 94) | | | | | | | |
| ALSGFYNTD | ASPGLAGEYEQ | LPEPLPQGQ | HLA- | EBV | α | 7 | 5 | high |
| KLI | Y | LTAY | B*35:08 | | | | | |
| (SEQ ID | (SEQ ID | (SEQ ID | | | | | | |
| NO: 50) | NO: 95) | NO: 130) | | | | | | |
| AVGGGSNYQ | ASSRTGSTYEQ | LPEPLPQGQ | HLA- | EBV | β | 7 | 5 | high |
| LI | Y | LTAY | B*35:08 | | | | | |
| (SEQ ID | (SEQ ID | (SEQ ID | | | | | | |
| NO: 51) | NO: 96) | NO: 130) | | | | | | |
| AVGGGSNYQ | ASSRTGSTYEQ | LPEPLPQGQ | HLA- | EBV | β | 8 | 7 | high |
| LI | Y | LTAY | B*35:08 | | | | | |
| (SEQ ID | (SEQ ID | (SEQ ID | | | | | | |
| NO: 51) | NO: 96) | NO: 130) | | | | | | |
| AVNFGGGKL | ASSLSFGTEAF | MMWDRGLGM | HLA- | Synthetic | β | 5 | 5 | low |
| I | (SEQ ID | M | A*02:01 | | | | | |
| (SEQ ID | NO: 65) | (SEQ ID | | | | | | |
| NO: 52) | | NO: 131) | | | | | | |
| AMRGDSSYKL | ASSLWEKLAKN | MVWGPDPLY | HLA- | Diabetes | β | 5 | 6 | high |
| I | IQY | V | A*02:01 | | | | | |
| (SEQ ID | (SEQ ID | (SEQ ID | | | | | | |
| NO: 53) | NO: 67) | NO: 132) | | | | | | |
| AMRGDSSYKL | ASSLWEKLAKN | MVWGPDPLY | HLA- | Diabetes | β | 5 | 8 | high |
| I | IQY | V | A*02:01 | | | | | |
| (SEQ ID | (SEQ ID | (SEQ ID | | | | | | |
| NO: 53) | NO: 67) | NO: 132) | | | | | | |
| AFITGNQFY | ASSQTQLWETQ | NLVPMVATV | HLA- | Cytomega- | α | 5 | 5 | low |
| (SEQ ID | Y | (SEQ ID | A*02:01 | lovirus | | | | |
| NO: 54) | (SEQ ID | NO: 133) | | | | | | |
| | NO: 97) | | | | | | | |
| ILDNNNDMR | ASSLAPGTTNE | NLVPMVATV | HLA- | Cytomega- | β | 8 | 5 | low |
| (SEQ ID | KLF | (SEQ ID | A*02:01 | lovirus | | | | |
| NO: 55) | (SEQ ID | NO: 133) | | | | | | |
| | NO: 98) | | | | | | | |
| AVGAPSGAGS | ASSPTSGIYEQ | RFPLTFGWC | HLA- | HIV | α | 11 | 6 | low |
| YQLT | Y | F | A*24:02 | | | | | |
| (SEQ ID | (SEQ ID | (SEQ ID | | | | | | |
| NO: 56) | NO: 99) | NO: 134) | | | | | | |
| AVGAPSGAGS | ASSPTSGIYEQ | RFPLTFGWC | HLA- | HIV | β | 6 | 9 | high |
| YQLT | Y | F | A*24:02 | | | | | |
| (SEQ ID | (SEQ ID | (SEQ ID | | | | | | |
| NO: 56) | NO: 99) | NO: 134) | | | | | | |
| WGTYNQGGKL | ASSGASHEQY | RFPLTFGWC | HLA- | HIV | α | 4 | 4 | low |
| I | (SEQ ID | F | A*24:02 | | | | | |
| (SEQ ID | NO: 100) | (SEQ ID | | | | | | |
| NO: 57) | | NO: 134) | | | | | | |
| WGTYNQGGKL | ASSGASHEQY | RFPLTFGWC | HLA- | HIV | β | 7 | 4 | low |
| I | (SEQ ID | F | A*24:02 | | | | | |
| (SEQ ID | NO: 100) | (SEQ ID | | | | | | |
| NO: 57) | | NO: 134) | | | | | | |
| AMRGDSSYKL | ASSLWEKLAKN | RQFGPDFPT | HLA- | Diabetes | β | 5 | 6 | low |
| I | IQY | I | A*02:01 | | | | | |
| (SEQ ID | (SEQ ID | (SEQ ID | | | | | | |
| NO: 58) | NO: 67) | NO: 135) | | | | | | |
| AMRGDSSYKL | ASSLWEKLAKN | RQFGPDWIV | HLA- | Diabetes | β | 5 | 8 | high |
| I | IQY | A | A*02:01 | | | | | |
| (SEQ ID | (SEQ ID | (SEQ ID | | | | | | |
| NO: 58) | NO: 67) | NO: 136) | | | | | | |
| AMRGDSSYKL | ASSLWEKLAKN | RQWGPDPAA | HLA- | Diabetes | β | 5 | 8 | low |
| I | IQY | V | A*02:01 | | | | | |
| (SEQ ID | (SEQ ID | (SEQ ID | | | | | | |
| NO: 58) | NO: 67) | NO: 137) | | | | | | |
| AVRMDSSYKL | ASSSWDTGELF | RYPLTFGWC | HLA- | HIV | β | 8 | 6 | high |
| I | (SEQ ID | F | A*24:02 | | | | | |
| (SEQ ID | NO: 101) | (SEQ ID | | | | | | |
| NO: 58) | | NO: 138) | | | | | | |
| AVRMDSSYKL | ASSSWDTGELF | RYPLTFGWC | HLA- | HIV | β | 6 | 7 | low |
| I | (SEQ ID | F | A*24:02 | | | | | |
| (SEQ ID | NO: 101) | (SEQ ID | | | | | | |
| NO: 58) | | NO: 138) | | | | | | |
| AVRMDSSYKL | ASSSWDTGELF | RYPLTFGWC | HLA- | HIV | β | 5 | 9 | high |
| I | (SEQ ID | F | A*24:02 | | | | | |
| (SEQ ID | NO: 101) | (SEQ ID | | | | | | |
| NO: 58) | | NO: 138) | | | | | | |
| AVKSGGSYIP | ASSYLNRDSAL | SLLMWITQV | HLA- | Cancer | β | 6 | 7 | high |
| T | D | (SEQ ID | A*02:01 | | | | | |
| (SEQ ID | (SEQ ID | NO: 139) | | | | | | |
| NO: 59) | NO: 102) | | | | | | | |
| AVRTNSGYAL | ASSDTVSYEQY | SLYNTVATL | HLA- | HIV | β | 8 | 5 | high |
| N | (SEQ ID | (SEQ ID | A*02:01 | | | | | |
| (SEQ ID | NO: 103) | NO: 140) | | | | | | |
| NO: 60) | | | | | | | | |
| AVRTNSGYAL | ASSDTVSYEQY | SLYNTVATL | HLA- | HIV | β | 5 | 8 | high |
| N | (SEQ ID | (SEQ ID | A*02:01 | | | | | |
| (SEQ ID | NO: 103) | NO: 140) | | | | | | |
| NO: 60) | | | | | | | | |
| AVNFGGGKLI | ASSLSFGTEAF | SMLGIGIVP | HLA- | Synthetic | β | 5 | 8 | high |
| (SEQ ID | (SEQ ID | V | A*02:01 | | | | | |
| NO: 61) | NO: 65) | (SEQ ID | | | | | | |
| | | NO: 141) | | | | | | |
| ATDDDSARQL | ASSLTGGGELF | TAFTIPSI | HLA- | HIV | α | 8 | 4 | high |
| T | (SEQ ID | (SEQ ID | B*51:01 | | | | | |
| (SEQ ID | NO: 104) | NO: 142) | | | | | | |
| NO: 62) | | | | | | | | |
| AGQPLGGSNY | ASSANPGDSSN | VMAPRTLIL | HLA- | Cytomega- | α | 9 | 8 | high |
| KLT | EKLF | (SEQ ID | E*01:03 | lovirus | | | | |
| (SEQ ID | (SEQ ID | NO: 143) | | | | | | |
| NO: 63) | NO: 105) | | | | | | | |
| IVVRSSNTGK | ASSQDRDTQY | VMAPRTLIL | HLA- | Cytomega- | α | 5 | 5 | high |
| LI | (SEQ ID | (SEQ ID | E*01:03 | lovirus | | | | |
| (SEQ ID | NO: 106) | NO: 143) | | | | | | |
| NO: 64) | | | | | | | | |

TABLE 4
Potential disulphide bonds in TCR/peptide-MHC class Il complexes:

| | | | | | | | Mu- | |
|---|---|---|---|---|---|---|---|---|
| | | | | | | Mu- | table | |
| | | Peptide | | | Mu- | table | pep- | |
| | | sequence | | | table | CDR3 | tide | Con- |
| | | (P1 under- | MHC | Indica- | TCR | posi- | posi- | fi- |
| CDR3α | CDR3β | lined) | allele(s) | tion | chain | tion | tion | dence |
| IAFQGAQK | ASSFRALA | APQPELPY | HLA-DQA1* | Coeliac | β | 8 | 5 | high |
| LV | ADTQY | PQPGS | 0508_ | disease | | | | |
| (SEQ ID | (SEQ ID | (SEQ ID | HLA- | | | | | |
| NO: 144) | NO: 162) | NO: 181) | DQB1*0201 | | | | | |
| LVGDGGSF | SAGVGGQE | QPFPQPEL | HLA-DQA1* | Coeliac | β | 4 | 7 | low |
| SGGYNKLI | YTQ | PYP | 0501_ | disease | | | | |
| (SEQ ID | (SEQ ID | (SEQ ID | HLA-DQB1* | | | | | |
| NO: 145) | NO: 163) | NO: 182) | 0201 | | | | | |
| ILRDGRGG | ASSVAVSA | SGEGSFQP | HLA-DQA1* | Coeliac | α | 6 | 5 | high |
| ADGLT | GTYEQY | SQENP | 0301_ | disease | | | | |
| (SEQ ID | (SEQ ID | (SEQ ID | HLA-DQB1* | | | | | |
| NO: 146) | NO: 164) | NO: 183) | 0302 | | | | | |
| ILRDGRGG | ASSVAVSA | SGEGSFQP | HLA-DQA1* | Coeliac | β | 5 | 8 | high |
| ADGLT | GTYEQY | SQENP | 0301_ | disease | | | | |
| (SEQ ID | (SEQ ID | (SEQ ID | HLA-DQB1* | | | | | |
| NO: 146) | NO: 164) | NO: 183) | 0302 | | | | | |
| AVDRGSTL | ASSQIRET | FELIGILN | HLA-DRA* | Cancer | α | 6 | −1 | high |
| GRLY | QY | AAKVPAD | 0101_ | | | | | |
| (SEQ ID | (SEQ ID | (SEQ ID | HLA-DBR1* | | | | | |
| NO: 147) | NO: 165) | NO: 184) | 0101 | | | | | |
| AVDRGSTL | ASSQIRET | FELIGILN | HLA-DRA* | Cancer | α | 8 | 5 | high |
| GRLY | QY | AAKVPAD | 0101_ | | | | | |
| (SEQ ID | (SEQ ID | (SEQ ID | HLA-DBR1* | | | | | |
| NO: 147) | NO: 165) | NO: 184) | 0101 | | | | | |
| AVDRGSTL | ASSQIRET | FELIGILN | HLA-DRA* | Cancer | β | 6 | 5 | high |
| GRLY | QY | AAKVPAD | 0101_ | | | | | |
| (SEQ ID | (SEQ ID | (SEQ ID | HLA-DBR1* | | | | | |
| NO: 147) | NO: 165) | NO: 184) | 0101 | | | | | |
| AALIQGAQ | ASTYHGTG | GELIGTLN | HLA-DRA* | Cancer | α | 5 | −1 | high |
| KLV | Y | AAKVPAD | 0101_ | | | | | |
| (SEQ ID | (SEQ ID | (SEQ ID | HLA-DBR1* | | | | | |
| NO: 148) | NO: 166) | NO: 185) | 0101 | | | | | |
| AVSESPFG | ASSSTGLP | PKYVKQNT | HLA-DRA* | Influenza | β | 5 | 5 | high |
| NEKLT | YGYT | LKLAT | 0101_ | | | | | |
| (SEQ ID | (SEQ ID | (SEQ ID | HLA-DBR1* | | | | | |
| NO: 149) | NO: 167) | NO: 186) | 0101 | | | | | |
| AVGASGNT | ASSLRDGY | QHIRCNIP | HLA-DRA* | Nickel | β | 5 | 5 | high |
| GKLI | TGELF | KRISA | 0101_ | hypersen- | | | | |
| (SEQ ID | (SEQ ID | (SEQ ID | HLA-DRB3* | sitivity | | | | |
| NO: 150) | NO: 168) | NO: 187) | 0301 | | | | | |
| AVGASGNT | ASSLRDGY | QHIRCNIP | HLA-DRA* | Nickel | β | 6 | 8 | high |
| GKLI | TGELF | KRISA | 0101_ | hypersen- | | | | |
| (SEQ ID | (SEQ ID | (SEQ ID | HLA-DRB3* | sitivity | | | | |
| NO: 150) | NO: 168) | NO: 187) | 0301 | | | | | |
| ALSGGDSS | ASSLADRV | VHFFKNIV | HLA-DRA* | Multiple | β | 8 | 4 | high |
| YKLI | NTEAF | TPRTPGG | 0101_ | sclerosis | | | | |
| (SEQ ID | (SEQ ID | (SEQ ID | HLA-DBR5* | | | | | |
| NO: 150) | NO: 169) | NO: 188) | 0101 | | | | | |
| TVYGGATN | SARGGSYN | FSWGAEGQ | HLA-DRA* | Multiple | α | 6 | −1 | high |
| KLI | SPLH | RPGFGSGG | 0101_ | sclerosis | | | | |
| (SEQ ID | (SEQ ID | (SEQ ID | HLA-DBR1* | | | | | |
| NO: 151) | NO: 170) | NO: 189) | 0401 | | | | | |
| TVYGGATN | SARGGSYN | FSWGAEGQ | HLA-DRA* | Multiple | β | 9 | 7 | high |
| KLI | SPLH | RPGFGSGG | 0101_ | sclerosis | | | | |
| (SEQ ID | (SEQ ID | (SEQ ID | HLA-DBR1* | | | | | |
| NO: 151) | NO: 170) | NO: 189) | 0401 | | | | | |
| ALSLYSGA | ASSLAQGG | QAFWIDLF | HLA-DPA1* | Beryllium | α | 5 | 2 | low |
| GSYQLT | ETQY | ETIG | 0103_ | hypersen- | | | | |
| (SEQ ID | (SEQ ID | (SEQ ID | HLA-DPB1* | sitivity | | | | |
| NO: 152) | NO: 171) | NO: 190) | 2602 | | | | | |
| ALSLYSGA | ASSLAQGG | QAFWIDLF | HLA-DPA1* | Beryllium | β | 6 | 5 | high |
| GSYQLT | ETQY | ETIG | 0103_ | hypersen- | | | | |
| (SEQ ID | (SEQ ID | (SEQ ID | HLA-DPB1* | sitivity | | | | |
| NO: 152) | NO: 171) | NO: 190) | 2602 | | | | | |
| ILRDRSNQ | ASSTTPGT | APSGEGSF | HLA-DQA1* | Coeliac | α | 5 | 5 | high |
| FY | GTETQY | QPSQENPQ | 0301_ | disease | | | | |
| (SEQ ID | (SEQ ID | GS | HLA-DQB1* | | | | | |
| NO: 153) | NO: 172) | (SEQ ID | 0302 | | | | | |
| | | NO: 191) | | | | | | |
| ILRDRSNQ | ASSTTPGT | APSGEGSF | HLA-DQA1* | Coeliac | β | 5 | 8 | high |
| FY | GTETQY | QPSQENPQ | 0301_ | disease | | | | |
| (SEQ ID | (SEQ ID | GS | HLA-DQB1* | | | | | |
| NO: 153) | NO: 172) | (SEQ ID | 0302 | | | | | |
| | | NO: 191) | | | | | | |
| ILRDSRAQ | ASSAGTSG | APSGEGSF | HLA-DQA1* | Coeliac | α | 6 | 5 | high |
| KLV | EYEQY | QPSQENPQ | 0301_ | disease | | | | |
| (SEQ ID | (SEQ ID | GS | HLA-DQB1* | | | | | |
| NO: 154) | NO: 173) | (SEQ ID | 0302 | | | | | |
| | | NO: 191) | | | | | | |
| ILRDSRAQ | ASSAGTSG | APSGEGSF | HLA-DQA1* | Coeliac | β | 7 | 5 | high |
| KLV | EYEQY | QPSQENPQ | 0301_ | disease | | | | |
| (SEQ ID | (SEQ ID | GS | HLA-DQB1* | | | | | |
| NO: 154) | NO: 173) | (SEQ ID | 0302 | | | | | |
| | | NO: 191) | | | | | | |
| AVGETGAN | ASSEARRY | APSGEGSF | HLA-DQA1* | Coeliac | α | 5 | 2 | high |
| NLF | NEQF | QPSQENPQ | 0301_ | disease | | | | |
| (SEQ ID | (SEQ ID | GS | HLA-DQB1* | | | | | |
| NO: 155) | NO: 174) | (SEQ ID | 0302 | | | | | |
| | | NO: 191) | | | | | | |
| AVGETGAN | ASSEARRY | APSGEGSF | HLA-DQA1* | Celiac | β | 4 | 8 | low |
| NLF | NEQF | QPSQENPQ | 0301_ | disease | | | | |
| (SEQ ID | (SEQ ID | GS | HLA-DQB1* | | | | | |
| NO: 155) | NO: 174) | (SEQ ID | 0302 | | | | | |
| | | NO: 191) | | | | | | |
| AVALNNNA | ASSVAPGS | APSGEGSF | HLA-DQA1* | Coeliac | β | 5 | 8 | high |
| GNMLT | DTQY | QPSQENPQ | 0301_ | disease | | | | |
| (SEQ ID | (SEQ ID | (SEQ ID | HLA-DQB1* | | | | | |
| NO: 156) | NO: 175) | NO: 192) | 0302 | | | | | |
| AVALNNNA | ASSVAPGS | QPQQSFPE | HLA-DQA1* | Coeliac | α | 11 | 2 | high |
| GNMLT | DTQY | QEA | 0501_ | disease | | | | |
| (SEQ ID | (SEQ ID | (SEQ ID | HLA-DQB1* | | | | | |
| NO: 156) | NO: 175) | NO: 193) | 0302 | | | | | |
| AVALNNNA | ASSVAPGS | QPQQSFPE | HLA-DQA1* | Coeliac | β | 5 | 8 | high |
| GNMLT | DTQY | QEA | 0501_ | disease | | | | |
| (SEQ ID | (SEQ ID | (SEQ ID | HLA-DQB1* | | | | | |
| NO: 156) | NO: 175) | NO: 193) | 0302 | | | | | |
| AVQASGGS | ASSNRGLG | GPQQSFPE | HLA-DQA1* | Coeliac | β | 5 | 8 | high |
| YIPT | TDTQY | QEA | 0501_ | disease | | | | |
| (SEQ ID | (SEQ ID | (SEQ ID | HLA-DQB1* | | | | | |
| NO: 157) | NO: 176) | NO: 194) | 0302 | | | | | |
| AVQASGGS | ASSNRGLG | GPQQSFPE | HLA-DQA1* | Coeliac | β | 7 | 5 | high |
| YIPT | TDTQY | QEA | 0501_ | disease | | | | |
| (SEQ ID | (SEQ ID | (SEQ ID | HLA-DQB1* | | | | | |
| NO: 157) | NO: 176) | NO: 194) | 0302 | | | | | |
| AFKAAGNK | ASSRLAGG | RFYKTLRA | HLA-DRA* | HIV | β | 5 | 7 | high |
| LT | MDEQF | EQAS | 0101_ | | | | | |
| (SEQ ID | (SEQ ID | (SEQ ID | HLA-DRB1* | | | | | |
| NO: 158) | NO: 177) | NO: 195) | 1101 | | | | | |
| AFKAAGNK | ASSRLAGG | RFYKTLRA | HLA-DRA* | HIV | β | 6 | 8 | high |
| LT | MDEQF | EQAS | 0101_ | | | | | |
| (SEQ ID | (SEQ ID | (SEQ ID | HLA-DRB1* | | | | | |
| NO: 158) | NO: 177) | NO: 195) | 1101 | | | | | |
| AFKAAGNK | ASSRLAGG | RFYKTLRA | HLA-DRA* | HIV | β | 5 | 7 | high |
| LT | MDEQF | EQAS | 0101_ | | | | | |
| (SEQ ID | (SEQ ID | (SEQ ID | HLA-DRB1* | | | | | |
| NO: 158) | NO: 177) | NO: 195) | 1502 | | | | | |
| AFKAAGNK | ASSRLAGG | RFYKTLRA | HLA-DRA* | HIV | β | 6 | 8 | high |
| LT | MDEQF | EQAS | 0101_ | | | | | |
| (SEQ ID | (SEQ ID | (SEQ ID | HLA-DRB1* | | | | | |
| NO: 158) | NO: 177) | NO: 195) | 1502 | | | | | |
| AFKAAGNK | ASSRLAGG | RFYKTLRA | HLA-DRA* | HIV | β | 5 | 7 | high |
| LT | MDEQF | EQAS | 0101_ | | | | | |
| (SEQ ID | (SEQ ID | (SEQ ID | HLA-DRB1* | | | | | |
| NO: 158) | NO: 177) | NO: 195) | 0101 | | | | | |
| AFKAAGNK | ASSRLAGG | RFYKTLRA | HLA-DRA* | HIV | β | 6 | 8 | high |
| LT | MDEQF | EQAS ( | 0101_ | | | | | |
| (SEQ ID | (SEQ ID | SEQ ID | HLA-DRB1* | | | | | |
| NO: 158) | NO: 177) | NO: 195) | 0101 | | | | | |
| ATDAGYNQ | ASSAGNTI | VEELYLVA | HLA-DQA1* | Diabetes | α | 7 | 5 | high |
| GGKLI | Y | GEEGCGGG | 0301_ | | | | | |
| (SEQ ID | (SEQ ID | GSL | HLA-DQB1* | | | | | |
| NO: 159) | NO: 178) | (SEQ ID | 0305 | | | | | |
| | | NO: 196) | | | | | | |
| AASDTGAN | ASGDFWGD | RVSYYGPK | H2-Aa_ | Self- | β | 6 | 5 | high |
| TGKLT | TLY | TSPVQ | H2-Ab1 | tolerance | | | | |
| (SEQ ID | (SEQ ID | (SEQ ID | | | | | | |
| NO: 160) | NO: 179) | NO: 197) | | | | | | |
| ASPQGGSE | ASSSGGWG | APFSEQEQ | HLA-DQA1* | Coeliac | β | 7 | 5 | high |
| KLV | GGTEAF | PVLG | 0201_ | | | | | |
| (SEQ ID | (SEQ ID | (SEQ ID | HLA-DQB1* | | | | | |
| NO: 161) | NO: 180) | NO: 198) | 0201 | disease | | | | |

#For peptides presented by MHC class I, the first amino acid shown is designated P1.
*Residue pairs in bold and underlined text may form a disulphide bond if exchanged for cysteine; residue pairs in bold text are predicted to form a disulphide bond if exchanged for cysteine

1. A binding protein comprising a variable domain comprising a complementarity-determining region (CDR) capable of contacting a peptide bound to a HLA molecule, wherein the CDR comprises a cysteine capable of forming a disulphide bond with a cysteine in the peptide bound to the HLA molecule, wherein the cysteine is introduced into the CDR by mutation or modification of an existing residue.

2-3. (canceled)

4. The binding protein of claim 1, wherein the antigen binding protein is a T cell receptor or fragment thereof.

5. The binding protein of claim 1, wherein the antigen binding protein is a chimeric or fusion protein.

6. The binding protein of claim 1, wherein the chimeric protein is a chimeric antigen receptor (CAR).

7. The binding protein of claim 1, wherein the CDR is a CDR3.

8. The binding protein of claim 1, wherein the binding protein comprises an α chain variable domain (Vα) or a β chain variable domain (Vβ), and wherein the CDR containing the cysteine is present in the α chain variable domain.

9. (canceled)

10. The binding protein of claim 1, wherein the binding protein comprises an α chain variable domain (Vα) or a β chain variable domain (Vβ), wherein the CDR containing the cysteine is present in the 3 chain variable domain.

11. (canceled)

12. The binding protein of claim 1, wherein the HLA is a HLA class I.

13-14. (canceled)

15. The binding protein of claim 1, wherein the HLA is a HLA class II.

16. (canceled)

17. The binding protein of claim 1, wherein the cysteine capable of forming a disulphide bond with a cysteine in a peptide bound to a HLA molecule is present in the CDR at a position 3, 4, 5, 6, 7, 8, 9 or 10, wherein the numbering is relative to the amino acid at the N-terminus of the CDR (i.e. the amino acid at the N-terminus of the CDR is position 1).

18. The binding protein of claim 1, wherein the CDR is present in a TCR α-chain variable domain, the cysteine is present in the CDR at a position 3, 4, 5, 6, 7, 8, 9 or 10 wherein the numbering is relative to the amino acid at the N-terminus of the CDR (i.e. the amino acid at the N-terminus of the CDR is position 1) thereby allowing formation of a disulphide bond with a cysteine present in a peptide bound to a HLA class I molecule.

19. The binding protein of claim 1, wherein the CDR is present in a TCR β-chain variable domain, the cysteine is present in the CDR at a position 3, 4, 5, 6, 7, 8, 9 or 10 wherein the numbering is relative to the amino acid at the N-terminus of the CDR (i.e. the amino acid at the N-terminus of the CDR is position 1) thereby allowing formation of a disulphide bond with a cysteine present in a peptide bound to a HLA class I molecule.

20. The binding protein of claim 1, wherein the CDR is present in a TCR α-chain variable domain, the cysteine is present in the CDR at a position 5, 6, 7, 8 or 11 wherein the numbering is relative to the amino acid at the N-terminus of the CDR (i.e. the amino acid at the N-terminus of the CDR is position 1) thereby allowing formation of a disulphide bond with a cysteine present in a peptide bound to a HLA class II molecule.

21. The binding protein of claim 1, wherein the CDR is present in a TCR β-chain variable domain, the cysteine is present in the CDR at a position 5, 6, 7, 8 or 9 wherein the numbering is relative to the amino acid at the N-terminus of the CDR (i.e. the amino acid at the N-terminus of the CDR is position 1) thereby allowing formation of a disulphide bond with a cysteine present in a peptide bound to a HLA class II molecule.

22-27. (canceled)

28. A vector comprising a nucleic acid comprising, consisting essentially of or consisting of a nucleotide sequence encoding a binding protein of claim 1.

29. (canceled)

30. A cell expressing on its surface a binding protein of claim 1.

31-35. (canceled)

36. The cell of claim 30, wherein the cell is a T regulatory cell.

37. A method of preparing a population of T regulatory cells, the method comprising:
providing a population of T regulatory cells,
introducing a vector of claim 28 into the population of T regulatory cells, and
providing conditions to allow the expression of the binding protein on the surface of the T regulatory cells,
thereby preparing a population of T regulatory cells for use in the treatment of an autoimmune disease.

38-39. (canceled)

40. A method of treating or preventing an autoimmune disease, treating or preventing transplant rejection or treating or preventing cancer or an infectious disease, in a subject, the method comprising administering to the subject a binding protein of claim 1, thereby treating or preventing the autoimmune disease, treating or preventing transplant rejection or treating or preventing cancer or an infectious disease, in the subject.

41-48. (canceled)

49. A method of identifying a mutant TCR with a decreased rate of dissociation with its target peptide bound to an HLA (pHLA) compared to the unmutated TCR, the method comprising:
creating a plurality of TCRs having mutations to introduce a cysteine residue in an α chain CDR3 sequence and/or a β chain CDR3 sequence,
determining the interactions of members of said plurality of TCRs with the target pHLA, and
selecting one or more members having a decreased rate of dissociation with the target pHLA compared to the unmutated TCR,
wherein the decreased rate of dissociation is due to formation of a disulphide bond.

50. (canceled)


SEQUENCE LISTING (226 sequences)

SEQ ID NO: 1 - protein, 10 aa, Synthetic construct
LIKCDERGKM

SEQ ID NO: 2 - protein, 10 aa, Synthetic construct
GCLFDRRLCS

SEQ ID NO: 3 - protein, 10 aa, Synthetic construct
EYKIADPICT

SEQ ID NO: 4 - protein, 10 aa, Synthetic construct
EFRNMVTRCN

SEQ ID NO: 5 - protein, 10 aa, Synthetic construct
CLLLLSSRPG

SEQ ID NO: 6 - protein, 10 aa, Synthetic construct
EWQALCAYQA

SEQ ID NO: 7 - protein, 10 aa, Synthetic construct
AYQAEPNTCA

SEQ ID NO: 8 - protein, 10 aa, Synthetic construct
ELRKFSSLCY

SEQ ID NO: 9 - protein, 10 aa, Synthetic construct
PVLLLESHCA

SEQ ID NO: 10 - protein, 10 aa, Synthetic construct
IICYNWLHRR

SEQ ID NO: 11 - protein, 10 aa, Synthetic construct
RFRCQPGFVM

SEQ ID NO: 12 - protein, 10 aa, Synthetic construct
RLMMLQSCSG

SEQ ID NO: 13 - protein, 10 aa, Synthetic construct
CVRTMSALWN

SEQ ID NO: 14 - protein, 10 aa, Synthetic construct
ILRSMCGYQT

SEQ ID NO: 15 - protein, 9 aa, Homo sapiens
EGDCAPEEK

SEQ ID NO: 16 - protein, 9 aa, Homo sapiens
NYKRCFPVI

SEQ ID NO: 17 - protein, 9 aa, Homo sapiens
MLAVISCAV

SEQ ID NO: 18 - protein, 10 aa, Homo sapiens
EYSKECLKEF

SEQ ID NO: 19 - protein, 9 aa, Homo sapiens
RLSSCVPVA

SEQ ID NO: 20 - protein, 9 aa, Homo sapiens
FLDRFLSCM

SEQ ID NO: 21 - protein, 10 aa, Synthetic construct
AVNFGGGKLI

SEQ ID NO: 22 - protein, 13 aa, Synthetic construct
ALFLASSSFSKLV

SEQ ID NO: 23 - protein, 11 aa, Synthetic construct
AMRGDSSYKLI

SEQ ID NO: 24 - protein, 11 aa, Synthetic construct
LVGEILDNFNK

SEQ ID NO: 25 - protein, 8 aa, Synthetic construct
LVVDQKLV

SEQ ID NO: 26 - protein, 10 aa, Synthetic construct
IVWGGYQKVT

SEQ ID NO: 27 - protein, 14 aa, Synthetic construct
ILPLAGGTSYGKLT

SEQ ID NO: 28 - protein, 9 aa, Synthetic construct
AVNVAGKST

SEQ ID NO: 29 - protein, 9 aa, Synthetic construct
AVNVAGKST

SEQ ID NO: 30 - protein, 12 aa, Synthetic construct
AVQASGGSYIPT

SEQ ID NO: 31 - protein, 13 aa, Synthetic construct
AVRPGGAGPFFVV

SEQ ID NO: 32 - protein, 12 aa, Synthetic construct
GTERSGGYQKVT

SEQ ID NO: 33 - protein, 10 aa, Synthetic construct
AMREDTGNFY

SEQ ID NO: 34 - protein, 14 aa, Synthetic construct
ILPLAGGTSYGKLT

SEQ ID NO: 35 - protein, 8 aa, Synthetic construct
VVRAGKLI

SEQ ID NO: 36 - protein, 13 aa, Synthetic construct
AASFIIQGAQKLV

SEQ ID NO: 37 - protein, 10 aa, Synthetic construct
AFDTNAGKST

SEQ ID NO: 38 - protein, 10 aa, Synthetic construct
AGAGSQGNLI

SEQ ID NO: 39 - protein, 15 aa, Synthetic construct
AWGVNAGGTSYGKLT

SEQ ID NO: 40 - protein, 9 aa, Synthetic construct
AEDNNARLM

SEQ ID NO: 41 - protein, 10 aa, Synthetic construct
GLGDAGNMLT

SEQ ID NO: 42 - protein, 13 aa, Synthetic construct
AVQDLGTSGSRLT

SEQ ID NO: 43 - protein, 10 aa, Synthetic construct
ALSDPVNDMR

SEQ ID NO: 44 - protein, 13 aa, Synthetic construct
AVDSATSGTYKYI

SEQ ID NO: 45 - protein, 11 aa, Synthetic construct
ALGEGGAQKLV

SEQ ID NO: 46 - protein, 14 aa, Synthetic construct
ALSHNSGGSNYKLT

SEQ ID NO: 47 - protein, 9 aa, Synthetic construct
AYGEDDKII

SEQ ID NO: 48 - protein, 13 aa, Synthetic construct
AMRDLRDNFNKFY

SEQ ID NO: 49 - protein, 9 aa, Synthetic construct
AGGTGNQFY

SEQ ID NO: 50 - protein, 11 aa, Synthetic construct
AVTTDSWGKLQ

SEQ ID NO: 51 - protein, 12 aa, Synthetic construct
ALSGFYNTDKLI

SEQ ID NO: 52 - protein, 10 aa, Synthetic construct
AVNFGGGKLI

SEQ ID NO: 53 - protein, 11 aa, Synthetic construct
AMRGDSSYKLI

SEQ ID NO: 54 - protein, 9 aa, Synthetic construct
AFITGNQFY

SEQ ID NO: 55 - protein, 9 aa, Synthetic construct
ILDNNNDMR

SEQ ID NO: 56 - protein, 14 aa, Synthetic construct
AVGAPSGAGSYQLT

SEQ ID NO: 57 - protein, 11 aa, Synthetic construct
WGTYNQGGKLI

SEQ ID NO: 58 - protein, 11 aa, Synthetic construct
AMRGDSSYKLI

SEQ ID NO: 59 - protein, 11 aa, Synthetic construct
AVKSGGSYIPT

SEQ ID NO: 60 - protein, 11 aa, Synthetic construct
AVRTNSGYALN

SEQ ID NO: 61 - protein, 10 aa, Synthetic construct
AVNFGGGKLI

SEQ ID NO: 62 - protein, 11 aa, Synthetic construct
ATDDDSARQLT

SEQ ID NO: 63 - protein, 13 aa, Synthetic construct
AGQPLGGSNYKLT

SEQ ID NO: 64 - protein, 12 aa, Synthetic construct
IVVRSSNTGKLI

SEQ ID NO: 65 - protein, 11 aa, Synthetic construct
ASSLSFGTEAF

SEQ ID NO: 66 - protein, 11 aa, Synthetic construct
ASSDWVSYEQY

SEQ ID NO: 67 - protein, 14 aa, Synthetic construct
ASSLWEKLAKNIQY

SEQ ID NO: 68 - protein, 12 aa, Synthetic construct
ASSQRQEGDTQY

SEQ ID NO: 69 - protein, 13 aa, Synthetic construct
ASSGGHTGSNEQF

SEQ ID NO: 70 - protein, 13 aa, Synthetic construct
ASRYRDDSYNEQF

SEQ ID NO: 71 - protein, 11 aa, Synthetic construct
ASSLGQAYEQY

SEQ ID NO: 72 - protein, 13 aa, Synthetic construct
AWSETGLGTGELF

SEQ ID NO: 73 - protein, 12 aa, Synthetic construct
ATGTGDSNQPQH

SEQ ID NO: 74 - protein, 11 aa, Synthetic construct
ASSFNMATGQY

SEQ ID NO: 75 - protein, 12 aa, Synthetic construct
ASSMSAMGTEAF

SEQ ID NO: 76 - protein, 16 aa, Synthetic construct
ASSFTWTSGGATDTQY

SEQ ID NO: 77 - protein, 11 aa, Synthetic construct
ASSLGQAYEQY

SEQ ID NO: 78 - protein, 11 aa, Synthetic construct
ASGQGNFDIQY

SEQ ID NO: 79 - protein, 12 aa, Synthetic construct
ASSLLGGWSEAF

SEQ ID NO: 80 - protein, 11 aa, Synthetic construct
ASSIFGQREQY

SEQ ID NO: 81 - protein, 11 aa, Synthetic construct
ASSSRSSYEQY

SEQ ID NO: 82 - protein, 10 aa, Synthetic construct
ASSIGVYGYT

SEQ ID NO: 83 - protein, 10 aa, Synthetic construct
ASSIGVYGYT

SEQ ID NO: 84 - protein, 11 aa, Synthetic construct
SARDGTGNGYT

SEQ ID NO: 85 - protein, 13 aa, Synthetic construct
ASSLGQGLLYGYT

SEQ ID NO: 86 - protein, 10 aa, Synthetic construct
ASSARSGELF

SEQ ID NO: 87 - protein, 10 aa, Synthetic construct
ASSARSGELF

SEQ ID NO: 88 - protein, 13 aa, Synthetic construct
ASSLRGRGDQPQH

SEQ ID NO: 89 - protein, 10 aa, Synthetic construct
ASSYQGTEAF

SEQ ID NO: 90 - protein, 11 aa, Synthetic construct
ASSFRGGKTQY

SEQ ID NO: 91 - protein, 10 aa, Synthetic construct
ASRRGPYEQY

SEQ ID NO: 92 - protein, 12 aa, Synthetic construct
ASREGLGGTEAF

SEQ ID NO: 93 - protein, 12 aa, Synthetic construct
AISEVGVGQPQH

SEQ ID NO: 94 - protein, 14 aa, Synthetic construct
ASRPGLAGGRPEQY

SEQ ID NO: 95 - protein, 12 aa, Synthetic construct
ASPGLAGEYEQY

SEQ ID NO: 96 - protein, 12 aa, Synthetic construct
ASSRTGSTYEQY

SEQ ID NO: 97 - protein, 12 aa, Synthetic construct
ASSQTQLWETQY

SEQ ID NO: 98 - protein, 14 aa, Synthetic construct
ASSLAPGTTNEKLF

SEQ ID NO: 99 - protein, 12 aa, Synthetic construct
ASSPTSGIYEQY

SEQ ID NO: 100 - protein, 10 aa, Synthetic construct
ASSGASHEQY

SEQ ID NO: 101 - protein, 11 aa, Synthetic construct
ASSSWDTGELF

SEQ ID NO: 102 - protein, 12 aa, Synthetic construct
ASSYLNRDSALD

SEQ ID NO: 103 - protein, 11 aa, Synthetic construct
ASSDTVSYEQY

SEQ ID NO: 104 - protein, 11 aa, Synthetic construct
ASSLSFGTEAF

SEQ ID NO: 105 - protein, 15 aa, Synthetic construct
ASSANPGDSSNEKLF

SEQ ID NO: 106 - protein, 10 aa, Synthetic construct
ASSQDRDTQY

SEQ ID NO: 107 - protein, 9 aa, Synthetic construct
AAGIGILTV

SEQ ID NO: 108 - protein, 9 aa, Synthetic construct
ALWGFFPVL

SEQ ID NO: 109 - protein, 10 aa, Synthetic construct
ALWGPDPAAA

SEQ ID NO: 110 - protein, 13 aa, Synthetic construct
APRGPHGGAASGL

SEQ ID NO: 111 - protein, 10 aa, Synthetic construct
EENLLDFVRF

SEQ ID NO: 112 - protein, 9 aa, Synthetic construct
EEYLQAFTY

SEQ ID NO: 113 - protein, 10 aa, Synthetic construct
ELAGIGILTV

SEQ ID NO: 114 - protein, 11 aa, Synthetic construct
EPLPQGQLTAY

SEQ ID NO: 115 - protein, 9 aa, Synthetic construct
ESDPIVAQY

SEQ ID NO: 116 - protein, 9 aa, Synthetic construct
EVDPIGHLY

SEQ ID NO: 117 - protein, 9 aa, Synthetic construct
FEDLRVLSF

SEQ ID NO: 118 - protein, 9 aa, Synthetic construct
FLRGRAYGL

SEQ ID NO: 119 - protein, 9 aa, Synthetic construct
GILGFVFTL

SEQ ID NO: 120 - protein, 9 aa, Synthetic construct
GLCTLVAML

SEQ ID NO: 121 - protein, 10 aa, Synthetic construct
GTSGSPIVNR

SEQ ID NO: 122 - protein, 11 aa, Synthetic construct
HPVGEADYFEY

SEQ ID NO: 123 - protein, 9 aa, Synthetic construct
HSKKKCDEL

SEQ ID NO: 124 - protein, 9 aa, Synthetic construct
ILAKFLHWL

SEQ ID NO: 125 - protein, 9 aa, Synthetic construct
IPLTEEAEL

SEQ ID NO: 126 - protein, 10 aa, Synthetic construct
KLVALGINAV

SEQ ID NO: 127 - protein, 10 aa, Synthetic construct
KRWIILGLNK

SEQ ID NO: 128 - protein, 9 aa, Synthetic construct
LAGIGILTV

SEQ ID NO: 129 - protein, 9 aa, Synthetic construct
LLFGYPVYV

SEQ ID NO: 130 - protein, 13 aa, Synthetic construct
LPEPLPQGQLTAY

SEQ ID NO: 131 - protein, 10 aa, Synthetic construct
MMWDRGLGMM

SEQ ID NO: 132 - protein, 10 aa, Synthetic construct
MVWGPDPLYV

SEQ ID NO: 133 - protein, 9 aa, Synthetic construct
NLVPMVATV

SEQ ID NO: 134 - protein, 10 aa, Synthetic construct
RFPLTFGWCF

SEQ ID NO: 135 - protein, 10 aa, Synthetic construct
RQFGPDFPTI

SEQ ID NO: 136 - protein, 10 aa, Synthetic construct
RQFGPDWIVA

SEQ ID NO: 137 - protein, 10 aa, Synthetic construct
RQWGPDPAAV

SEQ ID NO: 138 - protein, 10 aa, Synthetic construct
RYPLTFGWCF

SEQ ID NO: 139 - protein, 9 aa, Synthetic construct
SLLMWITQV

SEQ ID NO: 140 - protein, 9 aa, Synthetic construct
SLYNTVATL

SEQ ID NO: 141 - protein, 10 aa, Synthetic construct
SMLGIGIVPV

SEQ ID NO: 142 - protein, 8 aa, Synthetic construct
TAFTIPSI

SEQ ID NO: 143 - protein, 9 aa, Synthetic construct
VMAPRTLIL

SEQ ID NO: 144 - protein, 10 aa, Synthetic construct
IAFQGAQKLV

SEQ ID NO: 145 - protein, 16 aa, Synthetic construct
LVGDGGSFSGGYNKLI

SEQ ID NO: 146 - protein, 13 aa, Synthetic construct
ILRDGRGGADGLT

SEQ ID NO: 147 - protein, 12 aa, Synthetic construct
AVDRGSTLGRLY

SEQ ID NO: 148 - protein, 11 aa, Synthetic construct
AALIQGAQKLV

SEQ ID NO: 149 - protein, 13 aa, Synthetic construct
AVSESPFGNEKLT

SEQ ID NO: 150 - protein, 12 aa, Synthetic construct
AVGASGNTGKLI

SEQ ID NO: 151 - protein, 12 aa, Synthetic construct
ALSGGDSSYKLI

SEQ ID NO: 152 - protein, 11 aa, Synthetic construct
TVYGGATNKLI

SEQ ID NO: 153 - protein, 14 aa, Synthetic construct
ALSLYSGAGSYQLT

SEQ ID NO: 154 - protein, 10 aa, Synthetic construct
ILRDRSNQFY

SEQ ID NO: 155 - protein, 11 aa, Synthetic construct
ILRDSRAQKLV

SEQ ID NO: 156 - protein, 11 aa, Synthetic construct
AVGETGANNLF

SEQ ID NO: 157 - protein, 13 aa, Synthetic construct
AVALNNNAGNMLT

SEQ ID NO: 158 - protein, 10 aa, Synthetic construct
AFKAAGNKLT

SEQ ID NO: 159 - protein, 13 aa, Synthetic construct
ATDAGYNQGGKLI

SEQ ID NO: 160 - protein, 13 aa, Synthetic construct
AASDTGANTGKLT

SEQ ID NO: 161 - protein, 11 aa, Synthetic construct
ASPQGGSEKLV

SEQ ID NO: 162 - protein, 13 aa, Synthetic construct
ASSFRALAADTQY

SEQ ID NO: 163 - protein, 11 aa, Synthetic construct
SAGVGGQETQY

SEQ ID NO: 164 - protein, 14 aa, Synthetic construct
ASSVAVSAGTYEQY

SEQ ID NO: 165 - protein, 10 aa, synthetic construct
ASSQIRETQY

SEQ ID NO: 166 - protein, 9 aa, Synthetic construct
ASTYHGTGY

SEQ ID NO: 167 - protein, 12 aa, Synthetic construct
ASSSTGLPYGYT

SEQ ID NO: 168 - protein, 13 aa, Synthetic construct
ASSLRDGYTGELF

SEQ ID NO: 169 - protein, 13 aa, Synthetic construct
ASSLADRVNTEAF

SEQ ID NO: 170 - protein, 12 aa, Synthetic construct
SARGGSYNSPLH

SEQ ID NO: 171 - protein, 12 aa, Synthetic construct
ASSLAQGGETQY

SEQ ID NO: 172 - protein, 14 aa, Synthetic construct
ASSTTPGTGTETQY

SEQ ID NO: 173 - protein, 13 aa, Synthetic construct
ASSAGTSGEYEQY

SEQ ID NO: 174 - protein, 12 aa, Synthetic construct
ASSEARRYNEQF

SEQ ID NO: 175 - protein, 12 aa, Synthetic construct
ASSVAPGSDTQY

SEQ ID NO: 176 - protein, 13 aa, Synthetic construct
ASSNRGLGTDTQY

SEQ ID NO: 177 - protein, 13 aa, Synthetic construct
ASSRLAGGMDEQF

SEQ ID NO: 178 - protein, 9 aa, Synthetic construct
ASSAGNTIY

SEQ ID NO: 179 - protein, 11 aa, Synthetic construct
ASGDFWGDTLY

SEQ ID NO: 180 - protein, 14 aa, Synthetic construct
ASSSGGWGGGTEAF

SEQ ID NO: 181 - protein, 13 aa, Synthetic construct
APQPELPYPQPGS

SEQ ID NO: 182 - protein, 11 aa, Synthetic construct
QPFPQPELPYP

SEQ ID NO: 183 - protein, 13 aa, Synthetic construct
SGEGSFQPSQENP

SEQ ID NO: 184 - protein, 15 aa, Synthetic construct
FELIGILNAAKVPAD

SEQ ID NO: 185 - protein, 15 aa, Synthetic construct
GELIGTLNAAKVPAD

SEQ ID NO: 186 - protein, 13 aa, Synthetic construct
PKYVKQNTLKLAT

SEQ ID NO: 187 - protein, 13 aa, Synthetic construct
QHIRCNIPKRISA

SEQ ID NO: 188 - protein, 15 aa, Synthetic construct
VHFFKNIVTPRTPGG

SEQ ID NO: 189 - protein, 16 aa, Synthetic construct
FSWGAEGQRPGFGSGG

SEQ ID NO: 190 - protein, 12 aa, Synthetic construct
QAFWIDLFETIG

SEQ ID NO: 191 - protein, 18 aa, Synthetic construct
APSGEGSFQPSQENPQGS

SEQ ID NO: 192 - protein, 16 aa, Synthetic construct
APSGEGSFQPSQENPQ

SEQ ID NO: 193 - protein, 11 aa, Synthetic construct
QPQQSFPEQEA

SEQ ID NO: 194 - protein, 11 aa, Synthetic construct
GPQQSFPEQEA

SEQ ID NO: 195 - protein, 12 aa, Synthetic construct
RFYKTLRAEQAS

SEQ ID NO: 196 - protein, 19 aa, Synthetic construct
VEELYLVAGEEGCGGGGSL

SEQ ID NO: 197 - protein, 13 aa, Synthetic construct
RVSYYGPKTSPVQ

SEQ ID NO: 198 - protein, 12 aa, Synthetic construct
APFSEQEQPVLG

SEQ ID NO: 199 - protein, 11 aa, Synthetic construct
ILSGGSNYKLT

SEQ ID NO: 200 - protein, 11 aa, Synthetic construct
ILSGGCNYKLT

SEQ ID NO: 201 - protein, 9 aa, Synthetic construct
ASSFGREQY

SEQ ID NO: 202 - protein, 9 aa, Synthetic construct
ASSFCREQY

SEQ ID NO: 203 - protein, 10 aa, Synthetic construct
SSLENFRAYV

SEQ ID NO: 204 - protein, 10 aa, Synthetic construct
SSLCNFRAYV

SEQ ID NO: 205 - protein, 10 aa, Synthetic construct
SSKENFKAYV

SEQ ID NO: 206 - protein, 10 aa, Synthetic construct
SSLENFAAYV

SEQ ID NO: 207 - protein, 10 aa, Synthetic construct
SSLENFLAYV

SEQ ID NO: 208 - protein, 10 aa, Synthetic construct
SSLCNFKAYV

SEQ ID NO: 209 - protein, 10 aa, Synthetic construct
SSLCNFAAYV

SEQ ID NO: 210 - protein, 10 aa, Synthetic construct
SSLCNFLAYV

SEQ ID NO: 211 - protein, 15 aa, Mus musculus
KKDWVSLWKGFSFKK

SEQ ID NO: 212 - protein, 15 aa, Synthetic construct
KKDWVCLWKGFSFKK

SEQ ID NO: 213 - protein, 15 aa, Synthetic construct
KKDWVSLWKGFCFKK

SEQ ID NO: 214 - protein, 15 aa, Synthetic construct
KKDCVSLWKGFSFKK

SEQ ID NO: 215 - protein, 15 aa, Synthetic construct
KKDWVSLWCGFSFKK

SEQ ID NO: 216 - protein, 18 aa, Mus musculus
KKENPVVHFFKNIVTPKK

SEQ ID NO: 217 - DNA, 20 bp, Synthetic construct
  source 1..20: mol_type = other DNA
cgtctgaactggggtaggtg

SEQ ID NO: 218 - DNA, 20 bp, Synthetic construct
  source 1..20: mol_type = other DNA
ctgaaagcccatggaactgc

SEQ ID NO: 219 - DNA, 30 bp, Synthetic construct
  source 1..30: mol_type = other DNA
  modified_base 28..30: mod_base = OTHER; note = each N base is riboguanosine
gaattcacctatcaacgcagagtacatnnn

SEQ ID NO: 220 - DNA, 26 bp, Synthetic construct
  source 1..26: mol_type = other DNA
aattgaattcacctatcaacgcagag

SEQ ID NO: 221 - DNA, 30 bp, synthetic construct
  source 1..30: mol_type = other DNA
aattctcgagaagtcggtgaacaggcagag

SEQ ID NO: 222 - DNA, 30 bp, Synthetic construct
  source 1..30: mol_type = other DNA
aattctcgagtggacctccttgccattcac

SEQ ID NO: 223 - DNA, 20 bp, synthetic construct
  source 1..20: mol_type = other DNA
cctcacattgccaaaagacg

SEQ ID NO: 224 - protein, 10 aa, Synthetic construct
ALSSGSWQLI

SEQ ID NO: 225 - protein, 13 aa, Synthetic construct
ASGEGQGRGERLF

SEQ ID NO: 226 - protein, 15 aa, Synthetic construct
KKDWVSCWKGFSFKK